(19) United States
(12) Patent Application Publication
(10) Pub. No.: US 2022/0186244 A1
(43) Pub. Date: Jun. 16, 2022

(54) ROSE ROSETTE VIRUS INFECTIOUS CLONES AND USES THEREOF

(71) Applicant: The Texas A&M University System, College Station, TX (US)

(72) Inventors: Jeanmarie Verchot, Plano, TX (US); David Mingxiong Pang, Plano, TX (US)

(21) Appl. No.: 17/437,242

(22) PCT Filed: Mar. 8, 2020
(86) PCT No.: PCT/US2020/021611

§ 371 Date: Sep. 8, 2021

Related U.S. Application Data
(60) Provisional application No. 62/815,734, filed on Mar. 8, 2019.

(51) Int. Cl. C12N 15/82; C12N 7/00; C12N 15/113

(52) U.S. Cl. CPC C12N 15/8271; C12N 7/00; C12N 2760/12043; C12N 15/8205; C12N 2760/12021; C12N 15/1131

(57) ABSTRACT

Disclosed herein is the first infectious clone of a member of the Emaravirus genus of multipartite negative strand RNA virus. In particular, disclosed herein is an infectious clone of Rose rosette virus (RRV). This method can in some embodiments be used to prepare infectious clones of any species within the Fimoviridae family, such as any species within the Emaravirus genus.

### CROSS-REFERENCE TO RELATED APPLICATIONS

This application claims benefit of U.S. Provisional Application No. 62/815,734, filed Mar. 8, 2019, which is hereby incorporated herein by reference in its entirety.

### SEQUENCE LISTING

This application contains a sequence listing filed in electronic form as an ASCII.txt file entitled “922001-1080 Sequence Listing_ST25” created on Feb. 25, 2022, and having 91,536 bytes. The content of the sequence listing is incorporated herein in its entirety.

### BACKGROUND

Infectious clone technology has been slow to develop for viruses with negative strand RNA genomes because the naked genomic or antigenomic RNAs are not able to initiate infection by themselves. The minimum infectious unit for this type of virus requires a ribonucleoprotein (RNP) complex composed of viral genomic RNA and RNA dependent RNA polymerase (P proteins). Among negative strand RNA viruses, the first infectious clones were produced for viruses with non-segmented genomes belonging to the families Rhabdoviridae, Paramyxoviridae, and Filoviridae (Ebola). However, no infectious clones of a multipartite negative strand RNA virus have been reported.

### SUMMARY

Disclosed herein is the first infectious clone of a member of the Emaravirus genus of multipartite negative strand RNA virus. In particular, disclosed herein is an infectious clone of Rose rosette virus (RRV). This method can in some embodiments be used to prepare infectious clones of any species within the Fimoviridae family, such as any species within the Emaravirus genus.

Disclosed herein is a DNA polynucleotide encoding a Fimoviridae virus antigenomic RNA (agRNA) that is complementary to an RNA genome segment of the Fimoviridae virus for used in the disclosed infectious clones.

In some embodiments, the Fimoviridae virus is an Emaravirus virus selected from the group consisting of a Rose Rosette Virus (RRV), Actinidia chlorotic ringspot-associated virus (AcCRaV), European mountain ash ringspot-associated virus (EMARaV), fig mosaic virus (FMV), High Plains wheat mosaic virus (HPWMoV), pigeonpea sterility mosaic virus (PPSMV), pea sterility mosaic virus 2 (PPSMV-2), raspberry leaf blotch virus (RLBV), redbud yellow ringspot-associated virus (RYRaV).

In some embodiments, the RNA genome segment is an RNA1, RNA2, agRNA3, RNA4, RNA5, RNA6, RNA7, or any combination thereof. Therefore, in some embodiments, the agRNA is an agRNA1, agRNA2, agRNA3, agRNA4, agRNA5, agRNA6, agRNA7, or any combination thereof.

In particular embodiments, the Fimoviridae virus is a Rose Rosette Virus (RRV). Therefore, in some embodiments, the agRNA is 70-100% identical to a polynucleotide that is complementary to any one of SEQ ID NOs: 4, 6, 8, 10, 12, 15, or 17.

In some embodiments, the agRNA is operatively linked to a transcription control sequence and a self-cleaving ribozyme, wherein the agDNA is configured to produce viral transcripts with authentic 5′ and 3′ ends. Promoters can be near-constitutive, tissue-specific, developmentally specific promoters. Suitable promoters may be obtained from plants, plant viruses, or plant commensal, saprophytic, symbiotic, or pathogenic microbes and include, but are not limited to, the nopaline synthase (NOS) and octopine synthase (OCS) promoters, the cauliflower mosaic virus (CaMV) 19S and 35S promoters, the light-inducible promoter from the small subunit of ribulose 1,5-bisphosphate carboxylase, the rice Act1 promoter, the Figwort Mosaic Virus (FMV) 35S promoter, the sugar cane bacilliform DNA virus promoter, the ubiquitin (UBI) promoter, the peanut chlorotic streak virus promoter, the comalina yellow virus promoter, the chlorophyll a/b binding protein promoter, and meristem enhanced promoters Act2, Act8, Act11 and EF1a and the like. All of these promoters have been used to create various types of DNA constructs which have been expressed in plants.

Non-limiting examples of self-cleaving ribozymes include hammerhead, hairpin, hepatitis delta virus (HDV), Varkud satellite (VS), and glmS ribozymes. For example, in embodiments, the self-cleaving ribozyme is HDV ribozyme.

In some embodiments, the disclosed DNA polynucleotides are incorporated in a plasmid that contains T7, SP6, RNA pol I, and RNA pol II promoters. For example, the plasmid can be a pCB301 plasmid.

Also disclosed herein are agrobacterium cells transformed with DNA polynucleotides disclosed herein. For example, an agrobacterium can be produced for each agRNA to be used for infection.

Also disclosed herein is an infectious Fimoviridae virus composition comprising a plurality of Agrobacterium transformed with DNA polynucleotides disclosed herein. As disclosed herein, infection requires at least agRNA1, agRNA2, agRNA3, and agRNA4. Therefore, the infectious Fimoviridae virus composition can contain at least a first Agrobacterium transformed with a DNA polynucleotide encoding agRNA1, a second Agrobacterium transformed with a DNA polynucleotide encoding agRNA2, a third Agrobacterium transformed with a DNA polynucleotide encoding agRNA3, and a fourth Agrobacterium transformed with a DNA polynucleotide encoding agRNA4. In some embodiments, infectious Fimoviridae virus composition also contains a fifth Agrobacterium transformed with a DNA polynucleotide encoding agRNA5, a sixth Agrobacterium transformed with a DNA polynucleotide encoding agRNA6, a seventh Agrobacterium transformed with a DNA polynucleotide encoding agRNA7, or any combination thereof.

In some embodiments, agRNA5, agRNA6, agRNA7, or any combination thereof, is used to deliver a transgene or a non-coding RNA. In some embodiments, this can be done for gene silencing and/or gene editing. In some embodiments, this can be done to increase plant growth, increase fruit or seed yield increase stress tolerance, or provide some other benefit to plant health or performance.

Therefore, in some embodiments, the ORF of agRNA5, agRNA6, agRNA7, or any combination thereof, has been replaced with a transgene or non-coding RNA operably linked to an agRNA56, agRNA6, or agRNA7 viral promoter. In some embodiments, the transgene encodes a regulatory gene involved in transactivation of stress-responsive genes, stomatal movement, plant stress physiology, or a combination thereof. In some embodiments, the transgene provides drought tolerance, cellular protection/detoxification, transpiration control, or a combination thereof.

One surprising effect was the ability of the infectious Fimoviridae virus composition to be deliverable by spray, such as a airbrush. In some embodiments, the agrobacterium cells are suspended in an infiltration solution, which can then be sprayed onto the surface of a plant to be infected. In some embodiments, the infiltration solution comprises a surfactant, such as Silwet-77 (polyalkyleneoxide modified heptamethyltrisiloxane (84%) and allyloxypolyethyleneglycol methyl ether (16%)) or Pluronic F-68.

Also disclosed herein is a method for inoculating a plant that involves administering to the plant the infectious Fimoviridae virus composition disclosed herein. In some embodiments, the method does not require co-administering to the plant a source of viral replicase, nucleocapsid (NC) proteins, or silencing suppressor proteins. Another unexpected finding was the ability of the disclosed polynucleotides to produce infectious clones without a vector providing these proteins in trans. Likewise, in some embodiments, the method does not require the use of a mite vector. Likewise, in some embodiments, the method does not require grafting.

The disclosed infectious Fimoviridae virus can be used to infect any plant type, including species from the genera Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Zea, Avena, Hordeum, Secale, Triticum, Sorghum, Picea, Sorbus aucuparia, Vitis vinifera, and Populus.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-10 show a photographic image that can demonstrate the use of a hand-held artist airbrush to deliver sap inoculum to rose plants (FIG. 1A), an image of a gel that can demonstrate RT-PCR results that can verify the presence of antigenomic RNA1, RNA3, RNA4, RNA5, RNA6, and RNA7 in inoculated Arabidopsis and N. benthamiana. Virus infected rose plants were used as a positive control in these experiments (FIG. 1B), and microscopic images that can demonstrate the results of dsRBFC assay in mock treated and RRV infected N. benthamiana leaves (FIG. 10). dsRBFC was carried out for fluorescence labelling RRV dsRNA replication intermediates. Scale bar is 100 μm.

FIG. 2 shows a diagrammatic representation of antigenomic RRV constructs. The lines represent the 3′ to 5′ orientation of the genome segments. The open boxes indicate the open reading frames encoded by each segment. The size in base pairs for each segment is provided. The modifications are where GFP or iLOV were inserted into the genome are also identified.

FIGS. 3A-3G shows various results from infecting plants with the RRV infectious clones described herein. FIG. 3A shows the morphology of plants that are healthy (on left) or infected with RRV infectious clone at 35 days post inoculation. FIG. 3B shows healthy plants produce 3 inflorescences, and FIG. 3C produce more than 3. FIG. 3D shows the PCR gels confirm the plants are infected using primers that amplify RNA 4 sequences. Actin was used as an internal PCR control. FIGS. 3E-G, The arrows in images highlight aerial rosette leaves that occur in infected plants. This does not occur in healthy plants.

FIGS. 4A-4H shows healthy and virus infected plants at 12 and 35 days Infected N. benthamiana plants do show necrosis, but also more flowers than the healthy control. FIGS. 4 D-H shows florescent micrographs showing GFP in infected leaves.

FIGS. 5A to 5J show experimental results of infectious clones in garden rose.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to cy′”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used in the specification and the appended claims, the singular forms “a,” “an,” and “the” include plural referents unless the context clearly dictates otherwise.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +/−10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Embodiments of the present disclosure will employ, unless otherwise indicated, techniques of molecular biology, microbiology, virology, plant physiology, biochemistry, genetic engineering and the like, which are within the skill of the art. Such techniques are explained fully in the literature.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible unless the context clearly dictates otherwise.

### Definitions

As used herein, “antigenomic RNA” refers to the complementary strand of RNA from which the genome of a virus is constructed. Thus, in a negative strand virus, the antigenomic RNA is the positive RNA strand and in a positive RNA strand virus, the antigenomic RNA is the negative RNA strand.

As used herein, “cDNA” refers to a DNA sequence that is complementary to an RNA transcript in a cell. It is a man-made molecule. Typically, cDNA is made in vitro by an enzyme called reverse-transcriptase using RNA transcripts as templates.

As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “deoxyribonucleic acid (DNA)” and “ribonucleic acid (RNA)” can generally refer to any polyribonucleotide or polydeoxribonucleotide, which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA) or coding mRNA (messenger RNA).

As used herein, “DNA molecule” can include nucleic acids/polynucleotides that are made of DNA.

As used herein, the term “encode” refers to principle that DNA can be transcribed into RNA, which can then be translated into amino acid sequences that can form proteins.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also be a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

As used herein, “identity,” can refer to a relationship between two or more nucleotide or polypeptide sequences, as determined by comparing the sequences. In the art, “identity” can also refer to the degree of sequence relatedness between nucleotide or polypeptide sequences as determined by the match between strings of such sequences. “Identity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48: 443-453,) algorithm (e.g., NBLAST, and XBLAST). The default parameters are used to determine the identity for the polypeptides of the present disclosure, unless stated otherwise.

As used herein, “negative strand RNA virus” refers to a virus that has a single stranded of RNA as its genome and has to be transcribed as soon as the virus enters the host in order to carry out viral replication.

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, “operatively linked” in the context of recombinant DNA molecules, vectors, and the like refers to the regulatory and other sequences useful for expression, stabilization, replication, and the like of the coding and transcribed non-coding sequences of a nucleic acid that are placed in the nucleic acid molecule in the appropriate positions relative to the coding sequence so as to effect expression or other characteristic of the coding sequence or transcribed non-coding sequence. This same term can be applied to the arrangement of coding sequences, non-coding and/or transcription control elements (e.g. promoters, enhancers, and termination elements), and/or selectable markers in an expression vector. “Operatively linked” can also refer to an indirect attachment (i.e. not a direct fusion) of two or more polynucleotide sequences or polypeptides to each other via a linking molecule (also referred to herein as a linker).

As used herein, “organism”, “host”, and “subject” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). These terms also contemplate plants, fungi, bacteria, etc.

As used herein, “overexpressed” or “overexpression” refers to an increased expression level of an RNA and/or protein product encoded by a gene as compared to the level of expression of the RNA or protein product in a normal or control cell. The amount of increased expression as compared to a normal or control cell can be about 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, 2.0, 2.2, 2.4, 2.6, 2.8, 3.0, 3.3, 3.6, 3.9, 4.0, 4.4, 4.8, 5.0, 5.5, 6, 6.5, 7, 7.5, 8.0, 8.5, 9, 9.5, 10, 11, 12, 13, 14, 15, 16, 17, 18, 19, 20, 25, 30, 35, 40, 45, 50, 60, 70, 0, 90, 100 fold or more greater than the normal or control cell.

As used herein, the terms “optional” or “optionally” means that the subsequently described event or circumstance can or cannot occur, and that the description includes instances where said event or circumstance occurs and instances where it does not.

As used herein, “plasmid” refers to a non-chromosomal double-stranded DNA sequence including an intact “replicon” such that the plasmid is replicated in a host cell.

As used herein, “polypeptides” or “proteins” refers to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, VV), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be required for the structure, function, and regulation of the body's cells, tissues, and organs.

As used herein, “positive strand RNA virus” refers to viruses with single stranded genomes that are such polarity that they can be directly translated in a host cell.

As used herein, “promoter” includes all sequences capable of driving transcription of a coding or a non-coding sequence. In particular, the term “promoter” as used herein refers to a DNA sequence generally described as the 5′ regulator region of a gene, located proximal to the start codon. The transcription of an adjacent coding sequence(s) is initiated at the promoter region. The term “promoter” also includes fragments of a promoter that are functional in initiating transcription of the gene.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein, “selectable marker” refers to a gene whose expression allows one to identify cells that have been transformed or transfected with a vector containing the marker gene. For instance, a recombinant nucleic acid may include a selectable marker operatively linked to a gene of interest and a promoter, such that expression of the selectable marker indicates the successful transformation of the cell with the gene of interest.

A “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed.

As used herein, “transforming” when used in the context of engineering or modifying a cell, refers to the introduction by any suitable technique and/or the transient or stable incorporation and/or expression of an exogenous gene in a cell. It can be used interchangeably in some contexts herein with “transfection”.

As used herein, the term “transfection” refers to the introduction of an exogenous and/or recombinant nucleic acid sequence into the interior of a membrane enclosed space of a living cell, including introduction of the nucleic acid sequence into the cytosol of a cell as well as the interior space of a mitochondria, nucleus, or chloroplast. The nucleic acid may be in the form of naked DNA or RNA, it may be associated with various proteins or regulatory elements (e.g., a promoter and/or signal element), or the nucleic acid may be incorporated into a vector or a chromosome.

As used herein, “variant” can refer to a polynucleotide or polypeptide that differs from a reference polynucleotide or polypeptide, and retains essential and/or characteristic properties (structural and/or functional) of the reference polynucleotide or polypeptide. A typical variant of a polypeptide differs in amino acid sequence from another, reference polypeptide. The differences can be limited so that the sequences of the reference polypeptide and the variant are closely similar overall and, in many regions, identical. A variant and reference polypeptide may differ in nucleic or amino acid sequence by one or more modifications at the sequence level or post-transcriptional or post-translational modifications (e.g., substitutions, additions, deletions, methylation, glycosylation, etc.). A substituted nucleic acid may or may not be an unmodified nucleic acid of adenine, thiamine, guanine, cytosine, uracil, including any chemically, enzymatically or metabolically modified forms of these or other nucleotides. A substituted amino acid residue may or may not be one encoded by the genetic code. A variant of a polypeptide may be naturally occurring such as an allelic variant, or it may be a variant that is not known to occur naturally. “Variant” includes functional and structural variants.

As used herein, the term “vector” is used in reference to a vehicle used to introduce an exogenous nucleic acid sequence into a cell. A vector may include a DNA molecule, linear or circular (e.g. plasmids), which includes a segment encoding a polypeptide of interest operatively linked to additional segments that provide for its transcription and translation upon introduction into a host cell or host cell organelles. Such additional segments may include promoter and terminator sequences, and may also include one or more origins of replication, one or more selectable markers, an enhancer, a polyadenylation signal, etc. Expression vectors are generally derived from yeast or bacterial genomic or plasmid DNA, or viral DNA, or may contain elements of both.

As used herein, “wild-type” is the typical form of an organism, variety, strain, gene, protein, or characteristic as it occurs in nature, as distinguished from mutant forms that may result from selective breeding or transformation with a transgene.

As used herein, “electroporation” is a transformation method in which a high concentration of plasmid DNA (containing exogenous DNA) is added to a suspension of host cell protoplasts, and the mixture shocked with an electrical field of about 200 to 600 V/cm.

As used herein, a “transgene” refers to an artificial gene which is used to transform a cell of an organism, such as a bacterium or a plant.

As used herein, the term “exogenous DNA” or “exogenous nucleic acid sequence” or “exogenous polynucleotide” refers to a nucleic acid sequence that was introduced into a cell, organism, or organelle via transfection. Exogenous nucleic acids originate from an external source, for instance, the exogenous nucleic acid may be from another cell or organism and/or it may be synthetic and/or recombinant. While an exogenous nucleic acid sometimes originates from a different organism or species, it may also originate from the same species (e.g., an extra copy or recombinant form of a nucleic acid that is introduced into a cell or organism in addition to or as a replacement for the naturally occurring nucleic acid). Typically, the introduced exogenous sequence is a recombinant sequence.

Discussion

Roses are the economically most important ornamental plants belonging to the family Rosaceae and comprise 30% of the floriculture industry. Rose rosette virus has been devastating roses and the rose industry in the USA, causing millions of dollars in losses. Typical symptoms of RRV are described as rapid stem elongation, followed by breaking of axillary buds, leaflet deformation and wrinkling, bright red pigmentation, phyllody, and increased thorniness. As such, there exists a need for compositions and techniques for prevention and treatment of RRV in roses.

Described herein are infectious clones of RRV that can include one or more reporter genes that can act as an enhanced visual reporter system, which can useful for screening rose germplasm stocks, intermediate vectors, and other infected plants to identify new sources of resistance and monitor and control infection. The RRV infectious clones can also be used as a gene delivery platform for transient and stable transformation of plants. The RRV infections clones can also be applied to non-rose plants and can cause an improvement in one or more performance characteristics (e.g. growth or yield). Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Infectious RRV Recombinant Polynucleotides and Vectors

Described herein are recombinant polynucleotides that can encode one or more antigenomic (ag) RNA segments of the RRV and vectors that can contain one or more of the recombinant polynucleotides that can encode one or more agRNA segments of the RRV. RRV is a negative strand virus that is composed of 7 RNA segments (denoted herein as RNA1, RNA2, RNA3, RNA4, RNA5, RNA6, and RNA7). It will be appreciated that the agRNA sequences of RNA1-RNA7 (denoted herein as agRNA1, agRNA2, agRNA3, agRNA4, agRNA5, agRNA6, and agRNA7) are the complementary sequences to RNA1-RNA7. One of skill in the art will instantly appreciate the complementary sequences in view of the sequences provided and described herein.

In some embodiments, the RNA1 has the nucleic acid sequence:

(SEQ ID NO: 4)
AGTAGTGTTCTCCCTTAAATCATTCTAATCTAGACAAAATCCAAAAGAA
AGCAATAAAGGTCTAAAAGAAATAGTGCGTGTAATTTATCTAAAATTCT
AGTTCATCGTTCATATCTATAAAATCATGTATATAAAATTTAAAAATCA
AGATTATGAATAGTATAATCTTCTCAATTGGGTCACTTTGATGTCTATA
TTTCATAATTGTAATGAACAACCTGTGATATGGTGCAATCTCATTAGCA
CGATAATTCTTTATCACTTTGTCAGCACATGTTTCACCTAATTTGATTT
TGTTACAACTATTGATGAATCTATCTGGATCTTTAGATGCCATGACATC
TATATTTTGACCTAATGCTATCAATTGGACACACTCACACAGATTGCCC
ATATAAGAATTACCATGCTCATGGAAATAATTGCTAAAAAAGATCAAAT
TGCCAAACTTCTGTGGGTTGATTCTTTCAGTGACAATATCAAATTCAGT
GTAACCAGTCATTAATGATATTAGCTGGTTAGGTCCAAGATCTATTAGT
ACAGGCTTCAAGTAATCAGGGTATTTATGGAAAAGATTAGTTTCACATA
GATAACCCAATATATAATTACGTTCTATGTGTAAATTGTCTATTAAACA
GTTTAAGTAATACTCATCATCTATATAAGTCCTGAGTCTTACATCAAAT
GCCAACTCTTCATGATTGGTTGTTATAGTTAAAATCAGTTTAACATTTC
CATCCACTTCTATATAACTAGGTTGAACTTCTATGATTTTAGCATAGTG
TATTGGGCATACATTAAAGACTCGATCTCCAAAGTGGTTTGTATATCTT
CCATATTCATTTATATTAAACACATGATACCTCTCAGTGGGTGTGATAT
TACTTGTGATTTTAGAGATTCTCAGCAACTCTACATAATCTGATGCTAT
TTTCTTTTTTATTGATTCTAATGCACCGTAATCAGTTCGTAAATATTTG
TAATACATCATTGAAAATACAACCACATTGTTTTGAGTAAATGTTCTAA
CCTTCATTACATTACCTTGACACATGTAATATGCTAGTGATGCATTTTT
ATCATTTGCTTCAGAGTTTGTTGGTATTAACCAATGGTTGTATACTCTA
TGGTCAGTTATTAGCGAAGTTATGAATCTTGGGAATTCTATTTTACCCA
TCATGAAGAGTAATGCACTACTATTAATATCTTTCCCATTTTGCAACAC
ATTTAACAGCTCATGGGAATTGTAGTGACCACTCATAAATTCAGGGTGT
TCTTTTAATCTGTTGTAGAGGTTCAGATTTCTTGCTATTAATTTTGTTA
TGAATGCTAATGGTTCGTATGTATCTCTATAGGTTTGTAGGTTGCTTGC
TATTTTACCACTAGATGCAATCTTTATTTTATAACCCAACTTGTATTTA
ATATGAAAGTCATTATATGCATACATTCCATAACGTGTAAGAAGGTACT
CATCATACTTAGTTGACTTACTGTTTGATAAATACACCTTGGTGCTTAT
CTCATCTTTCTTTAGTAAAGAATCTATTGTCATAATTAAAGATGAGGGT
GATGGATAATTAGGTATGTTGAAACTTGGGTTGTTTAAAGTTCTTTTAA
TATCATTATAATATCGCACTAATAAAGACATAAACTTTAGTTTATTCTT
GTATGCCATAAAGCCAACTTCTCCAAGATCTGTGATAGGTTCAACGTCA
ATGGCAATGTCAGGTGAATTAGGATATTTGAATTTATAATATGCAATAT
AATCATCATCTATATCCTCTTGATTTTCATATATTTTTGTATCACCTAT
TCTTTTTAAAACATAATCACATATGTCAATGAATTTATCCGACTTAGGA
TCAATCGAGTTAATTATACAATACCTAGAAGTTAATAAAGATTGTATCA
ATGATGTATTAGAATATTTACCAAAGTCTTCAGCATAAATGCTCCTTGG
TTGTATAACTTTAGTGTATGATGGTCTTGCCATTGTGACAGTTTCTACT
TTAGAATAGAAAACCTGCATAGCCATTAGTATTTTTTTATCACTTAAAA
GGTATAAACTTAAGTACCTCTGTAAATCGTTAGCAGTTAGTGTCACTTC
ATCTATCTTAGAAGATAAATCTTTATATACTGTACCTATGGTTTTAGCC
TTGTCCTTTTCCATAACATGGGAACTTATAGTATACATGTTTTTATTTG
AACCAATAATTCTTCTGCCATAATCAATTGCAGGAGTTGAAAAAATTAA
TCCATCTCTAAAATTAGGGTTAGTATAATCCTGCAAGATGTTTGCTTTG
ATCAAATCTGGATCTGTAGGCTTCTTCACACACCATTCAGGATGAATAG
CTGCTTTAAGCCGTATCTCACTTTCATGCTCAAGATATTTCTTGTATGT
GTATGTCTTCTTTAATCTTAGACCCTTGTTTAAGCTGACAACATTGATA
ATGGACTTTTGACTCAAGTCATAGTCAATTATGTTATATGGATCAGCAT
CATCTCTTTCATATTGTGTATAATCCATACACAGGATACAAGATTTTAT
ATATTTATACACTTTGGGGCTATTAACTCTGGTTATTTCAAGATATTTC
TCAATTGTGTTAACATCTAACATATCTTCAATTAACTCTGTCTGTAGGA
TCTTAAACTTATTTAGTTTTTCTATTATATCACTTAAAATATTAAATGC
ATCTGCTGCATAATATGGAATCATACCTGCTAAATTTAATGGCAACTTG
TATCTTGGATATATTTGTATTGGTAAATCAGAGCTATCAATGTGAAACC
TTGGGTTTTTATCTGATGTGTATTGCATGTTGTAAGTTGACACAGTCAA
ATGGTTTATTAAAATAACTGAAGTTTTGATCATATTTAAAGGACATGCA
TGGGAAAATGCATTGTTGATATATCCTGAATATGATGCTAAATCTTGCA
TAGGTGAAGCATAACTTGTATCAGATGTCAAAGGTAACAGATCTGCCAA
ATAAAAGAAAAAAAGTTCATTTCCTACAATTATAGTAGACAAAAATTCT
TTATAAAATGTACTTATATAAGTTTTTTTCTCATTTAATGTGATGCAAT
GGAATTTATTAGAAAATGTTATTAAAGCAATAATCAATTTTCCAATATT
AGTTTTGTTTATTATACCTTTGTTTATGGCTTTTTGTTGATTTTTTGTC
CCTGTACAGATTAAGAAATCATATGTGGAATCATCAGAATGAACCATAG
ATGTCATGTTACACTCTAGCCTATTGTGATCTGAAAATATCTTGAGCAT
TGCTTCTGTATATAGAGTAGAACAATGATGAACAAAAGAAGAAATCATG
TTTAGATTACCTTGTAGCCAATTACTTCTAACTGTGAAATAATTTTGTT
TAAAATCATTTGTCATTTCTTCATATGATGTGTTTGCACTGTTGACATT
AACTAAATTGAGCATATCATTGAAGATGTTATCTGTGAGGACTATATTT
CTTTTATAATATCGAAAACATAAAAATGTTAAGAACCACTTTTCATCAG
GATGTAAAAATGGGTTGGTAGAGATCACAATGATAAATTTTAGGAATAA
ATCTCGAGCTGACCATTTTGATGCATCTGATGAAACTGAATATATTTCA
GCATCTTTATTATCCCTAATCTTCTGTCTTTTTTCTTTTATCAAGGTTA
ACCGTTGTTCTAATAGTTTTTTTTGTTTTTGATCCCCACTTATAGTTAT
AGCTTCACCAGGTATGTGTTTACATATAGCTTTGTATGTTTTCTCTATT
GGATAAAGACACAATCTAGTTTGTGCATTACCAGTATAGATTTCCCGAT
CATCAGCTGTTCTTTGGTCTTTATAAAATATTCTTATAAGTAGGTCATC
ATCATGGATCATCTTTTTATAGGCATCTTTTAAGCTCAATAAACCATAC
TCCTCAGTTAACTTGTAAAACTCATCAAAGACTTTAGCATTACTCTGTT
TTATGTAACCACCATTTACCATTCTTGTATATTGATGTCTTTTTATCTT
TATAAATCTAACACCCTTTATTGTTTCTATGTATATCTCAGGAAGAAGT
GTAACATTCATATCAGGGTTCTTTTGCTCCTCCTTTAATCGTTTAGCAT
TAATCCTATAAACTTCAGAATTAATTAGTTGCATGAATTGGTGATCATC
TTCAATTATAGATTCATTAATAATCTTCTCTAGTATCTGTATATCTGTA
CTATCTTTTAAACGACTAGGTGCTGGCACATAATCAGTTTTGGTGTTTG
ATACCATAGATTTTGTGCTAGAAAACTGCTTAAGTGATAATACTGGTTT
ATCAAATTCTAATTCACTACATATGCTCTGTCGAACTGCATCCTTTTTG
TTAAATAAATTGGCATATGCAACCATAGATGTTTTTTTCATCACATTGT
ATGAAATGGACATAGATGAATTATTACCAGTTTCTTGTAATATTGTGCC
ATATTTATCTAACATTTCACTATATTCCTTTTCAAATTTATATGGTGTA
TGATATAAGTTCAATAATTCTTGTGGAGAGCCATGTAAGCCTTTATTAC
CTAGATAAAAAAGCATGAATGCTTCATGGATTATTTCTTTGGGATTATC
AACACTGAGATTAGATATTGGTAGCTTTAGACTCAAGTTGTTGTTGAAA
CCTGTATTTTGTAATTCACCCATATCATCTATTTCTTCATTTCTTTTTT
TTTGATTGATCACTTTGAGCTGTTGTGTTGCCTTAACAATACCATCTAG
ACACCTTTTTATTATGTATGCATGTCCTAGTGTTGTTGGTCTAGATTCT
AATTTATCATCTATAAGATTATCTATATTAGAATAATCCGAATATACAG
CCTTAATGAAATTCTTATATGTATCTGTAACAGTTAATGATGCTATTGT
TATAAACTGGCATAGTATCCATATCATAAAGTGTATATCATGTTTGATC
TTTTTTTTCATGTTGGAATAATAAGTCATAATTAGGCAATACTTTCCGA
AAGAATGGTTAAGTAGTTTAAGTCTAGTCACATCCAAACTGATTACTTT
TGATAGAATGATGTTGTAGTTTCTTCCATGTATCAACTCATGTGCTACA
CCTAATAGGCGATTTGCTTCAAAACTTACAAGGTCTTTTTTAGATATAA
TAGTTAGAGTAAAGTACCTTAAAGGGGCTGATTTAAGTGTGTCAGAGTT
TGGCAGCGTTATGAGTATAGTGCATGGATCTGCTGTTTGTACCAACCTA
TACTTGTGTGAATTGACAGTATTTAACGATATCAAAGCTTTAAAGATGT
TATGTTGACTATATAAGTGATCTAAATATTTAGTTCTGCAAACTTCTGG
CAAATCACATATGTGTGTATTCATATTGTCTGAGTCTATAGAGACTAGT
TCATTGGCATAAGTTCCGACATGGTATTCACTATTGAATATTTCACCCA
TGTATCTTTTTAGAGTTATTATATCCTCTTCACTGTCAGCAACTGATAT
ACAATCTTTCTTCAACACCTTTTCATTGTAGTGCTTATTTTTGAAACCT
ATAATATTATCTTTCATATGTTTGTCCACATGATGGTTGATTGAGAATG
CATTGTTGTTTAAGATTGTTGTATCACTAGTAACATTGGATATTGTAGC
AATTTTTTTGAATTTTGTAGTTTTGTCATTTATATGTCGACAGAATTCA
GGTGATAATACTTCTCTATAAATTGCAGGATCTATTTCGTCATTTTTTT
TAATAATTAACTCTATTGCAACTGTATTGAAAATTGAATCTATAAGATT
TATAACAGAATTAGTGTAATTATCATGTGCAGGTTTCATATCTACGAAA
GCACTCCTATAAAATAGAGATCTCTTGTAATCATACTTATCCAAAAAAA
AAGAAAAAAAAAAAGGTATATATATTGCAGGTTTATATTTTTTAGTTAT
ATTGTAAACTTCAGAATTCTTCTTATCAAGTTTGGAAACCAAATTGGCA
TCTTTTAAACATGTAGGTCCATATGATTCTGTGTTTAGGTAATTATATT
GTAGATGCTCCATGAATTTATCATATCGATCATTGCAATCTTCATATAG
ATTTTTATTGGCATCCAACAGTTTTTCTGTAGTAACATCAGGGTTTGTT
AACAAACTGAAGTTCTCTATATCATCCATTATTGTGGGCCAGTGTTTAC
CAAATAGACAACTAATTTCCTTGTAATCAGCATGTTCAGAGACACTATT
CTTGTAACCAGTTTCAATGAATGGTTCTGAATTATCTGCTAACATCCTA
TCATGTGAAAAAAAAAAAAATTCAGGATACTTCCCATATTTTTCACGTA
GTGAAGAACATAGTTCAATACAGTAAGTAACATCACTGATACGATCTGG
ATCAATGTTGATAAGATTAGTGAGCCGATAATCCCCTGTTTCTATGAAG
CCAGATGGTGAGACATTAAAAACACCAATACTTATACCACAGTTACCTA
TAGCATTTTTATATCTGTGATAAAATACATCCAAATCAGTTTTGGCATT
TCTAACTTTAAGCTCAAGTATATACCTTTCATCATCAATCTGGAAATAT
ACATCTGGTGTTAGGATACTGTTGATCTCAGGATATACTTCTTTGATTG
GCTTATCATAACCCAAAATGTTATAACCAGCAGATTGAAGTAAGTCATT
TACATGCATCATTAATGTGTCATGCCTTGACAATTCTAGCAATCCAATG
ACAGTGACTACTATATCTATCTGACTTGGATCAGGTGACATATACAATA
TTTGCTCAGCAAGGTGGACCAATTCTTTGGGGAAATTAATATCTATTGT
GCACTGCTTATAGACTTTCTCTATATTTTTTTTCTTTGATGTTAAAGTG
TAGTTTTTCAGTGTGGTCCCTGCAATTCGAAGAAATTTAGAAAGCACAT
CAGGTGGCATTGCATTACCTGACCGAATCTTAGTGATCGCATCATTGTA
AATCAAACCTTTTTTTAGCTTCCATAATTGCTTTTGAATTTAAATTGTA
TTTAAGGGAGTTCACTACT.

In some embodiments, the RNA1 can be about 50, to 60, 70, 80, 90, 95, 97, 98, 99, or 100% identical SEQ ID NO: 4.

In some embodiments, the protein encoded by RNA1 has the amino acid sequence:

(SEQ ID NO: 3)
MPPDVLSKFLRIAGTTLKNYTLTSKKKNIEKVYKQCTIDINFPKELVHL
AEQILYMSPDPSQIDIVVTVIGLLELSRHDTLMMHVNDLLQSAGYNILG
YDKPIKEVYPEINSILTPDVYFQIDDERYILELKVRNAKTDLDVFYHRY
KNAIGNCGISIGVFNVSPSGFIETGDYRLTNLINIDPDRISDVTYCIEL
CSSLREKYGKYPEFFFFSHDRMLADNSEPFIETGYKNSVSEHADYKEIS
CLFGKHWPTIMDDIENFSLLTNPDVTTEKLLDANKNLYEDCNDRYDKFM
EHLQYNYLNTESYGPTCLKDANLVSKLDKKNSEVYNITKKYKPAIYIPF
FFSFFLDKYDYKRSLFYRSAFVDMKPAHDNYTNSVINLIDSIFNTVAIE
LIIKKNDEIDPAIYREVLSPEFCRHINDKTTKFKKIATISNVTSDTTIL
NNNAFSINHHVDKHMKDNIIGFKNKHYNEKVLKKDCISVADSEEDIITL
KRYMGEIFNSEYHVGTYANELVSIDSDNMNTHICDLPEVCRTKYLDHLY
SQHNIFKALISLNTVNSHKYRLVQTADPCTILITLPNSDTLKSAPLRYF
TLTIISKKDLVSFEANRLLGVAHELIHGRNYNIILSKVISLDVTRLKLL
NHSFGKYCLIMTYYSNMKKKIKHDIHFMIWILCQFITIASLTVTDTYKN
FIKAVYSDYSNIDNLIDDKLESRPTTLGHAYIIKRCLDGIVKATQQLKV
INQKKRNEEIDDMGELQNTGFNNNLSLKLPISNLSVDNPKEIIHEAFML
FYLGNKGLHGSPQELLNLYHTPYKFEKEYSEMLDKYGTILQETGNNSSM
SISYNVMKKTSMVAYANLFNKKDAVRQSICSELEFDKPVLSLKQFSSTK
SMVSNTKTDYVPAPSRLKDSTDIQILEKIINESIIEDDHQFMQLINSEV
YRINAKRLKEEQKNPDMNVTLLPEIYIETIKGVRFIKIKRHQYTRMVNG
GYIKQSNAKVFDEFYKLTEEYGLLSLKDAYKKMIHDDDLLIRIFYKDQR
TADDREIYTGNAQTRLCLYPIEKTYKAICKHIPGEAITISGDQKQKKLL
EQRLTLIKEKRQKIRDNKDAEIYSVSSDASKWSARDLFLKFIIVISTNP
FLHPDEKWFLTFLCFRYYKRNIVLTDNIFNDMLNLVNVNSANTSYEEMT
NDFKQNYFTVRSNWLQGNLNMISSFVHHCSTLYTEAMLKIFSDHNRLEC
NMTSMVHSDDSTYDFLICTGTKNQQKAINKGIINKTNIGKLIIALITFS
NKFHCITLNEKKTYISTFYKEFLSTIIVGNELFFFYLADLLPLTSDTSY
ASPMQDLASYSGYINNAFSHACPLNMIKTSVILINHLTVSTYNMQYTSD
KNPRFHIDSSDLPIQIYPRYKLPLNLAGMIPYYAADAFNILSDIIEKLN
KFKILQTELIEDMLDVNTIEKYLEITRVNSPKVYKYIKSCILCMDYTQY
ERDDADPYNIIDYDLSQKSIINVVSLNKGLRLKKTYTYKKYLEHESEIR
LKAAIHPEWCVKKPTDPDLIKANILQDYTNPNFRDGLIFSTPAIDYGRR
IIGSNKNMYTISSHVMEKDKAKTIGTVYKDLSSKIDEVTLTANDLQRYL
SLYLLSDKKILMAMQVFYSKVETVTMARPSYTKVIQPRSIYAEDFGKYS
NTSLIQSLLTSRYCIINSIDPKSDKFIDICDYVLKRIGDTKIYENQEDI
DDDYIAYYKFKYPNSPDIAIDVEPITDLGEVGFMAYKNKLKFMSLLVRY
YNDIKRTLNNPSFNIPNYPSPSSLIMTIDSLLKKDEISTKVYLSNSKST
KYDEYLLTRYGMYAYNDFHIKYKLGYKIKIASSGKIASNLQTYRDTYEP
LAFITKLIARNLNLYNRLKEHPEFMSGHYNSHELLNVLQNGKDINSSAL
LFMMGKIEFPRFITSLITDHRVYNHWLIPTNSEANDKNASLAYYMCQGN
VMKVRTFTQNNVVVFSMMYYKYLRTDYGALESIKKKIASDYVELLRISK
ITSNITPTERYHVFNINEYGRYTNHFGDRVFNVCPIHYAKIIEVQPSYI
EVDGNVKLILTITTNHEELAFDVRLRTYIDDEYYLNCLIDNLHIERNYI
LGYLCETNLFHKYPDYLKPVLIDLGPNQLISLMTGYTEFDIVTERINPQ
KFGNLIFFSNYFHEHGNSYMGNLCECVQLIALGQNIDVMASKDPDRFIN
SCNKIKLGETCADKVIKNYRANEIAPYHRLFITIMKYRHQSDPIEKIIL
FIILIFKFYIHDFIDMNDELEF.

In some embodiments, the protein encoded by RNA1 can be about 50, to 60, 70, 80, 90, 95, 97, 98, 99, or 100% identical SEQ ID NO: 3.

In some embodiments, the RNA2 has the nucleic acid sequence:

(SEQ ID NO: 6)
AGTAGTGTTCTCCTCATATAAACGCAGAAATTGACAAAAGCTTGAAAAC
AATTAATTGATGAGAATATATCTCAGTTCAGCAGATGTTACTCCATTAT
GAAATGAAGCAACACAATTGGTAATTTGAGGTAATGTTACAAATCACCA
TTGCAGGTTGTTGCATTATAACTCTGCAGCGTGCTGAACAAGGGTGGAC
CATTCCACAAAGGTATAAAAAAGCTTTGGAAAGAAAGGGTAGAGCCAAA
CTTGATTTTTGACTATATATCTGGATCGCCTATTATAATAGACTCAATC
ACTTCTTCTTCACTTATATCATTCTTTTTGTTATATGATAATTTTCTCT
TCTTTTTCAATAATAGGAAATGCTTGATGGTATGTTTACTAATACTAAT
TAACATATATACCACCATTATTACCAAAATTATGCTAAGGATCAATGTT
TTGAAGTTGGTAAGTAAATTAGGAATATGTTTCAATAGGTTAAAATCAT
ATGTAGCACTGCCATGCACTTCATTACTTGTCTTGTAATAGTCAGTGTC
CTTACTATGCTCTAATCTGAATGTGCCACTATACTTATTACACTTGATG
AAAACTTCTTCCCTATCATAAAAAGAGTGCAATGTAAGTGTATTACTAC
TAGAGTCTACGAAATATTCATAACTTACTTTTCCAACATCACATTTGAT
TTGTGCACACTTAGTATTTATATTAAACTTTACCACTATCTCTATACCG
AGTTGACAATCATAACAACCATTGACATTCACTGATATGACATCAGCAG
GTTTTTCACAGAAGTCACCTATTAGTTTACCAATGTTTGGTATGCCCAT
AGAAATCATTCCAAATTCAAATGGTCGTATTATTGTTTGATTCTGATAG
ACAAAATCAGTGGACTTCAAGCTATTCATATCAGTGTTACCAGGGTCAG
TGCAATGGACTAATATTACTTCTCTATCATGTGTCATAGGATCTTTCAC
CATAGGTGAATTCAGTACATACATTTGTCCATCTTTTTTATAGTTCGGG
CCAAAACAACCATAGCTTGGTTGATCACAGAATTGTCCATAAAACACTT
TGTGGTCAGAAATAAGCATTTCATTAGATTCTACTAAGATAGGAGCTAA
AGGTTTAACATAATATGGGAGATGAATATACTTTGAGAACTCTCTTATT
TCAATAACTTCAGTCTTATTACCATGCTTAATTTGGATATCAATATAAG
GAGATACTTTAGCTGTAGTAGCTTTGGTGCCAATATAATGTAATCTTGA
TGTACATGCACCACAGACAGTTGCAGTAGTTGTTAAACAGCTGAAGCCA
TCATGAACCTTTTTAAGATACCATGTATTATCATTTGTGGTCTTCTTTT
TGAGATCTGCTAAGCATTCAACGGCTCCATCACAACTATATGTTACTTC
TTCTATCTTATGTGACACTGGTATATAAATATCAGATATATTGATAACT
TCCATTACCAGATGTGAATTTAGTATAGTAATAGTATATAGATAACCAT
TCACATCAAAATCCTGTTGATAATGCTCTTTATCTAAGACTTGTATTTC
AGTAACATTACTGTGTAAATAATAATTATACTGATTGTCATCTGCTTTA
GCAATAGTTGTTACAACTGATAAGACTAACAAAATGTAGAATAAAAAGT
TGTAATCTGTTTTATTCTTGAAACTTGGAGTTGGACAATCTAGATGGGA
GAACAAATATTCAGAATCACATCTACTACACTTCCATAACCTTTTAGGA
AAGATCTTGTGCAGAGCTTTGTTAAGTAATGCTATGATACACAAAACAG
GTGTCTTTGATATCACTAACATCAACCAAACAAACAGACTTACTATCAC
TTTCCAGGCTTTGTTCAGATAAGTTTCCCACACATAAGACTTATGTGCT
ATGATAGAAGGTTTGGTAGATATATATCGATCCCCATTACAGATAGTGA
CTGTCACTTGTTCCATGTAGCTGCAAGTTTCATTAAACAGGTGAGAATT
AACAGGTTTGATTTGACAACCTGGAAAACAGACAACAAATTCCTTTGCC
AGCTTTTCAATGTTTGGTGTACATGTGCACACATCTACTTCAGCATGGA
AATGGTTTTTCACTTCTTTTACAAATGTCCCACAGCTAAGTAGCAGAAC
TCCAATCGCAACTAGGGTGTTTCTCAGACTCATCTTCATTTTTTTCAAG
AGTTTTTCAAGTTGACTAGTTTTATGAGGAGTTCACTACT.

some embodiments, the RNA2 can be about 50, to 60, 70, 80, 90, 95, 97, 98, 99, or 100% identical SEQ ID NO: 6.

In some embodiments, the protein encoded by RNA2 has the amino acid sequence: MKMSLRNTLVAIGVLLLSCGTFVKEVKNHFHAEVDVCTCTPNIEKLAKEFVVCFPGC QIKPVNSHLFNETCSYMEQVTVTICNGDRYISTKPSIIAHKSYVWETYLNKAWKVIVS LFVWLMLVISKTPVLCIIALLNKALHKIFPKRLWKCSRCDSEYLFSHLDCPTPSFKNKT DYNFLFYILLVLSVVTTIAKADDNQYNYYLHSNVTEIQVLDKEHYQQDFDVNGYLYTI TILNSHLVMEVINISDIYIPVSHKIEEVTYSCDGAVECLADLKKKTTNDNTWYLKKVHD GFSCLTTTATVCGACTSRLHYIGTKATTAKVSPYIDIQIKHGNKTEVIElREFSKYIHLP YYVKPLAPILVESNEMLISDHKVFYGQFCDQPSYGCFGPNYKKDGQMYVLNSPMVK DPMTHDREVILVHCTDPGNTDMNSLKSTDFVYQNQTIIRPFEFGMISMGIPNIGKLIG DFCEKPADVISVNVNGCYDCQLGIEIVVKFNINTKCAQIKCDVGKVSYEYFVDSSSNT LTLHSFYDREEVFIKCNKYSGTFRLEHSKDTDYYKTSNEVHGSATYDFNLLKHIPNLL TNFKTLILSIILVIMVVYMLISISKHTIKHFLLLKKKRKLSYNKKNDISEEEVIESIIIGDPDI (SEQ ID NO: 5). In some embodiments, the protein encoded by RNA2 can be about 50, to 60, 70, 80, 90, 95, 97, 98, 99, or 100% identical SEQ ID NO: 5.

In some embodiments, the RNA3 has the nucleic acid sequence:

(SEQ ID NO: 8)
AGTAGTGTTCTCCCATAATTTATCTAAGCTAACGAAAAACTTTTTAAAA
ACTCAATATATTGGTTTCTAAAGCCTAATAGCGTTTATTTATTGATTTA
TGAAATATATAAACGGATAGAGGAGTTTTATATTTACATCTATTTACAA
CTTACTAACTAGGTGGAACATCTCTTTTTATTATAAACAATCTAGTACA
TATATTAGTTAAGCTACATAAAATATTAGTAGATATATATTTAGATTTG
TAGCTTATTGAAACTTATTATAGACTAGTTACTACTTATCAATCATTAT
ATTTAATACAATCATCTTTTTTGTTTATTTTTTTTGCTTTTTATCGCCT
TTTCTTCTTTTTTTTGTTGTTTTCCATTTTTTATTTTTTTTGATTTTTT
TTGTTTTTTTGTTTTTTTTGTTGTTTTTTGTTTTTTTATTAATATAATA
TTCATATTTAATAATTACTTATATACACCTTCAGTTTTATAAAAAACTG
ATATTTATTGTGCACCTCTATCAGCAGCTAAAGCAGGAGCAAAGTTCTT
GATCAGTTCTTTGAACTTGCCTATAGCTTCATCATTCCTCTTTGATTTG
CTTGTTGTACCAATGTCTCTAGCAAGTTCAACATATGCAAGGGCGAATT
CTTCTCTTCCAATGAGACTTATAGCATCATCGAAAGGAGTAGTCTCTAG
CTTGTGAATACGATTCATCTTCATGACTAAAGAATTGACAATGTCACTG
TCAGTCATGTCATCAGGAATATTCAAGTTTTTTCGGAATTCTAAACGAA
TAAGTGTATAAGCCAACACTTCAGCTGGATAGAGCTCATACAAAAACTC
ATACCCTGGTACAATCATCCAATAATAAGGATTTTTTGAGTCCATTCCC
ATTTGTCCTGCCAGCCTGTTAACAACTGTTTCATCTGGTGCAGTTCTTG
ATGAAACAGTAGTAGGTACATACTTCTTAAGAGTCCAGTCAAACTCTTC
TTTGAACACATGCTTTAGAACACCAGCTGATAAAATTGCACATGCTTTG
TTAAACGAAACAACATTTAGAACATCAGACTCATTCAAGTCTTTAACAA
TTGTCAGAGAATCCTTGTTTGATGTTCTTATAAACACATTTCTTATTTG
AAGCTGCTCCTTGATTTCCAGGGACCTAGAAAGATAAGAAACAGCTATG
CCAACATTACAGAAGTTTCTGTAAGGTGCTAGACTAAAATTGTTGGGAC
TTTGAATCTCTGAAGTAAAAGGTGTAGGTTCAATATAAACTGGGTCCAA
TTCTGAACTTTCAGGCTTTACTAGCTTCTTATTTTGAACATTTGTCAAC
TTGAGGACCCGAAGCTTCTGATCAGCTCCGATGATAACAGTATCGGCTG
CAAGAGTGTTGGATGTGCTTGTTCCAGGATTCTCGATCTTCGAAGGCTT
CTTGAACTCATTCTTTGGTGCCATTGTAGTATTCTCTAAAAACGCTGTT
TTAATGTGAACTCCAAGTATTAGATTTTTAAGAAAAGTTTATCGATCGA
TTGATAATTATGGGAGTTCACTACT.

some embodiments, the RNA3 can be about 50, to 60, 70, 80, 90, 95, 97, 98, 99, or 100% identical SEQ ID NO: 8.

In some embodiments, the protein encoded by RNA3 has the amino acid sequence:

(SEQ ID NO: 7)
MAPKNEFKKPSKIENPGTSTSNTLAADTVIIGADQKLRVLKLTNVQNKK
LVKPESSELDPVYIEPTPFTSEIQSPNNFSLAPYRNFCNVGIAVSYLSR
SLEIKEQLQIRNVFIRTSNKDSLTIVKDLNESDVLNVVSFNKACAILSA
GVLKHVFKEEFDVVTLKKYVPTTVSSRTAPDETVVNRLAGQMGMDSKNP
YYWMIVPGYEFLYELYPAEVLAYTLIRLEFRKNLNIPDDMTDSDIVNSL
VMKMNRIHKLETTPFDDAISLIGREEFALAYVELARDIGTTSKSKRNDE
AIGKFKELIKNFAPALAADRGAQ.

In some embodiments, the protein encoded by RNA3 can be about 50, to 60, 70, 80, 90, 95, 97, 98, 99, or 100% identical SEQ ID NO: 7.

In some embodiments, the RNA4 has the nucleic acid sequence:

(SEQ ID NO: 10)
AGTAGTGTTCTCCTTACATATCAAATCGATCTACACAAAATTTCTTAAA
CAAACAATAAGTTAAGTGATGGAATCTAGACTAGCATTTCCCAAATTTA
TTTCCATTTGTATATGTCGGTACTCTATTTGGGTTGCAAGCAACCTCTT
TTTTTGGTTTTTTGATTTTATGTTTTTTTTGTATTTTTTTACTTTTTTT
GGAAGCTTAAACAACTTAAAGATAACTATATTGCTTAAAACACTTTATT
TACATACATAGCTATATAGATTTAAATTTTAAAACTAGTTTTAAAATAT
GCTCTTGACACTGCAACTTTTTTACAAACTTAGTAGGCTTCTTAATTTT
ACTAACGGTGCTTAATAAGCTTGTACATCTTAGTTGGCTTCACCAATTT
TAATGTAGCCGTAGTCCATCTCTTGAGGGATATTTTCAGCTAAATTGTG
CATTCTTAATACAGCTGCCAGTTTAGTTTTTGCAGCATTTATAGCATTT
AGATCAATTCCTGACAGTGCAGAGCTTAATTCTGCTTTTGCTTCAGAGA
TTTCGTCCTGAATCAATCTAAGCTTAATAGCTTCTTCTCTTTTCTCACT
ATCTATCAACAGCTGTGTTGATGTGTTTGTATCTTCTTTGAGCTTGGCC
CTAACCTTGTTAAGACCTTGATCACTTCCCCAAATAAGTTGGTTTGAGA
TCTCATTTAGCTGCAGCAAAGCCTCTTTCCTTTCATTGTATTTTCTGTC
AAGATAATTTTTTACTTTCAAAAAATTACTCTTTCCTGACATCATTGAA
ACCTCTGGTGGAAGACTTAGAGGGAAAGTGAAAACATTCCATTGAATAT
TCTCATAGACACCATTCTCATCTGGGAGAGTCTTCCAAGACAAGTCTAT
GAATCCAGCTGTAGATTGAGTTTCACCCATCCTAGTATCAGGGAATGAT
ATATAAAACTTGATTTTATTGATATCTTTTGTATCAGTTGCAAAATCAA
GTGAGCCTGTCACGAATTGTTGGAAATTTGGATCAAATGGTACAGCAGT
TATAACAGTTCCCATAAGATCAAGATTAAGCTTCCCCTGAGCCAACAAT
TCTTTTTTTCCTTGTTTGATACCATCATCCCTAAATCTTTCATCCATCA
ATATGAGTGTTGCCATCTCATTAAATCTTTTAGAAGTTGGAGTCCAAAA
TAAAGCTACACTTGCAATCCTTGTCATCGGTTGTTTTAGATGCATATAG
AATTTATACATCTCATTATACACAGATATAGGTAATAGCTGTGTTTTAG
CTTCCAATGTTAACTTTGTTTTAACTCCCATCTTCTTAAAAGTATTCCA
GGTTAGCTCAGTGATGGATTCAATCCCCTCAAATTCCCCAGTTGTCACA
TCAAAGTTGCTGATCTTCGTAGGTTTCATGCTATCAACATTAATAGCCA
TTGCAATCAGAAAAAACAGTATTGAAGAAAAAGCCATCGTACTTAGGTG
TTCTCACTCACAGATTTTAGTTTTTTTAAGAGTTTTTGAAAACAGCTTT
AATTTGTAAGGAGTTCACTACT.

some embodiments, the RNA4 can be about 50, to 60, 70, 80, 90, 95, 97, 98, 99, or 100% identical SEQ ID NO: 10.

In some embodiments, the protein encoded by RNA4 has the amino acid sequence:

(SEQ ID NO: 9)
MAFSSILFFLIAMAINVDSMKPTKISNFDVTTGEFEGIESITELTWNTF
KKMGVKTKLTLEAKTQLLPISVYNEMYKFYMHLKQPMTRIASVALFVVT
PTSKRFNEMATLILMDERFRDDGIKQGKKELLAQGKLNLDLMGTVITAV
PFDPNFQQFVTGSLDFATDTKDINKIKFYISFPDTRMGETQSTAGFIDL
SWKTLPDENGVYENIQWNVFTFPLSLPPEVSMMSGKSNFLKVKNYLDRK
YNERKEALLQLNEISNQLIWGSDQGLNKVRAKLKEDTNTSTQLLIDSEK
REEAIKLRLIQDEISEAKAELSSALSGIDLNAINAAKTKLAAVLRMHNL
AENIPQEMDYGYIKIGEAN.

In some embodiments, the protein encoded by RNA4 can be about 50, to 60, 70, 80, 90, 95, 97, 98, 99, or 100% identical SEQ ID NO: 9.

In some embodiments, the RNAS has the nucleic acid sequence:

(SEQ ID NO: 12)
AGTAGTGTTCTCCCACAAAAATATCAAATTCAATGAAAACTTTCTTAAG
CTAACCAAGTGGCCTAAATCAACAACAAGACGTGCAAGTTAATTAAATA
ACCAAGCTAATCATACAATTGTATAACAAGTTCCAAATTGCTACCTATT
TCATTGTTTAAGATGAAATATATTGCCATCATTGGAACAAATATATTGT
AACTCTATTGAGTCCAATTTACTTTCAAAAAGATTTGCATGCAACCATC
CTTAAGAGATATTCGATTCTGATGTAAGAAAAGGAGTCTGTTTCTTAAG
TGACTGACATTAACTTTTGCGAATTCCCTTGCTCTCTCTAGTACTTCAA
CTGTCCCTATATTTTCACTGTAACCTGTTCTAATAGCTGAAACTTTGGC
CATGTATAAATATAAACAGATATAATGTAATGTTAGGTGAGACATCTTT
AAATCACTGTTTTTCAGTTTATATAAGTTGTAAACCTCTTTTTTGTAAC
TAGATTTGAATGGGTAATTAGGTATGTAATCTATGTCATCAATTTTTGT
ATCATCTGATATATTCTTTTTCATGGAGCTATTGATATTGTTGACAACT
GTTTTAATACTACCAGGTAAGCCATAATAAAACATATCTTCAATCTCAT
TATCACCAGGCAAATACTTGATCTTTTCATCAACAGCTTTATTCAACGC
TATATCATTCAAAAGAATTTCTTTATCAATATATTCGATAATTGGTTTT
TTCAATACATCTACTTCTGAGTTCTCTATCCTCACTTCTCTTAGTCTTA
TTGTAACATCCTCATAAATTGCTGTTTTAACATCTAAAGGGAAGAACTC
ACCATACATAAAATCTGATTCTTTGATTTTGTTGCAATCAAATGGTATA
TCATAATCTAACATCTTTTTACAGTATTGGATATGTTCTTTGACATATT
TAAATACATGTATGTTATAAGTCCTAAGCATGATATCAAGACTTGGAAT
GATCTCATTAGATAAGTGTTTGTTTTCATCTATAAAAGAATAAACTGAT
TTCTCATATCTTGAATTACATACAATCCTATAATAAAATGACTTCATTC
TGAGGTAGTGACTTATGATCAATTCTTTTTGTGCGAGATATGTAGAGCC
TATTATATTAGCCCAATTTTTCTTTGTTTCATTTATTATCCATTGTTTA
TCAATGATTTTAACCATTATAAAGTAATTTTTGACTGCTGCTGCATTTT
TTATCACTGCTGTTACCATTTCTAAGTTTTCATAATTTTCTATAACATA
GTTATACACAATGCTTTTTAATGCATTAACAACATCCTCAGGTAGTACT
TCAAGCCTCACAGTCCCTCTGTTTAGAACTGTAGCAATAACAGTTGGCA
TTTCACCTCTATTAATCTCTATGTAGCCGATAGATGAATTACATGGTAT
GCGAGTGTACTCACCATAAAACAATTTCTTCTCAACACAGTTGCAGCTG
TTAAAATTTTCTCGAATCAGCTTGTATATAGGTCCATAGAAGTCACCGA
GACATGGCAGCTTGGGTAGTTTCTCATGGTTACTAAAGTCAACTCTTTC
AGGGTCTGGACGAACGTAAGGGATGATTTTTTCCATCACTGGTGCAAGC
TTTAAAAGAGTTTTTTGTTAATCGAAATTATTGTGGGAGTTCACTACT.

some embodiments, the RNA5 can be about 50, to 60, 70, 80, 90, 95, 97, 98, 99, or 100% identical SEQ ID NO: 12.

In some embodiments, the protein encoded by RNA5 has the amino acid sequence:

(SEQ ID NO: 11)
MEKIIPYVRPDPERVDFSNHEKLPKLPCLGDFYGPIYKLIRENFNSCNC
VEKKLFYGEYTRIPCNSSIGYIEINRGEMPTVIATVLNRGTVRLEVLPE
DVVNALKSIVYNYVIENYENLEMVTAVIKNAAAVKNYFIMVKIIDKQWI
INETKKNWANIIGSTYLAQKELIISHYLRMKSFYYRIVCNSRYEKSVYS
FIDENKHLSNEIIPSLDIMLRTYNIHVFKYVKEHIQYCKKMLDYDIPFD
CNKIKESDFMYGEFFPLDVKTAIYEDVTIRLREVRIENSEVDVLKKPII
EYIDKEILLNDIALNKAVDEKIKYLPGDNEIEDMFYYGLPGSIKTVVNN
INSSMKKNISDDTKIDDIDYIPNYPFKSSYKKEVYNLYKLKNSDLKMSH
LTLHYICLYLYMAKVSAIRTGYSENIGTVEVLERAREFAKVNVSHLRNR
LLFLHQNRISLKDGCMQIFLKVNVVTQ.

In some embodiments, the protein encoded by RNA5 can be about 50, to 60, 70, 80, 90, 95, 97, 98, 99, or 100% identical SEQ ID NO: 11.

In some embodiments, the RNA6 has the nucleic acid sequence:

(SEQ ID NO: 15)
AGTAGTGTTCTCCCTATAAACTTCAGCAGCTTTCAAAAAACTTTCTTAA
ACTATAAAAATTTGGTGATTTGGTTCTAAAAACGTTCGTATTGCCATTT
ATAAAAATTGTAGCAATATATCTTTGTTCAAATTCCAAATTTAATATCG
GTTATTGGTCATATTATACTTTTTCAAGCATAACATAACGTTTTTTTTG
TTAGATATTTACTGTTTCAAAAATATTTTACAGGCAGCAAATATCTGAT
TTTTTGTTTTTTTGTTTTTTTTGTTTTTTTTGTTGAGAAATGTTTCCTC
TTTTTATGCTTTTTTTTGGTCTTTTGTGTTTTTATCTGGACTATTATAT
TCAGAATATTGTTATCTATGCCATTTGAAGAAGATTTTTTGAAGAAATA
ATAAAGCTTGTGGTAACTATTAATATATCTATTATTTATTGGTTACTAT
ATTATAATATCCATCTGATCTTTGTTTTGATTTTTTTTAGTTTATAGAC
TCTTTTGTGCTTTTTTTCGTCATTTTTCTTTTTTTATATATAAAACATA
ATAAAACTTACAAGTGCCTGTAGGCTATTGTAATTAAGTGACAAGCTTA
TTGAACAATACCAATACTTATTGCATCTTATCAATGTCTTGGTTTTAAT
GATTATGATACACAGTTTCTTCAAGCTTAGTATCATATTTATCATCAAT
AACCATGACTGATGAAGCACTAGCATTATTTATAGTATTATTAATCTGA
TGTGTTTCCAATGGAAGTTTATTGTTCAATCTGCCAGTGGTATAAGCAA
TAGCATGAACATGAAACAAAAAAGCAACAGTTGATTCTAAATAATTCAC
ATCTTCTTGTTGTTGAGATTGTGCCTTAATTATTGTGTCTTCTGGATCA
TAACCTAGGACCACTTGTTTTACAATTGCTTTCACGATATTTGAAGACA
TCAATAAGTACTGTGAATGGTCTGGTATATATGGAAATATTCTTTCACC
TCTCACAAGAGTACCAAATATGTGGAAATCATGATGGATGCTTTCAGGC
AATACACTTGTCAATCCAAACAAAGAACATGTGATTTGTAAATACCTCA
AAAACTTTCTAAGTGATTTCTTTTGTATCTTCATCCCGCTTATTTCAAT
TTGCTGTGTTTGATAGTTGATCTCCGAAAATTCAAGCTGATTTTTTAGA
TATGCATCATGTAGAACAATCACCTCACTATACTCTTCATCGTTGTCTA
GGATCTCAATCATTGAGTTGAAAATAGCAGTCTCCAATTCACAGAAATG
ATCAGAGAATTTGTTATTGACAAGAGCTTTGTATGTTTTCATGAACGTC
TTTTGCTCCATGGTTGACCTAAATATGATTTGCTTTAAGAGTTTTTCAA
GTCGCTTGGTTTTATAGGGAGTTCACTACT.

some embodiments, the RNA6 can be about 50, to 60, 70, 80, 90, 95, 97, 98, 99, or 100% identical SEQ ID NO: 15.

In some embodiments, the protein encoded by RNA6 has the amino acid sequence: MLVLHQSWLLMINMILSLKKLCIIIIKTKTLIRCNKYWYCSISL (SEQ ID NO: 13). In some embodiments, the protein encoded by RNA6 can be about 50, to 60, 70, 80, 90, 95, 97, 98, 99, or 100% identical SEQ ID NO: 13.

In some embodiments, the protein encoded by RNA6 has the amino acid sequence:

(SEQ ID NO: 14)
MEQKTFMKTYKALVNNKFSDHFCELETAIFNSMIEILDNDEEYSEVIVL
HDAYLKNQLEFSEINYQTQQIEISGMKIQKKSLRKFLRYLQITCSLFGL
TSVLPESIHHDFHIFGTLVRGERIFPYIPDHSQYLLMSSNIVKAIVKQV
VLGYDPEDTIIKAQSQQQEDVNYLESTVAFLFHVHAIAYTTGRLNNKLP
LETHQINNTINNASASSVMVIDDKYDTKLEETVYHNH.

In some embodiments, the protein encoded by RNA6 can be about 50, to 60, 70, 80, 90, 95, 97, 98, 99, or 100% identical SEQ ID NO: 14.

In some embodiments, the RNA7 has the nucleic acid sequence:

(SEQ ID NO: 17)
AGTAGTGTTCTCCCACAAATTAATCAAAAAACTGATAAAAGCTTGAAAA
CTCTAATATAAGTGGAATTAACAATTCATAGATGACTAAATTTTATTCT
AATTCGCTAATTATTTCACTTTTTAAGGTGAAATACTTCGCCATAATTA
GAACTTAATGGAAATAATTATTATTTAAAAGATAGAATACCAATTACAT
ATTGAATTTTAAGCTAATTTGTACACATTCATTTTTGAATTGCTTCCTA
TTTTGATTTAGAAAAAGTAGCCTATTCCTTAGGTAGCTCACATTAGTTT
TAGCGAATAATCTGGCTCTCTCTAAAACAGCTGAATGTGCTGTATAACA
ACCAGTATCAATCTTTAGACTAGCAACTTTAGATATGTATAAAAATAGA
CAAATATAATGAAGAGTCAGATGTGAGACTTTAACAGTATCATTCCTAA
CCTTTAATGCAGTGAATATTTCACGTTTGAAGCTGTTCTTGAATGGAAA
TTGAGGTATATAGTCTATATTGTTTATATCATCTACATCATGAATATTT
TTTTCCAAAGCATCTTCTAGATGATCAATCACAGTTTCTATTGTATCTG
GAATACCATAGTAAAAGGTATCCTCGATTTCATCATCACCTGGTTTATA
TGAGACTGTCTCATCAGTATGTTTCCAAAAAGTAACTTTGTTTAGAACA
ACTACATCATCTACACAATCAATTATTGGTTTCTTCAATACATCTTCTT
CACTTCTATTGACTTTTACTTCTCTTAGTTTAGCTGTCACATCAATATG
AATAGCTGCTAAGACATCAGCTGGAAATTTTTCACCATATATAGAGTCT
GATTCAGAAATTGTAGTGTAATCGAAGGCAAGATTGTAATCTGATTTGT
CTTTACATATCTGTAAGTGACTTTTGACATATTTTATTATCTGTGCATT
ATACAGCTTAAAATCAACATCTAAGCTTGGAAGAACATGATTCAGAAGG
TGTTTATTATCATCCAAGAAACTGTAAACTGATTTCTCAGTATCAGAAT
TTTTCACTATCTTGTAATAGAATGCTGTCATGCTTGTGTAATGACTTAA
AATTACCTCTCTCTGTGAAAGATATGTTGAGCTTAGAATGTTAGCCCAA
TTCCTCTTCACTTCATCTATCATCCATTGTTTGTCAACTATTTTTATCA
TTATAAAGTATCGTCTGACCAACTTATGATGCATTATAACAGCTGACAG
AGTATCTAAGTTGTTAAAATTCTCTATAACATAGTTATATAGAATGCTC
TGAAGTGCTAAACAGACATCTTCAGGTAATGTTTCAACTTCAACTTGAG
TTTTGTTAAACACTGATGCTACAGTGTTTGGTATTTTTTTATGATTAAT
CTCCAAGTAACAGATAGATGAATGTTTCGGAATCTTATTGTGTGAACCA
TAAAAGAATTTTTTCTCAGTACTGACATCAGCATTGAACATAGTCTTGA
TATTGCTATATATAGTTCCATAGAAAGCACCAAGATTGTTAAGCTTACC
TAGCTTTTCATAATTTGAAAAGTCAATGACTTCAGAATCTGAACGCGTG
TAAGGTATGATTGAATCCATCACTTGTTGAGTTTTTTTAAGCTTTTTTT
CAAAATCTATTTAATTGTGGGAGTTCACTACT.

some embodiments, the RNA7 can be about 50, to 60, 70, 80, 90, 95, 97, 98, 99, or 100% identical SEQ ID NO: 17.

In some embodiments, the protein encoded by RNA7 has the amino acid sequence:

(SEQ ID NO: 16)
MDSIIPYTRSDSEVIDFSNYEKLGKLNNLGAFYGTIYSNIKTMFNADVST
EKKFFYGSHNKIPKHSSICYLEINHKKIPNTVASVFNKTQVEVETLPEDV
CLALQSILYNYVIENFNNLDTLSAVIMHHKLVRRYFIMIKIVDKQWMIDE
VKRNWANILSSTYLSQREVILSHYTSMTAFYYKIVKNSDTEKSVYSFLDD
NKHLLNHVLPSLDVDFKLYNAQIIKYVKSHLQICKDKSDYNLAFDYTTIS
ESDSIYGEKFPADVLAAIHIDVTAKLREVKVNRSEEDVLKKPIIDCVDDV
VVLNKVTFWKHTDETVSYKPGDDEIEDTFYYGIPDTIETVIDHLEDALEK
NIHDVDDINNIDYIPQFPFKNSFKREIFTALKVRNDTVKVSHLTLHYICL
FLYISKVASLKIDTGCYTAHSAVLERARLFAKTNVSYLRNRLLFLNQNRK
QFKNECVQISLKFNM.

In some embodiments, the protein encoded by RNA7 can be about 50, to 60, 70, 80, 90, 95, 97, 98, 99, or 100% identical SEQ ID NO: 16.

In aspects, the agRNA1 can be about 50, 60, 70, 80, 90, 95, 97, 98, 99, or 100% identical to the complementary polynucleotide to RNA1 (SEQ ID NO: 4). In aspects, the agRNA1 can be about 50, to 60, 70, 80, 90, 95, 97, 98, 99, or 100% identical to the complementary polynucleotide to RNA1 (SEQ ID NO: 4).

In aspects, the agRNA2 can be about 50, 60, 70, 80, 90, 95, 97, 98, 99, or 100% identical to the complementary polynucleotide to RNA2 (SEQ ID NO: 6). In aspects, the agRNA2 can be about 50, to 60, 70, 80, 90, 95, 97, 98, 99, or 100% identical to the complementary polynucleotide to RNA2 (SEQ ID NO: 6).

In aspects, the agRNA3 can be about 50, 60, 70, 80, 90, 95, 97, 98, 99, or 100% identical to the complementary polynucleotide to RNA3 (SEQ ID NO: 8). In aspects, the agRNA3 can be about 50, to 60, 70, 80, 90, 95, 97, 98, 99, or 100% identical to the complementary polynucleotide to RNA3 (SEQ ID NO: 8).

In aspects, the agRNA4 can be about 50, 60, 70, 80, 90, 95, 97, 98, 99, or 100% identical to the complementary polynucleotide to RNA4 (SEQ ID NO: 10). In aspects, the agRNA4 can be about 50, to 60, 70, 80, 90, 95, 97, 98, 99, or 100% identical to the complementary polynucleotide to RNA4 (SEQ ID NO: 10).

In aspects, the agRNA5 can be about 50, 60, 70, 80, 90, 95, 97, 98, 99, or 100% identical to the complementary polynucleotide to RNAS (SEQ ID NO: 12). In aspects, the agRNA5 can be about 50, to 60, 70, 80, 90, 95, 97, 98, 99, or 100% identical to the complementary polynucleotide to RNAS (SEQ ID NO: 12).

In aspects, the agRNA6 can be about 50, 60, 70, 80, 90, 95, 97, 98, 99, or 100% identical to the complementary polynucleotide to RNA6 (SEQ ID NO: 15). In aspects, the agRNA6 can be about 50, to 60, 70, 80, 90, 95, 97, 98, 99, or 100% identical to the complementary polynucleotide to RNA6 (SEQ ID NO: 15).

In aspects, the agRNA7 can be about 50, 60, 70, 80, 90, 95, 97, 98, 99, or 100% identical to the complementary polynucleotide to RNA7 (SEQ ID NO: 17, Appendix B). In aspects, the agRNA7 can be about 50, to 60, 70, 80, 90, 95, 97, 98, 99, or 100% identical to the complementary polynucleotide to RNA7 (SEQ ID NO: 17, Appendix B).

In some aspects the RRV RNA or RRV agRNA can be directly fused to or indirectly linked (or operatively coupled) to an RNA that encodes a polypeptide or its antigenomic sequence (or cDNA). The polypeptide can be any desired polypeptide including, but not limited to a reporter protein (e.g. a fluorescent protein or other selectable marker, such as those that confer a selectable phenotype in plant cells). For example, the selectable marker can encode a protein that confers biocide resistance, antibiotic resistance (e.g., resistance to kanamycin, G418, bleomycin, hygromycin), or herbicide resistance (e.g., resistance to chlorosulfuron or Basta). Thus, the presence of the selectable phenotype indicates the successful transformation of the host cell. Exemplary selectable markers include, but are not limited to, the beta-glucuronidase, green fluorescent protein, or iLOV fluorescent protein. As is described elsewhere herein, other polypeptides can be included as the infectious RRV polynucleotides and vectors can be used as a gene delivery system as is demonstrated by delivery of the fluorescent proteins herein. Examples of reporter gene/agRNA constructs are provided below (SEQ ID NOs: 18, 19, and 20).

RRV_agRNA4_Fused GFP:
(SEQ ID NO: 18)
AGTAGTGAACTCCTTACAAATTAAAGCTGTTTTCAAAAACTCTTAAAAAAACTAAA
ATCTGTGAGTGAGAACACCTAAGTACGATGGCTTTTTCTTCAATACTGTTTTTTCT
GATTGCAATGGCTATTAATGTTGATAGCATGAAACCTACGAAGATCAGCAACTTT
GATGTGACAACTGGGGAATTTGAGGGGATTGAATCCATCACTGAGCTAACCTGG
AATACTTTTAAGAAGATGGGAGTTAAAACAAAGTTAACATTGGAAGCTAAAACAC
AGCTATTACCTATATCTGTGTATAATGAGATGTATAAATTCTATATGCATCTAAAA
CAACCGATGACAAGGATTGCAAGTGTAGCTTTATTTTGGACTCCAACTTCTAAAA
GATTTAATGAGATGGCAACACTCATATTGATGGATGAAAGATTTAGGGATGATG
GTATCAAACAAGGAAAAAAAGAATTGTTGGCTCAGGGGAAGCTTAATCTTGATCT
TATGGGAACTGTTATAACTGCTGTACCATTTGATCCAAATTTCCAACAATTCGTG
ACAGGCTCACTTGATTTTGCAACTGATACAAAAGATATCAATAAAATCAAGTTTTA
TATATCATTCCCTGATACTAGGATGGGTGAAACTCAATCTACAGCTGGATTCATA
GACTTGTCTTGGAAGACTCTCCCAGATGAGAATGGTGTCTATGAGAATATTCAAT
GGAATGTTTTCACTTTCCCTCTAAGTCTTCCACCAGAGGTTTCAATGATGTCAGG
AAAGAGTAATTTTTTGAAAGTAAAAAATTATCTTGACAGAAAATACAATGAAAGGA
AAGAGGCTTTGCTGCAGCTAAATGAGATCTCAAACCAACTTATTTGGGGAAGTG
ATCAAGGTCTTAACAAGGTTAGGGCCAAGCTCAAAGAAGATACAAACACATCAA
CACAGCTGTTGATAGATAGTGAGAAAAGAGAAGAAGCTATTAAGCTTAGATTGAT
TCAGGACGAAATCTCTGAAGCAAAAGCAGAATTAAGCTCTGCACTGTCAGGAAT
TGATCTAAATGCTATAAATGCTGCAAAAACTAAACTGGCAGCTGTATTAAGAATG
CACAATTTAGCTGAAAATATCCCTCAAGAGATGGACTACGGCTACATTAAAATTG
GTGAAGCCAACaggcctatggtttctaagggtgaggaactcttcaccggtgttgttcctatcctcgtggaactcga
tggtgatgttaacggacacaagttctctgtgtctggtgaaggtgagggtgatgcaacttacggaaagctcaccctcaag
ttcatctgtaccactggaaagctccctgtgccttggcctactcttgttactactctcacttacggtgtgcagtgcttctcaaga
taccctgatcacatgaagcagcacgatttcttcaagtctgctatgcctgagggatacgtgcaagagaggaccatcttctt
caaggatgatggaaactacaagaccagggctgaggtgaagttcgaaggtgatactctcgtgaacaggatcgagctt
aagggaatcgatttcaaagaggatggtaacatccttggacacaagctcgagtacaactacaactcacacaacgtgta
catcatggcagataagcagaagaacggaatcaaggttaacttcaagatcaggcacaacatcgaggatggttctgtg
cagctcgctgatcattaccagcagaacactcctatcggagatggacctgttctcctccctgataaccactacctttctacc
cagtctaagctctctaaagatcctaacgagaagagggatcacatggtgctcctcgagtttgttacagccgctggaatca
ccctcggaatggatgagctttacaagtgacccgggGATGTACAAGCTTATTAAGCACCGTTAGTA
AAATTAAGAAGCCTACTAAGTTTGTAAAAAAGTTGCAGTGTCAAGAGCATATTTT
AAAACTAGTTTTAAAATTTAAATCTATATAGCTATGTATGTAAATAAAGTGTTTTAA
GCAATATAGTTATCTTTAAGTTGTTTAAGCTTCCAAAAAAAGTAAAAAAATACAAA
AAAAACATAAAATCAAAAAACCAAAAAAAGAGGTTGCTTGCAACCCAAATAGAGT
ACCGACATATACAAATGGAAATAAATTTGGGAAATGCTAGTCTAGATTCCATCAC
TTAACTTATTGTTTGTTTAAGAAATTTTGTGTAGATCGATTTGATATGTAAGGAGA
ACACTACT.
RRV_agRNA4_2a_GFP:
(SEQ ID NO: 19)
AGTAGTGAACTCCTTACAAATTAAAGCTGTTTTCAAAAACTCTTAAAAAAACTAAA
ATCTGTGAGTGAGAACACCTAAGTACGATGGCTTTTTCTTCAATACTGTTTTTTCT
GATTGCAATGGCTATTAATGTTGATAGCATGAAACCTACGAAGATCAGCAACTTT
GATGTGACAACTGGGGAATTTGAGGGGATTGAATCCATCACTGAGCTAACCTGG
AATACTTTTAAGAAGATGGGAGTTAAAACAAAGTTAACATTGGAAGCTAAAACAC
AGCTATTACCTATATCTGTGTATAATGAGATGTATAAATTCTATATGCATCTAAAA
CAACCGATGACAAGGATTGCAAGTGTAGCTTTATTTTGGACTCCAACTTCTAAAA
GATTTAATGAGATGGCAACACTCATATTGATGGATGAAAGATTTAGGGATGATG
GTATCAAACAAGGAAAAAAAGAATTGTTGGCTCAGGGGAAGCTTAATCTTGATCT
TATGGGAACTGTTATAACTGCTGTACCATTTGATCCAAATTTCCAACAATTCGTG
ACAGGCTCACTTGATTTTGCAACTGATACAAAAGATATCAATAAAATCAAGTTTTA
TATATCATTCCCTGATACTAGGATGGGTGAAACTCAATCTACAGCTGGATTCATA
GACTTGTCTTGGAAGACTCTCCCAGATGAGAATGGTGTCTATGAGAATATTCAAT
GGAATGTTTTCACTTTCCCTCTAAGTCTTCCACCAGAGGTTTCAATGATGTCAGG
AAAGAGTAATTTTTTGAAAGTAAAAAATTATCTTGACAGAAAATACAATGAAAGGA
AAGAGGCTTTGCTGCAGCTAAATGAGATCTCAAACCAACTTATTTGGGGAAGTG
ATCAAGGTCTTAACAAGGTTAGGGCCAAGCTCAAAGAAGATACAAACACATCAA
CACAGCTGTTGATAGATAGTGAGAAAAGAGAAGAAGCTATTAAGCTTAGATTGAT
TCAGGACGAAATCTCTGAAGCAAAAGCAGAATTAAGCTCTGCACTGTCAGGAAT
TGATCTAAATGCTATAAATGCTGCAAAAACTAAACTGGCAGCTGTATTAAGAATG
CACAATTTAGCTGAAAATATCCCTCAAGAGATGGACTACGGCTACATTAAAATTG
GTGAAGCCAACaggcctcagcttctgaactttgatctgctcaagctggcgggcgatgtggaatccaacccagg
cccaatggtttctaagggtgaggaactcttcaccggtgttgttcctatcctcgtggaactcgatggtgatgttaacggaca
caagttctctgtgtctggtgaaggtgagggtgatgcaacttacggaaagctcaccctcaagttcatctgtaccactggaa
agctccctgtgccttggcctactcttgttactactctcacttacggtgtgcagtgcttctcaagataccctgatcacatgaag
cagcacgatttcttcaagtctgctatgcctgagggatacgtgcaagagaggaccatcttcttcaaggatgatggaaact
acaagaccagggctgaggtgaagttcgaaggtgatactctcgtgaacaggatcgagcttaagggaatcgatttcaaa
gaggatggtaacatccttggacacaagctcgagtacaactacaactcacacaacgtgtacatcatggcagataagc
agaagaacggaatcaaggttaacttcaagatcaggcacaacatcgaggatggttctgtgcagctcgctgatcattacc
agcagaacactcctatcggagatggacctgttctcctccctgataaccactacctttctacccagtctaagctctctaaag
atcctaacgagaagagggatcacatggtgctcctcgagtttgttacagccgctggaatcaccctcggaatggatgagc
tttacaagtgacccgggGATGTACAAGCTTATTAAGCACCGTTAGTAAAATTAAGAAGCC
TACTAAGTTTGTAAAAAAGTTGCAGTGTCAAGAGCATATTTTAAAACTAGTTTTAA
AATTTAAATCTATATAGCTATGTATGTAAATAAAGTGTTTTAAGCAATATAGTTATC
TTTAAGTTGTTTAAGCTTCCAAAAAAAGTAAAAAAATACAAAAAAAACATAAAATC
AAAAAACCAAAAAAAGAGGTTGCTTGCAACCCAAATAGAGTACCGACATATACAA
ATGGAAATAAATTTGGGAAATGCTAGTCTAGATTCCATCACTTAACTTATTGTTTG
TTTAAGAAATTTTGTGTAGATCGATTTGATATGTAAGGAGAACACTACT.
RRV_agRNA2_Fused_iLOV:
(SEQ ID NO: 20)
agtagtgaactcctcataaaactagtcaacttgaaaaactcttgaaaaaaatgaagatgagtctgagaaacacccta
gttgcgattggagttctgctacttagctgtgggacatttgtaaaagaagtgaaaaaccatttccatgctgaagtagatgtgt
gcacatgtacaccaaacattgaaaagctggcaaaggaatttgttgtctgttttccaggttgtcaaatcaaacctgttaattc
tcacctgtttaatgaaacttgcagctacatggaacaagtgacagtcactatctgtaatggggatcgatatatatctaccaa
accttctatcatagcacataagtcttatgtgtgggaaacttatctgaacaaagcctggaaagtgatagtaagtctgtttgttt
ggttgatgttagtgatatcaaagacacctgttttgtgtatcatagcattacttaacaaagctctgcacaagatctttcctaaa
aggttatggaagtgtagtagatgtgattctgaatatttgttctcccatctagattgtccaactccaagtttcaagaataaaac
agattacaactttttattctacattttgttagtcttatcagttgtaacaactattgctaaagcagatgacaatcagtataattatt
atttacacagtaatgttactgaaatacaagtcttagataaagagcattatcaacaggattttgatgtgaatggttatctatat
actattactatactaaattcacatctggtaatggaagttatcaatatatctgatatttatataccagtgtcacataagataga
agaagtaacatatagttgtgatggagccgttgaatgcttagcagatctcaaaaagaagaccacaaatgataatacatg
gtatcttaaaaaggttcatgatggcttcagctgtttaacaactactgcaactgtctgtggtgcatgtacatcaagattacatt
atattggcaccaaagctactacagctaaagtatctccttatattgatatccaaattaagcatggtaataagactgaagtta
ttgaaataagagagttctcaaagtatattcatctcccatattatgttaaacctttagctcctatcttagtagaatctaatgaaa
tgcttatttctgaccacaaagtgttttatggacaattctgtgatcaaccaagctatggttgttttggcccgaactataaaaaa
gatggacaaatgtatgtactgaattcacctatggtgaaagatcctatgacacatgatagagaagtaatattagtccattg
cactgaccctggtaacactgatatgaatagcttgaagtccactgattttgtctatcagaatcaaacaataatacgaccatt
tgaatttggaatgatttctatgggcataccaaacattggtaaactaataggtgacttctgtgaaaaacctgctgatgtcata
tcagtgaatgtcaatggttgttatgattgtcaactcggtatagagatagtggtaaagtttaatataaatactaagtgtgcac
aaatcaaatgtgatgttggaaaagtaagttatgaatatttcgtagactctagtagtaatacacttacattgcactctttttatg
atagggaagaagttttcatcaagtgtaataagtatagtggcacattcagattagagcatagtaaggacactgactatta
caagacaagtaatgaagtgcatggcagtgctacatatgattttaacctattgaaacatattcctaatttacttaccaacttc
aaaacattgatccttagcataattttggtaataatggtggtatatatgttaattagtattagtaaacataccatcaagcatttc
ctattattgaaaaagaagagaaaattatcatataacaaaaagaatgatataagtgaagaagaagtgattgagtctatt
ataataggcgatccagatataATGGCTAGCATAGAGAAGAATTTCGTCATCACTGATCCTA
GGCTTCCCGATAATCCCATTATCTTTGCATCAGACGGCTTTCTTGAATTGACAGA
GTATTCGCGCGAGGAAATATTGGGGAGAAATGCCCGGTTTCTTCAGGGGCCAG
AGACAGATCAAGCGACTGTCCAGAAGATAAGAGACGCAATTAGAGATCAGAGG
GAGACTACTGTGCAGTTGATAAACTACACTAAAAGCGGAAAGAAATTCTGGAAC
TTACTCCACCTGCAACCTGTGCGTGATCAGAAGGGAGAGCTTCAATACTTCATC
GGTGTGCAGCTCGATGGAAGTGATCATGTAtagtcaaaaatcaagtttggctctaccctttctttcca
aagcttttttatacctttgtggaatggtccacccttgttcagcacgctgcagagttataatgcaacaacctgcaatggtgatt
tgtaacattacctcaaattaccaattgtgttgcttcatttcataatggagtaacatctgctgaactgagatatattctcatcaat
taattgttttcaagcttttgtcaatttctgcgtttatatgaggagaacactact.

The RRV recombinant polynucleotides described herein can be incorporated into a suitable vector. Thus, described herein are aspects of infectious RRV vectors that can include on or more RRV RNA segments and/or one or more RRV agRNA segments as described above. The RRV RNA and/or agRNA segment(s) can be fused directly to or operatively coupled to one or more regulatory segments (e.g. promoters, enhancers, etc.) and/or one or more other polynucleotides that can encode a polynucleotide as previously described. In some aspects, a TMV omega translational enhancer can be fused directly or operatively linked to the RRV RNA segment, RRV agRNA segment, other regulatory sequence, and/or reporter gene (e.g. GFP or iLOV), or other exogenous gene of interest. In addition to other vectors described elsewhere herein, suitable vectors can include those that are appropriate for plant transformation using agrobacterium. In some aspects, the vector can be based upon a Ti-plasmid or a Ri-plasmid. Such vectors are commercially available and will be appreciated by those of ordinary skill in the art and are within the scope of this disclosure. In some aspects, the vector can be pCB301.

Infectious RRV Agrobacterium and Uses Thereof

Described herein are agrobacterium and populations thereof, wherein at least one agrobacterium can include one or more RRV RNAs (e.g. RRV RNA1, 2, 3, 4, 5, 6, 7 or any combination thereof as described elsewhere herein) and/or one or more RRV agRNA polynucleotide (e.g. RRV agRNA 1, 2, 3, 4, 5, 6, 7, or any combination thereof as described elsewhere herein) or a vector containing one or more RRV RNA polynucleotides and/or one or more RRV agRNA polynucleotides as described elsewhere herein. Suitable techniques for transforming the agrobacterium with the RRV RNA(s), RRV agRNA(s), and/or a vector containing the RRV RNA(s) and/or RRV agRNAs described herein are generally known in the art.

A transformed agrobacterium (also referred to herein as an infectious RRV agrobacterium) or population thereof can be used to make stably genetically modified plants as is described in greater detail below. In other aspects, a formulation containing an infectious RRV agrobacterium or population thereof can be applied to one or more parts of a plant (e.g. leaves, stem, roots, etc.) using any suitable method to allow (e.g. spraying) transient exogenous gene expression of the RRV RNA and/or RRV agRNA in the plants. The formulation can contain 1×10², 1×10³, 1×10⁴, 1×10⁵, 1×10⁶, 1×10⁷, 1×10⁸, 1×10⁹, 1×10¹⁰, 1×10¹¹, 1×10¹² or more transformed agrobacteria suspended in a suitable media. The formulation can contain 1×10², 1×10³, 1×10⁴, 1×10⁵, 1×10⁶, 1×10⁷, 1×10⁸, 1×10⁹, 1×10¹⁰, 1×10¹¹, 1×10¹² or more per mL transformed agrobacteria suspended in a suitable media.

In some aspects, the plant to which the transformed agrobacterium can be applied can be a species or cultivar from the genus Rosa, Arabidopsis (e.g. A. thaliana), Nicotiana (e.g. N. benthamiana), Brassica (e.g. Brassica napus), Fragaria, and/or Rubus.

In some aspects, application of the transformed agrobacteria described herein to a plant can increase the performance characteristic or phenotype of the plant to which it is applied. In some aspects, the plant to which the transformed agrobacteria is applied can have increased growth and/or increased yield (e.g. increased fruit yield, increased flowering, and/or increased seed yield) as compared to a suitable control. In some aspects, the growth rate of the plant to which the transformed agrobacterium is applied can be increased. In some aspects, the total amount of growth of the plant to which the transformed agrobacterium is applied can be increased. The increase in the performance characteristic can be about 1, 2, 3, 4, 5, 6, 7, 8, 9, 10, 11, 12, 13, 14, 15, 16, 17, 18, 19, 20 or more fold as compared to a suitable control. In some aspects, the increase in the performance characteristic can be 3-4 fold more as compared to a suitable control. In some aspects, seed pod increase or seed production can be increased 3-4 fold as compared to a suitable control. In some aspects, height of the plants can be increased about 5, 10, 15, 20, 25, 30, 35, 40, 45, 50, 55, 60, 65, 70, 75, 80, 90, 95, 100 or more percent as compared to a control. In some aspects, height of the plants can be increased about 5 to 100 or more percent as compared to a control.

In some aspects, application of the transformed agrobacterium can result in transient expression of an exogenous gene (e.g. a selectable marker, reporter gene, or any other desired gene) besides any RRV polynucleotide, in one or more cells of the plant to which the transformed agrobacterium is applied. In some aspects, particularly where a reporter gene is fused or operatively linked to the RRV RNA or agRNA in the RRV RNA or RRV agRNA polynucleotide or vector, the plant transiently transformed can have a visual report of disease spread in a plant (e.g. a rose plant) and therefore allow for visual monitoring of infection or RRV resistance.

Also disclosed herein are other transformed cells besides agrobacterium that can be transformed with an RRV RNA, RRV agRNA, or vector described elsewhere herein. In some embodiments, the transformed cell is a plant, bacterial, fungal, or yeast cell. In one embodiment, a plant, bacterial, fungal or yeast cell contains one or more vectors as previously described. Also, within the scope of this disclosure are populations of cells where about 1% to about 100%, or between about 50% and about 75%, or between about 75% and about 100% of the cells within the population contain a vector, a RRV RNA, and/or a RRV agRNA as previously described.

In some embodiments, one or more cells within the population contain more than one type of vector. In some embodiments, all (about 100%) the cells that contain a vector have the same type of vector. In other embodiments, not all the cells that contain a vector have the same type of vector or plurality of vectors. In some embodiments, about 1% to about 100%, or between about 50% and about 75%, or between about 75% and about 100% of the cells within the population contain the same vector or plurality of vectors. In some cell populations, all the cells are from the same species. Other cell populations contain cells from different species. Transfection methods for establishing transformed (transgenic) cells are well known in the art.

Genetically Modified Plants

The infectious RRV polynucleotides and vectors described herein can be used to produce transgenic plants. The present disclosure includes transgenic plants having one or more cells that can contain any of the infectious RRV polynucleotides or vectors described elsewhere herein. The transgenic plant can be a species or cultivar from the genus Rosa, Arabidopsis (e.g. A. thaliana), Nicotiana (e.g. N. benthamiana), Brassica (e.g. Brassica napus), Fragaria, and/or Rubus.

Techniques for transforming a wide variety of plant cells with vectors or naked nucleic acids are well known in the art and described in the technical and scientific literature. See, for example, Weising et al. Ann. Rev. Genet. 1988, 22:421-477. For example, the vector or naked nucleic acid may be introduced directly into the genomic DNA of a plant cell using techniques such as, but not limited to, electroporation and microinjection of plant cell protoplasts, or the recombinant nucleic acid can be introduced directly to plant tissue using ballistic methods, such as DNA particle bombardment.

Microinjection techniques are known in the art and well described in the scientific and patent literature. The introduction of a recombinant nucleic acid using polyethylene glycol precipitation is described in Paszkowski et al. EMBO J. 1984, 3:2717-2722. Electroporation techniques are described in Fromm et al. Proc. Natl. Acad. Sci. USA. 1985, 82:5824. Ballistic transformation techniques are described in Klein et al. Nature. 1987, 327:70-73. The recombinant nucleic acid may also be combined with suitable T-DNA flanking regions and introduced into a conventional Agrobacterium tumefaciens host vector, or other suitable vector. The virulence functions of the Agrobacterium tumefaciens host will direct the insertion of the recombinant nucleic acid including the exogenous nucleic acid and adjacent marker into the plant cell DNA when the cell is infected by the bacteria. Agrobacterium tumefaciens-mediated transformation techniques, including disarming and use of binary vectors, are known to those of skill in the art and are well described in the scientific literature. See, for example, Horsch et al. Science. 1984, 233:496-498; Fraley et al. Proc. Natl. Acad. Sci. USA. 1983, 80:4803; and Gene Transfer to Plants, Potrykus, ed., Springer-Verlag, Berlin, 1995. Other agrobacterium vectors are also described elsewhere herein.

A further method for introduction of the vector or recombinant nucleic acid into a plant cell is by transformation of plant cell protoplasts (stable or transient). Plant protoplasts are enclosed only by a plasma membrane and will therefore more readily take up macromolecules like exogenous DNA. These engineered protoplasts can be capable of regenerating whole plants. Suitable methods for introducing exogenous DNA into plant cell protoplasts include electroporation and polyethylene glycol (PEG) transformation. Following electroporation, transformed cells are identified by growth on appropriate medium containing a selective agent.

The presence and copy number of the exogenous nucleic acid in a transgenic plant can be determined using methods well known in the art, e.g., Southern blotting analysis. Expression of the exogenous root PV phytase nucleic acid or antisense nucleic acid in a transgenic plant may be confirmed by detecting an increase or decrease of mRNA or the root PV phytase polypeptide in the transgenic plant. Methods for detecting and quantifying mRNA or proteins are well known in the art.

Transformed plant cells that are derived by any of the above transformation techniques, or other techniques now known or later developed, can be cultured to regenerate a whole plant. In embodiments, such regeneration techniques may rely on manipulation of certain phytohormones in a tissue culture growth medium, typically relying on a biocide or herbicide selectable marker that has been introduced together with the exogenous nucleic acid. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. Plant Phys. 1987, 38:467-486.

Once the exogenous RRV RNA or agRNA polynucleotide as described elsewhere herein has been confirmed to be stably incorporated in the genome of a transgenic plant, it can be introduced into other plants by sexual crossing. Any of a number of standard breeding techniques can be used, depending upon the species to be crossed.

As discussed with respect to transient expression above, the transgenic plant expressing the infectious RRV clone can increase the performance characteristic or phenotype of the plant to which it is applied. In some aspects, the transgenic plant can have increased growth and/or increased yield (e.g. increased fruit yield, increased flowering, and/or increased seed yield) as compared to a suitable control. In some aspects, the growth rate of the transgenic plant can be increased. In some aspects, the total amount of growth of the transgenic plant can be increased. The increase in the performance characteristic can be about 1, 2, 3, 4, 5, 6, 7, 8, 9, 10, 11, 12, 13, 14, 15, 16, 17, 18, 19, 20 or more -fold. In some aspects, height of the plants can be increased about 5, 10, 15, 20, 25, 30, 35, 40, 45, 50, 55, 60, 65, 70, 75, 80, 90, 95, 100 or more percent as compared to a control. In some aspects, height of the plants can be increased about 5 to 100 or more percent as compared to a control.

In some aspects, the transgenic plant can express an exogenous gene (e.g. a selectable marker, reporter gene, or any other desired gene) besides any RRV polynucleotide, in one or more cells. In some aspects, particularly where a reporter gene is fused or operatively linked to the RRV RNA or agRNA in the RRV RNA or RRV agRNA polynucleotide or vector, the transgenic plant can have a visual report of disease spread in a plant (e.g. rose plant) and therefore allow for visual monitoring of infection or RRV resistance.

A number of embodiments of the invention have been described. Nevertheless, it will be understood that various modifications may be made without departing from the spirit and scope of the invention. Accordingly, other embodiments are within the scope of the following claims.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

Introduction

The most significant technological advance for research into the life cycles of positive and negative strand RNA viruses has been the development of cDNA copies of viral genomes that can be reverse transcribed to produce infectious genomes (Ahlquist P, et al. Proc Natl Acad Sci USA. 1984 81(22):7066-70). This technology permitted genetic manipulation of cDNA for reverse genetic analysis of the virus life cycle and investigations into disease. The second advance in infectious clone technology was the discovery that genetic sequences encoding foreign peptides, large proteins, and small noncoding RNAs can be integrated into specific locations of viral genomes and these recombinant virus clones can be used as tools for reverse genetics of their host, for overexpression of peptides and proteins that can be purified for vaccine production (Dolja V V, et al. Proc Natl Acad Sci USA. 1992 89(21):10208-12; Mardanova E S, et al. BMC Biotechnol. 2015 15:42; Dickmeis C, et al. Biotechnol J. 2014 9(11):1369-79; Tian J, et al. J Exp Bot. 2014 65(1):311-22; Sempere R N, et al. Plant Methods. 2011 7:6; Stevens M, et al. Virus Genes. 2007 34(2):215-21; Rabindran S, et al. Virology. 2001 284(2):182-9; Zhao Y, et al. Arch Virol. 2000 145(11):2285-95; Shivprasad S, et al. Virology. 1999 255(2):312-23). Among plant RNA viruses, incorporation of the green fluorescent protein and its derivates has provided a visual marker of infection that can be used in plant breeding studies to screen germplasm stocks to identify new sources of resistance.

The infectious cDNA technology was rapidly adopted by virologists working on positive strand RNA viruses. Plasmids containing T7, SP6, RNA pol I, RNA pol II promoters were fused to the exact 5′ end of the virus genome to produce transcripts with accurate 5′ ends and transcriptional terminators or hepatitis delta virus ribozymes were located at the 3′ ends to generate transcripts with exact 3′ ends (Jackson A O, et al. Annu Rev Phytopathol. 2016 54:469-98; Bordat A, et al. Virol J. 2015 12:89; Desbiez C, et al. J Virol Methods. 2012 183(1):94-7; Lindbo J A. Plant Physiol. 2007 145(4):1232-40; Boyer J C, et al. Virology. 1994 198(2):415-26). Exact 5′ and 3′ end bases were critical for initiating the first round of translation and replication of transcripts to produce infectious virus genomes. Among negative strand RNA (NSR) viruses, the first infectious clones were produced for viruses with non-segmented genomes belonging to the families Rhabdoviridae, Paramyxoviridae, and Filoviridae (Ebola) [16-20]. Generally, infection is achieved by delivering cDNA copies of viral genomes which produce transcripts that are the anti-genomic RNAs (agRNA). Since agRNA by itself is not infectious, plasmids encoding the nucleocapsid core or subunits of the viral polymerase are co-delivered with the agRNA encoding cDNAs which successfully spurs the replication process. The first infectious clone of a negative strand RNA virus that infects plants was Sonchus yellow net virus (SYNV) (Jackson A O, et al. Annu Rev Phytopathol. 2016 54:469-98; Wang Q, et al. PLoS Pathog. 2015 11(10):e1005223; Jackson A O, et al. Adv Virus Res. 2018 102:23-57; Qian S, et al. Virol J. 2017 14(1):113). The full-length cDNA copy was introduced into a binary vector fused to a duplicated Cauliflower mosaic virus 35S promoter which relies on RNA pol I for transcription. Additional binary plasmids expressing the N (nucleocapsid) protein, P (phosphoprotein) and L (polymerase) protein are co-delivered with the viral cDNA by agroinfiltration to plant leaves. This co-delivery system produces active SYNV infection.

The next major hurdle for NSR viruses was to produce infectious clones of viruses with multiple genome segments. In 1999 the first infectious cDNA for Influenza virus was prepared (Neumann G, et al. Proc Natl Acad Sci USA. 1999 96(16):9345-50; Neumann G, et al. Adv Virus Res. 1999 53:265-300). The eight genome segments were produced using a promoter that depended up on the cellular RNA pol I for synthesis of agRNA alongside four plasmids that expressed proteins required for viral replication and transcription (PB1, PB2, PA, NP).

This Example can demonstrate the generation and use of an infectious clone of Rose rosette virus (RRV). RRV is a member of the Emaravirus genus and has seven genome segments (Mielke-Ehret N, et al. Viruses. 2012 4(9):1515-36). Each cDNA was synthesized de novo fused to the duplicated CaMV 35S promoter to produce an exact 5′ end and a 3′ hepatitis delta virus ribozyme (HDR) to produce an exact 3′ end. This Example can demonstrate, inter alia, successful introduction of a green fluorescent protein (GFP) reporter protein, which was fused to the movement protein in RNA3 and the putative envelope glycoprotein (G) encoded by RNA 2. The RNA2 fusion construct can allow for visual identification, evaluation, and monitoring of glycoprotein incorporation into virions. This Example can also demonstrate the introduction of an iLOV fluorescent protein into RNAS as a gene replacement.

Roses are the economically most important ornamental plants belonging to the family Rosaceae and comprise 30% of the floriculture industry. Rose rosette virus has been devastating roses and the rose industry in the USA, causing millions of dollars in losses. Typical symptoms of RRV are described as rapid stem elongation, followed by breaking of axillary buds, leaflet deformation and wrinkling, bright red pigmentation, phyllody, and increased thorniness. This enhanced visual reporter system that can be demonstrated by this Example can be used forr screening rose germplasm stocks to identify new sources of resistance.

Materials and Methods

Plant Materials and Virus Inoculation.

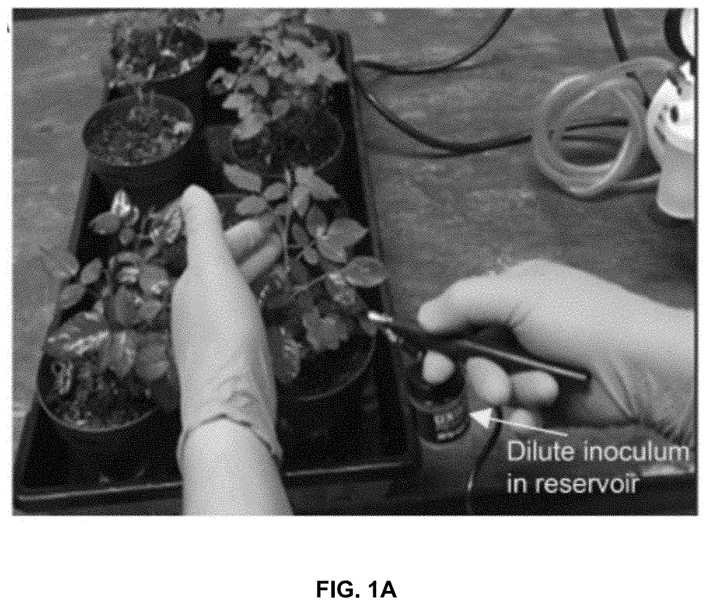

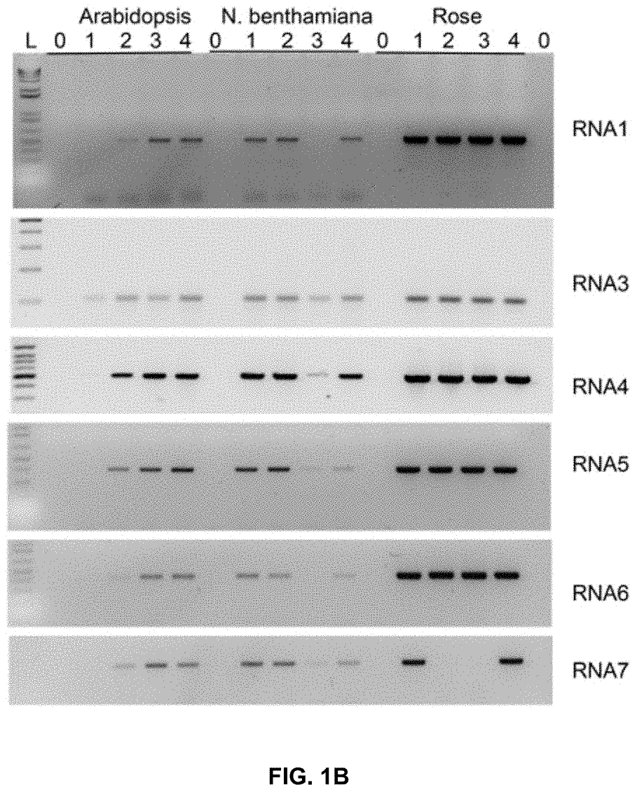

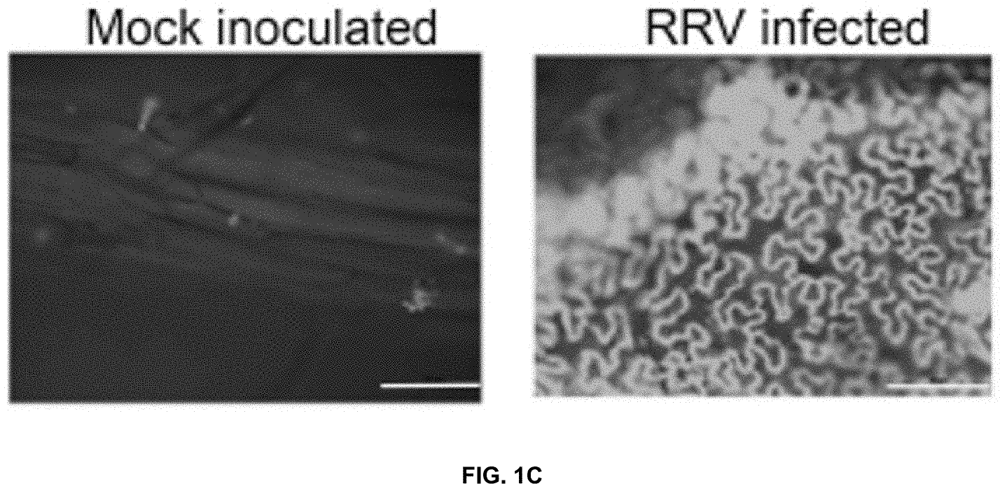

Arabidopsis thaliana plants were grown at 23° C. with 10 h/14 h (day/night) photoperiod in a growth chamber. Nicotiana benthamiana and rose plants were grown at 23° C. with 16 h/8 h (day/night) photoperiod in a growth chamber. Four-week-old plants (Arabidopsis thaliana and Nicotiana benthamian) were inoculated with sap prepared from virus infected rose plants. Virus-infected rose (cv Julia Child) leaves (0.5 g) were ground in 20 mL (1:30 w/v) in 0.05 M phosphate buffer (pH 7.0) supplemented with 1 unit of RNase inhibitor. Sap was loaded to reservoir of an artist airbrush. Plants were lightly dusted with carborundum and sap was applied using the high-pressure artist air brush (FIG. 1A).

Four-week-old plants (Arabidopsis thaliana and Nicotiana benthamiana) were also inoculated with RRV infectious clone. Agrobacterium (GV3101) cultures harboring pCB301 derivative constructs for each RRV agRNA segment were grown overnight in YEP media and then resuspended in MES buffer (10 mM MgCl₂, 10 mM MES, pH 5.6, and 150 uM acetosyringone) and adjusted to an optical density A₆₀₀ of 1.0. Cultures were incubated for 2-4 hours and equal volumes of each Agrobacterium culture for RRV agRNA segment were mixed at 1.0 OD. Mixed cultures were loaded to a 1 ml syringe for infiltration of N. benthamiana, Arabidopsis, or rose plants.

Plasmid Construction.

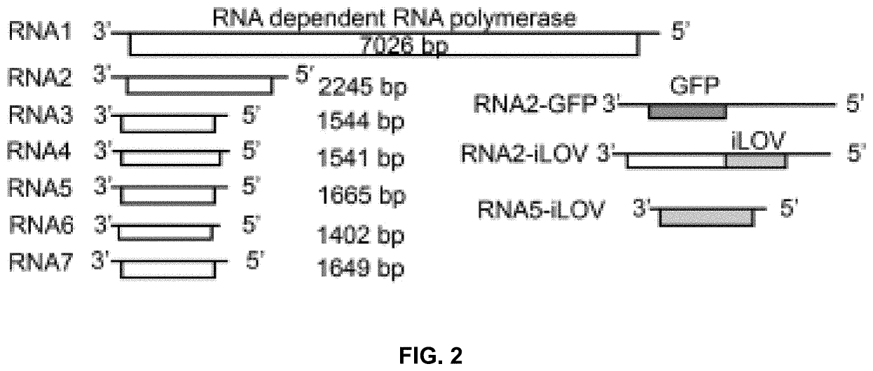

FIG. 2 shows a diagrammatic representation of antigenomic RRV constructs. The lines represent the 3′ to 5′ orientation of the genome segments. The open boxes indicate the open reading frames encoded by each segment. The size in base pairs for each segment is provided. The modifications are where GFP or iLOV were inserted into the genome are also identified. The full length antigenomic (ag)RNA sequences for RRV segments 1 through 4 (Laney A G, et al. J Gen Virol. 2011 92(Pt 7):1727-32) were synthesized (pUC57) and cloned into pCB301-HDV plasmids by GenScript (Piscataway, N.J.). The pCB301-HDV plasmid is a binary plasmid with a duplicated Cauliflower mosaic virus (CaMV) 35S promoter and 3′ Hepatitis delta virus ribozyme (HDRz) sequence. The cDNAs encoding agRNAs for RRV segments 5, 6 and 7 (Di Bello P L, et al. Virus Res. 2015 210:241-4) were amplified using Platinum SuperFi PCR Master Mix and primers with 15 nt adapters that overlap pCB301 sequences. The high fidelity directional In-Fusion® HD Cloning Kit (Takara Bio USA, Inc.) was used to introduce each amplified cDNAs into the pCB301-HDV vector to produce exact sequence fusion with the CaMV 35S promoter and HDRz.

The amino acid sequence for pCB301 plasmid is:

(SEQ ID NO: 1)
MAKMRISPELKKLIEKYRCVKDTEGMSPAKVYKLVGENENLYLKMTDS
RYKGTTYDVEREKDMMLWLEGKLPVPKVLHFERHDGWSNLLMSEADGV
LCSEEYEDEQSPEKIIELYAECIRLFHSIDISDCPYTNSLDSRLAELD
YLLNNDLADVDCENWEEDTPFKDPRELYDFLKTEKPEEELVFSHGDLG
DSNIFVKDGKVSGFIDLGRSGRADKVVYDIAFCVRSIREDIGEEQYVE
LFFDLLGIKPDWEKIKYYILLDELF.

The nucleic acid sequence for pCB301 plasmid is:

(SEQ ID NO: 2)
aagcttgcat gcctgcagtc aacatggtgg agcacgacac tctcgtctac tccaagaata
tcaaagatac agtctcagaa gaccagaggg ctattgagac ttttcaacaa agggtaatat
cgggaaacct cctcggattc cattgcccag ctatctgtca cttcatcgaa aggacagtag
aaaaggaaga tggcttctac aaatgccatc attgcgataa aggaaaggct atcgttcaaa
gaatgcctct accgacagtg gtcccaaaga tggacccccc acccacgagg aacatcgtgg
aaaaagaaga cgttccaacc acgtcttcaa agcaagtgga ttgatgtgat aacatggtgg
agcacgacac tctcgtctac tccaagaata tcaaagatac agtctcagaa gaccagaggg
ctattgagac tttcaacaaa gggtaatatc gggaaacctc ctcggattcc attgcccagc
tatctgtcac ttcatcgaaa ggacagtaga aaaggaagat ggcttctaca aatgccatca
ttgcgataaa ggaaaggcta tcgttcaaga atgcctctac cgacagtggt cccaaagatg
gacccccacc cacgaggaac atcgtggaaa aagaagacgt tccaaccacg tcttcaaagc
aagtggattg atgtgatatc tccactgacg taagggatga cgcacaatcc cactatcctt
cgcaagaccc ttcctctata taaggaagtt catttcattt ggagaggcct gacctgcagg
tcgactctag aggatccccg ggtcggcatg gcatctccac ctcctcgcgg tccgacctgg
gcatccgaag gaggacgtcg tccactcgga tggctaaggg agagctcgaa tttccccgat
cgttcaaaca tttggcaata aagtttctta agattgaatc ctgttgccgg tcttgcgatg
attatcatat aatttctgtt gaattacgtt aagcatgtaa taattaacat gtaatgcatg
acgttattta tgagatgggt ttttatgatt agagtcccgc aattatacat ttaatacgcg
atagaaaaca aaatatagcg cgcaaactag gataaattat cgcgcgcggt gtcatctatg
ttactagatc ggaattcaga ttgtcgtttc ccgccttcag tttaaactat cagtgtttga
caggatatat tggcgggtaa acctaagaga aaagagcgtt tattagaata atcggatatt
taaaagggcg tgaaaaggtt tatccgttcg tccatttgta tgtgcatgcc aaccacagga
gatctcagta aagcgctggc tgaaccccca gccggaactg accccacaag gccctagcgt
ttgcaatgca ccaggtcatc attgacccag gcgtgttcca ccaggccgct gcctcgcaac
tcttcgcagg cttcgccgac ctgctcgcgc cacttcttca cgcgggtgga atccgatccg
cacatgaggc ggaaggtttc cagcttgagc gggtacggct cccggtgcga gctgaaatag
tcgaacatcc gtcgggccgt cggcgacagc ttgcggtact tctcccatat gaatttcgtg
tagtggtcgc cagcaaacag cacgacgatt tcctcgtcga tcaggacctg gcaacgggac
gttttcttgc cacggtccag gacgcggaag cggtgcagca gcgacaccga ttccaggtgc
ccaacgcggt cggacgtgaa gcccatcgcc gtcgcctgta ggcgcgacag gcattcctcg
gccttcgtgt aataccggcc attgatcgac cagcccaggt cctggcaaag ctcgtagaac
gtgaaggtga tcggctcgcc gataggggtg cgcttcgcgt actccaacac ctgctgccac
accagttcgt catcgtcggc ccgcagctcg acgccggtgt aggtgatctt cacgtccttg
ttgacgtgga aaatgacctt gttttgcagc gcctcgcgcg ggattttctt gttgcgcgtg
gtgaacaggg cagagcgggc cgtgtcgttt ggcatcgctc gcatcgtgtc cggccacggc
gcaatatcga acaaggaaag ctgcatttcc ttgatctgct gcttcgtgtg tttcagcaac
gcggcctgct tggcctcgct gacctgtttt gccaggtcct cgccggcggt ttttcgcttc
ttggtcgtca tagttcctcg cgtgtcgatg gtcatcgact tcgccaaacc tgccgcctcc
tgttcgagac gacgcgaacg ctccacggcg gccgatggcg cgggcagggc agggggagcc
agttgcacgc tgtcgcgctc gatcttggcc gtagcttgct ggaccatcga gccgacggac
tggaaggttt cgcggggcgc acgcatgacg gtgcggcttg cgatggtttc ggcatcctcg
gcggaaaacc ccgcgtcgat cagttcttgc ctgtatgcct tccggtcaaa cgtccgattc
attcaccctc cttgcgggat tgccccgact cacgccgggg caatgtgccc ttattcctga
tttgacccgc ctggtgcctt ggtgtccaga taatccacct tatcggcaat gaagtcggtc
ccgtagaccg tctggccgtc cttctcgtac ttggtattcc gaatcttgcc ctgcacgaat
accagcgacc ccttgcccaa atacttgccg tgggcctcgg cctgagagcc aaaacacttg
atgcggaaga agtcggtgcg ctcctgcttg tcgccggcat cgttgcgcca catctaggta
ctaaaacaat tcatccagta aaatataata ttttattttc tcccaatcag gcttgatccc
cagtaagtca aaaaatagct cgacatactg ttcttccccg atatcctccc tgatcgaccg
gacgcagaag gcaatgtcat accacttgtc cgccctgccg cttctcccaa gatcaataaa
gccacttact ttgccatctt tcacaaagat gttgctgtct cccaggtcgc cgtgggaaaa
gacaagttcc tcttcgggct tttccgtctt taaaaaatca tacagctcgc gcggatcttt
aaatggagtg tcttcttccc agttttcgca atccacatcg gccagatcgt tattcagtaa
gtaatccaat tcggctaagc ggctgtctaa gctattcgta tagggacaat ccgatatgtc
gatggagtga aagagcctga tgcactccgc atacagctcg ataatctttt cagggctttg
ttcatcttca tactcttccg agcaaaggac gccatcggcc tcactcatga gcagattgct
ccagccatca tgccgttcaa agtgcaggac ctttggaaca ggcagctttc cttccagcca
tagcatcatg tccttttccc gttccacatc ataggtggtc cctttatacc ggctgtccgt
catttttaaa tataggtttt cattttctcc caccagctta tataccttag caggagacat
tccttccgta tcttttacgc agcggtattt ttcgatcagt tttttcaatt ccggtgatat
tctcatttta gccatttatt atttccttcc tcttttctac agtatttaaa gataccccaa
gaagctaatt ataacaagac gaactccaat tcactgttcc ttgcattcta aaaccttaaa
taccagaaaa cagctttttc aaagttgttt tcaaagttgg cgtataacat agtatcgacg
gagccgattt tgaaaccaca attatgggtg atgctgccaa ctcgagagcg ggccgggagg
gttcgagaag ggggggcacc ccccttcggc gtgcgcggtc acgcgcacag ggcgcagccc
tggttaaaaa caaggtttat aaatattggt ttaaaagcag gttaaaagac aggttagcgg
tggccgaaaa acgggcggaa acccttgcaa atgctggatt ttctgcctgt ggacagcccc
tcaaatgtca ataggtgcgc ccctcatctg tcagcactct gcccctcaag tgtcaaggat
cgcgcccctc atctgtcagt agtcgcgccc ctcaagtgtc aataccgcag ggcacttatc
cccaggcttg tccacatcat ctgtgggaaa ctcgcgtaaa atcaggcgtt ttcgccgatt
tgcgaggctg gccagctcca cgtcgccggc cgaaatcgag cctgcccctc atctgtcaac
gccgcgccgg gtgagtcggc ccctcaagtg tcaacgtccg cccctcatct gtcagtgagg
gccaagtttt ccgcgaggta tccacaacgc cggcggccgc ggtgtctcgc acacggcttc
gacggcgttt ctggcgcgtt tgcagggcca tagacggccg ccagcccagc ggcgagggca
accagcccgg tgagcgtcta gtggactgat gggctgcctg tatcgagtgg tgattttgtg
ccgagctgcc ggtcggggag ctgttggctg gctggtggca ggatatattg tggtgtaaac
aaattgacgc ttagacaact taataacaca ttgcggacgt ttttaatgta ctggggtggt
ttt

TABLE 1
Primers for cloning and RACE

| Primer Pairs | Primer sequences (5′ to 3′) |
|---|---|
| Attb_RRV_F | GGGGACAAGTTTGTACAAAAAAGCAGGCTTAAGTAGTGAA |
| | CTCC (SEQ ID NO: 21) |
| Attb_RRV_R | GGGGACCACTTTGTACAAGAAAGCTGGGTTAGTAGTGTTC |
| | TCC (SEQ ID NO: 22) |

Sequence in bold overlap Gateway Attb sequence. RRV 5′ and 3′ end primers
overlap conserved terminal sequences to amplify full-length segments 5-7 from
infected plants

| | |
|---|---|
| IF_agR5_F | TTTCATTTGGAGAGGAGTAGTGAACTCCCACAATAATTTCG |
| | ATTAACA (SEQ ID NO: 23) |
| IF_agR5_R | ATGCCATGCCGACCCAGTAGTGTTCTCCCACAAAAATATCA |
| | AATTCAATG (SEQ ID NO: 24) |
| IF_agR6_F | TTTCATTTGGAGAGGAGTAGTGAACTCCCTATAAAACCAAG |
| | CG (SEQ ID NO: 25) |
| IF_agR6_R | ATGCCATGCCGACCCAGTAGTGTTCTCCCTATAAACTTCAG |
| | CAG (SEQ ID NO: 26) |
| IF_agR7_F | TTCATTTGGAGAGGAGTAGTGAACTCCCACAATTAAATAGA |
| | TTTTGAAAAAAAG (SEQ ID NO: 27) |

IF_agR7_R

ATGCCATGCCGACCCAGTAGTGTTCTCCCACAAATTAATCA
AAAAACTG (SEQ ID NO: 28)

Sequences in bold overlap sequences for In-Fusion® cloning into pCB301. RRV 5′
and 3′ end primers overlap conserved terminal sequences of agRNA segments.

| | |
|---|---|
| R5_iLOV_F | AAGCTTGCACCAGTGATGGCTAGCATAGAGAAGAATTTCG |
| | TCA (SEQ ID NO: 29) |
| R5_iLOV_R | AAATATATTGTAACTCTATACATGATCACTTCCATCGAGCT |
| | G (SEQ ID NO: 30) |

Sequences in bold overlap sequences for In-Fusion® cloning to replace ORF5

| | |
|---|---|
| pCB301_R5.F | AGTTACAATATATTTGTTCCAATGATGGCAATATATTTCAT |
| | (SEQ ID NO: 31) |
| pCB301_R5.R | CACTGGTGCAAGCTTTAAAAGAGTTT (SEQ ID NO: 32) |

In-fusion clone P5-iLOV

| | |
|---|---|
| NbPDS_Xbal_F | ATCGTCTAGACTGTGATAAATGTCCATATATGGTTTGACAG |
| | (SEQ ID NO: 33) |
| NbPDS_Xbal_R | ATCGTCTAGAGGGTTTTGACAACATGATACTTCAATATTTTT |
| | G (SEQ ID NO: 34) |

Cloning fragment of N. benthamiana phytoene desaturase. Engineered Xbal
restriction site in bold

| | |
|---|---|
| RmPDS_Xbal__ | ATCGTCTAGAATTTCTTCAGGAGAAACACGGTTC (SEQ ID |
| F | NO: 35) |
| RmPDS_Xbal_ | ATCGTCTAGACCAACTAGTTTGTCCAATTTCTTGAAAT (SEQ |
| R | ID NO: 36) |

Cloning fragment of R. multiflora phytoene desaturase. Engineered Xbal restriction
sites in bold

| | |
|---|---|
| smeGFP_F | ATCGAGGCCTATGGTTTCTAAGGGTGAGGA (SEQ ID |
| | NO: 37) |
| smeGFP_R | CGATCCCGGGTCACTTGTAAAGCTCA (SEQ ID NO: 38) |

Clone G protein fused GFP. Engineered Stul, Smal restriction sites in bold

| | |
|---|---|
| 2AsmeGFP_F | ATCGAGGCCTCAGCTTCTGAACTTTGATCTG (SEQ ID |
| | NO: 39) |
| 2AsmeGFP_R | CGATCCCGGGTCACTTGTAAAGCTCA (SEQ ID NO: 40) |

Clone P4-2A GFP. Engineered Stul, Smal restriction sites in bold

| | | |
|---|---|---|
| R1-5RACE-R | GATTACGCCAAGCTTAGTTGGCATTTGATGTAAG | 1-708bp |
| | ACTCAGGAC (SEQ ID NO: 41) | |
| NR1-5RACE-R | GATTACGCCAAGCTTTTGTCACTGAAAGAATCAA | 1-492bp |
| | CCCACAGA (SEQ ID NO: 42) | |
| R1-3RACE-F | GATTACGCCAAGCTTCATCCATTATTGTGGGCCA | 6133- |
| | GTGTTTACC (SEQ ID NO: 43) | 7026bp |
| NR1-3RACE-F | GATTACGCCAAGCTTCCTGTTTCTATGAAGCCAG | 6390- |
| | ATGGTGAG (SEQ ID NO: 44) | 7026bp |
| R2-5RACE-R | GATTACGCCAAGCTTATATTAGTCCATTGCACTG | 1-948bp |
| | ACCCTGGT (SEQ ID NO: 45) | |
| NR2-5RACE-R | GATTACGCCAAGCTTGTGGCACATTCAGATTAGA | 1-568bp |
| | GCATAGTAAG (SEQ ID NO: 46) | |
| R2-3RACE-F | GATTACGCCAAGCTTATCTGCTAAGCATTCAACG | 1377- |
| | GCTCCA (SEQ ID NO: 47) | 2245bp |
| NR2-3RACE-F | GATTACGCCAAGCTTTGGAGTTGGACAATCTAGA | 1691- |
| | TGGGAGAAC (SEQ ID NO: 48) | 2245bp |
| R2-3RACE-F | GATTACGCCAAGCTTATCTGCTAAGCATTCAACG | 1-822bp |
| | GCTCCAT (SEQ ID NO: 49) | |
| NR2-3RACE-F | GATTACGCCAAGCTTTGGAGTTGGACAATCTAGA | 1-687bp |
| | TGGGAGAAC (SEQ ID NO: 50) | |
| R3-5RACE-R | GATTACGCCAAGCTTTGAGCTCTATCCAGCTGAA | 869- |
| | GTGTTGGCT (SEQ ID NO: 51) | 1544bp |
| NR3-5RACE-R | GATTACGCCAAGCTTGCTAGAGACTACTCCTTTC | 1142- |
| | GATGATGCT (SEQ ID NO: 52) | 1544bp |
| R3-3RACE-F | GATTACGCCAAGCTTGAGTCCATTCCCATTTGTC | 1-653bp |
| | CTGCCAG (SEQ ID NO: 53) | |
| NR3-3RACE-F | GATTACGCCAAGCTTTCCAGGGACCTAGAAAGAT | 1-422bp |
| | AAGAAACAGC (SEQ ID NO: 54) | |
| R4-5RACE-R | GATTACGCCAAGCTTGTCTTAACAAGGTTAGGGC | 833- |
| | CAAGCTCAA (SEQ ID NO: 55) | 1541bp |
| NR4-5RACE-R | GATTACGCCAAGCTTCCCTCAAGAGATGGACTAC | 1050-1541bp |
| | GGCTACATT (SEQ ID NO: 56) | |
| R4-3RACE-F | GATTACGCCAAGCTTGAATCCAGCTGTAGATTGA | 1-708bp |
| | GTTTCACCC (SEQ ID NO: 57) | |
| NR4-3RACE-F | GATTACGCCAAGCTTCAAGATTAAGCTTCCCCTG | 1-492bp |
| | AGCCAACA (SEQ ID NO: 58) | |

Unique StuI and SmaI restriction sites were engineered into the 3′ end of ORF4 in pCB301-RNA4. GFP was PCR amplified using primers containing StuI and SmaI restriction sites. Linearized vector and digested PCR products were ligated and used to transform DH5 alpha cells. GFP was inserted into this location as an in-frame fusion with the ORF4 protein (putative movement protein). A second version included the sequence encoding the 2a peptide of Foot and Mouth Disease Virus (FMDV) (Röder J, et al. Front Plant Sci. 2017 8:1125) between the ORF4 and GFP coding sequences. Upon translation the 2a peptide autocatalytically cleaves the fusion producing mature GFP. Plasmids were maintained in Escherichia coli DH5alpha cells. The pCB301 based derivative plasmids were also maintained in Agrobacterium tumefaciens strain GV3101.

RT-PCR and dsRNA Binding-Dependent Fluorescence complementation Assay (dRBFC).

Total RNAs were extracted from the upper leaves of RRV-infected and mock treated plants with Qiagen Plant RNAeasy® Isolation kit. RT-PCR was carried out using reverse transcriptase and high-fidelity DNA polymerase with RRV specific primers (Table 2). PCR products were separated in 1.0% agarose gels. PCR products were also sequenced to confirm the RRV sequences were stably maintained.

dsRBFC was carried out for fluorescence labelling RRV dsRNA replication intermediates according to Cheng et al (2015) (Cheng X, et al. Virology. 2015 485:439-51). Dr Aiming Wang (Southern Crop & Food Research Center, Agriculture and Agri-Food Canada) provided agrobacteria containing the flock house virus (FHV) B2-YN and BY-YC constructs. These binary constructs contain the coding sequence or the dsRNA binding domain of the FHV B2 protein fused to the N-terminal or C-terminal fragment of YFP. Agrobacteria expressing B2-YN and B2-YC were mixed in equal ratin and directly infiltrated into N. benthamiana leaves that were inoculated with RRV containing sap and control leaves that were treated with buffer only. The YFP fluorescence was visualized using a Nikon Eclipse 90i epifluorescence microscope.

Immunoblot Detection of Plant Viruses.

Total proteins were extracted from healthy and infected leaves and evaluated by immunoblotting. Proteins were separated by SDS-PAGE and then transferred to PVDF membranes and probed with GFP antisera. Membranes were also stained with Ponceau S.

Results

Mechanical Inoculation of Arabidopsis, Nicotiana Benthamiana, and Roses Using RRV Containing Plant Sap.

RRV is a negative strand RNA virus with seven genome segments and is typically transmitted by erythroid mites to rose plants. Most researchers rely on viruliferous mites to deliver virus to plants as the preferred method for inoculation and mechanical delivery of RRV to test plants has not been routinely demonstrated. In this study, homogenate inoculum was prepared by grinding infected rose tissue in 0.5 M phosphate buffer (pH 7.0). Sap was applied to Arabidopsis, Nicotiana benthamiana and roses (“Old Blush” variety) using a pressurized artist airbrush (FIG. 1A).

Negative strand RNA viruses produce antigenomic (ag) RNAs generated by the viral RNA dependent RNA polymerase (RdRp). Double strand (ds) RNAs accumulate as replication intermediates. After 6 days, two assays were carried out to detect the production of agRNAs and dsRNAs in virus inoculated leaves, as evidence that the sap inoculations resulted in productive infection. First, RT-PCR was carried out. RNA was extracted and cDNA was prepared using primers that hybridized to antigenomic (ag) RNAs. Diagnostic RT-PCRs produced the expected size fragments between 104 and 500 nt (Table 2, FIG. 1B) confirmed accumulation of agRNAs representing the seven segments in rose, A. thaliana and N. benthamiana leaves.

TABLE 2
Infection Characteristics in Arabidopsis

| | pCB301 mock | RRV seg 1-4 | RRV seg 1-7 |
|---|---|---|---|
| Inflorescence | 59 | 45 | 45 |
| emergence (days) | | | |
| Plants with lateral | 2(4) | 6(6) | 6(6) |
| inflorescence branches | | | |
| Plants with secondary | 2(4) | 6(6) | 6(6) |
| inflorescence branches | | | |
| Plants with tertiary | 0(4) | 6(6) | 6(6) |
| inflorescence branches | | | |
| Plants with Aerial | 3(4) have <10 | 6(6) have >30 | 6(6) have >40 |
| Rosettes | aerial rosettes | aerial rosettes | aerial rosettes |
| Average Height | 22.0 cm | 51.0 cm | 45.0 cm |
| Average Number of | 50 (short) | 250 (long) | 200 (long) |
| Siliques | | | |
| Seed germination | 100% | 100% | 100% |

Second, the dsRBFC assay which detects dsRNA in living N. benthamiana leaves was used to detect RRV dsRNAs. The dsRBFC consists of two FHV B2 dsRNA binding domains fused to N- and C-terminal halves of YFP. Binding by the fusion proteins to common dsRNAs brings the two halves of YFP together and produces visible yellow fluorescence (Cheng X, et al. Virology. 2015 485:439-51). Two agrobacteria cultures containing B2-YN and B2-YC were mixed and infiltrated into RRV inoculated and mock-inoculated N. benthamiana leaves. Leaf segments were examined using epifluorescence microscopy. YFP fluorescence was seen throughout the epidermal cells of RRV infected leaves but was not reconstituted in mock-inoculated leaves (FIG. 10). The combined results of RT-PCR and dsRBFC confirm that RRV can successfully infect rose, A. thaliana, and N. benthamiana following mechanical inoculation.

Construction of functional infectious clone of RRV for experimental studies.

Synthetic cDNAs for agRNA1, agRNA2, agRNA3 and agRNA4 were synthesized do novo and inserted into the small binary plasmid pCB301-HDV, which contains the CaMV 35S promoter, HDV antigenomic ribozyme, and Nos terminator. The antigenomic cDNA positioned next to the CaMV 35S promoter and HDRz to produce viral transcripts with authentic 5′ and 3′ ends (FIG. 1B). Then cDNAs encoding the agRNA5, agRNA6, and agRNA7 were directly amplified using total RNA isolated from infected roses, and then introduced into the pCB301-HDV backbone. All constructs were confirmed by restriction digestion and sequencing. Plasmids were mobilized into A. tumefaciens and bacteria harboring each plasmid were mixed in equal ratio for subsequent experiments.

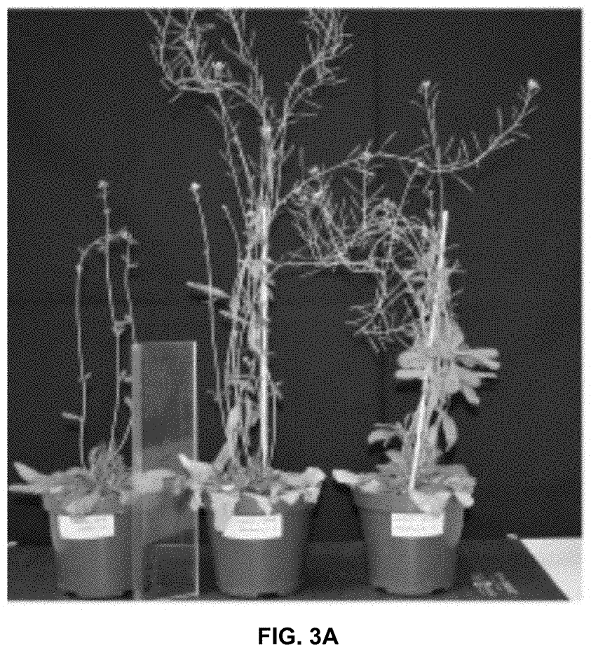

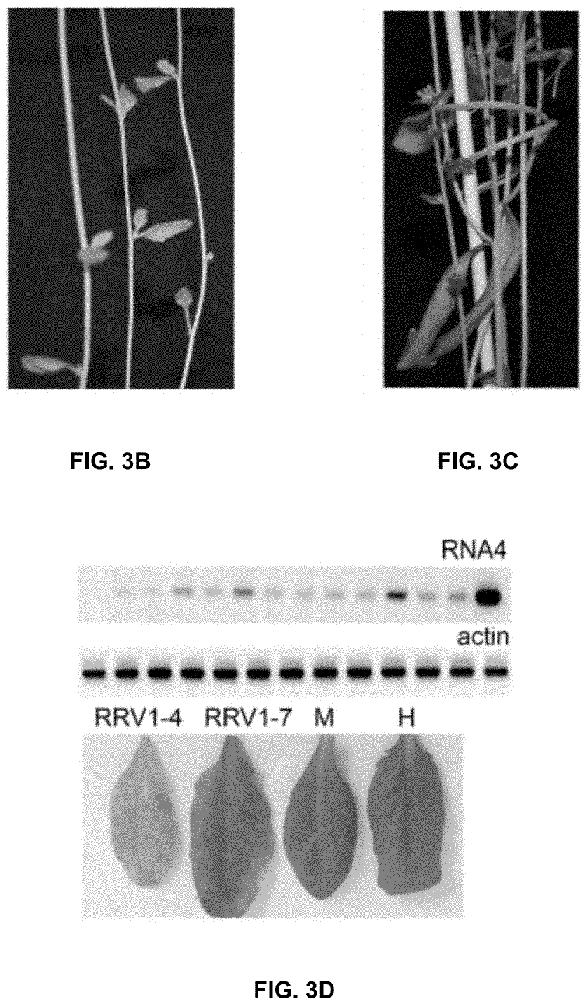

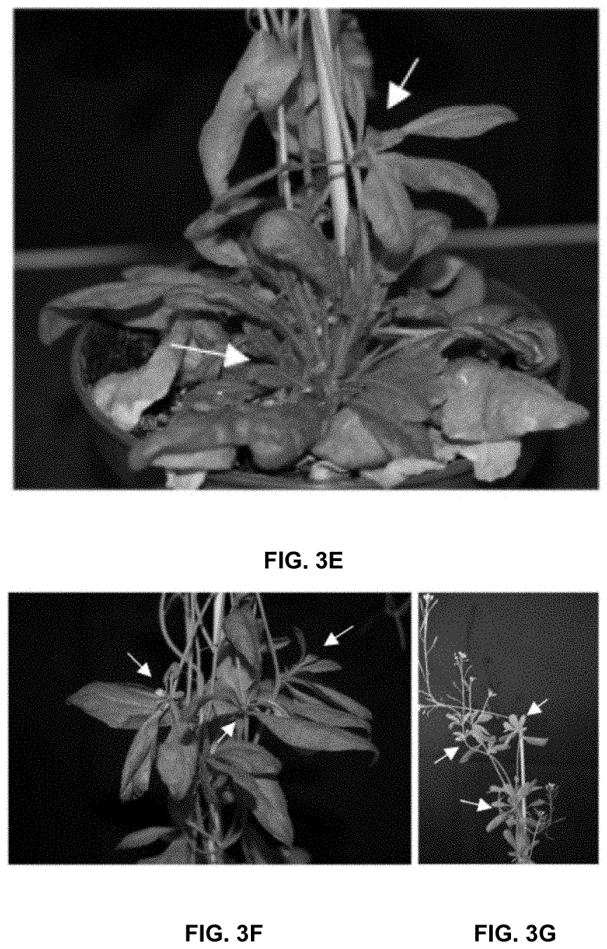

A. thaliana (Col-0) leaves were inoculated by agro-infiltration to deliver the combination of RRV segments 1, 2, 3 and 4 (RRV1-4) and another set was inoculated by agro-infiltration to deliver all the combination of RRV segments 1 through 7 (RRV1-7). Four to six plants were inoculated with each experiment and experiments were repeated multiple times. Plants were grown in short day length (10 h light and 14 h dark) and observed for 60 days. Plant height from the soil surface to the top of the inflorescences were measured and the average height for mock treated A. thaliana plants was 22.0 cm. Plants that are infected with RRV1-4 or RRV1-7 were taller than mock treated plants, ranging in height from 45-51 cm (FIG. 3A, Table 3). The inoculated leaves primarily displayed symptoms that were mild yellow mottling which was not seen on the mock treated and untreated leaves (FIGS. 3B and 3C).

The plant body plan was significantly altered in virus infected plants. Plants infected with RRV1-7 showed more basal leaves in the vegetative rosette than mock-inoculated plants and RRV1-4 infected plants. Bolting occurred around 59 days after treatment in mock inoculated plants and at 45 days in RRV1-4 and RRV1-7 infected plants. After bolting, mock inoculated plants produced three inflorescence stems with five to six cauline leaves and a solitary flower (FIG. 3A). All plants infected with RRV1-4 or RRV 1-7 produced the three major inflorescences with multiple leaves and higher order branches with a greater abundance of flowers. RRV1-7 infected plants showed aerial rosettes that form at the axils where cauline leaves normally develop, suggesting that virus infection alters the developmental patterning of axillary meristems (Table 3, FIGS. 3E-3G). The number of siliques on mature plants at 45 d was 4-5 fold greater than mock treated plants (Table 3). Seeds were collected from plants, germinated on media, and 100% germinated producing healthy plants (Table 3). FIG. 3D shows the PCR gels confirm the plants are infected using primers that amplify RNA 4 sequences.

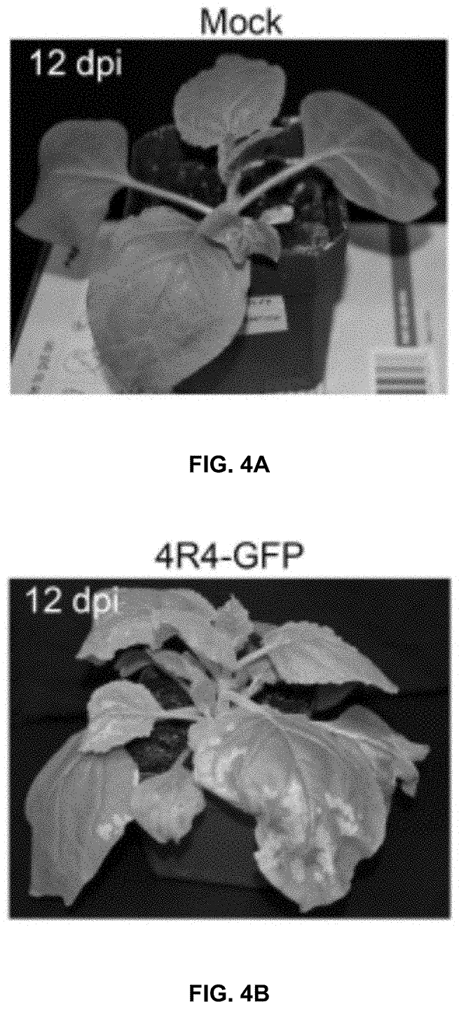

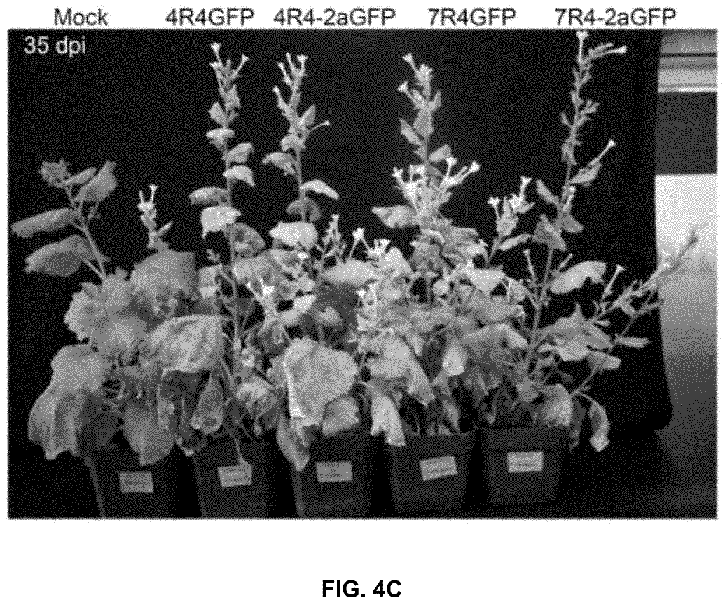

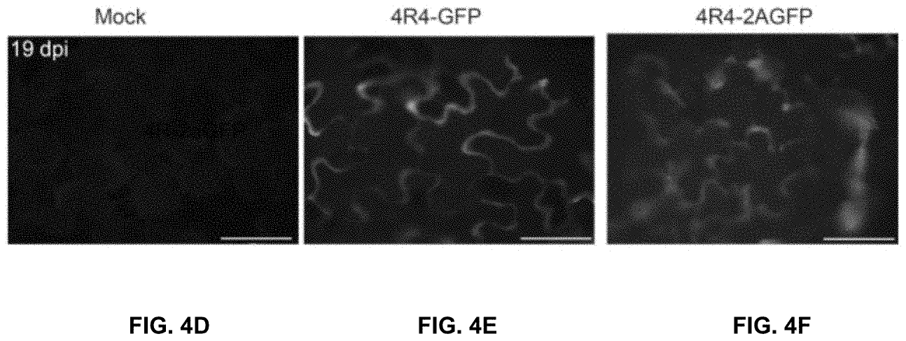

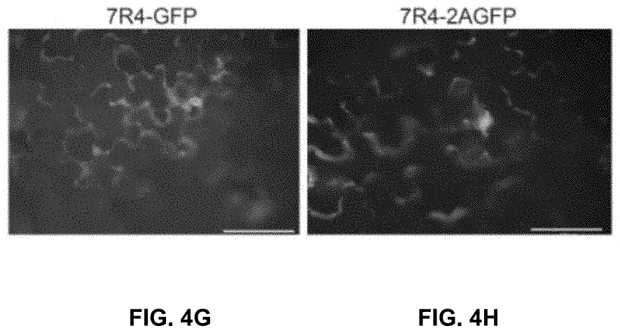

FIGS. 4A-4H shows healthy and virus infected plants at 12 and 35-days. Infected N. benthamiana plants show necrosis, but also more flowers than the healthy control. FIGS. 4D-H shows florescent micrographs showing GFP in infected leaves.

TABLE 3
Total proportion of Plants systemically infected
following agroinfiltration with constructs

| Constructs | Arabidopsis | N. benthamiana | Roses |
|---|---|---|---|
| pCB301 | 0/12 | 0/12 | 0/4 |
| RRV1-4 | 20/20 | 8/12 | |
| RRV1-7 | 18/18 | 10/12 | |
| RRV1-4GFP | 12/12 | 12/12 | |
| RRV1-7GFP | 12/12 | 12/12 | |
| Sap inoculated | 12/12 | 12/12 | 4/4 |
| plants | | | |
| Buffer treated | 0/8 | 0/8 | 0/4 |

The proportion of infected Arabidopsis plants were pooled from three experiments and the proportion of infected N. benthamiana were pooled from two experiments.

### Example 2

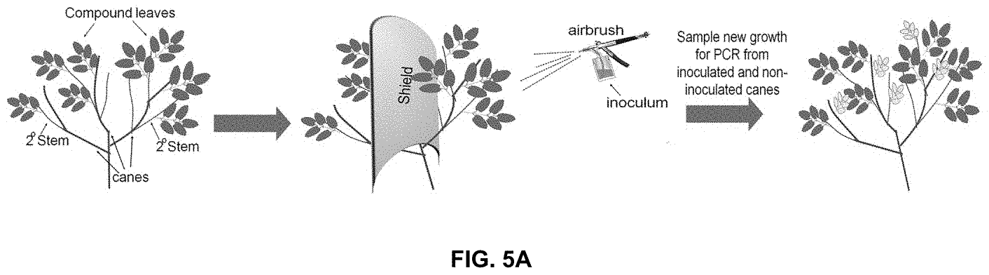

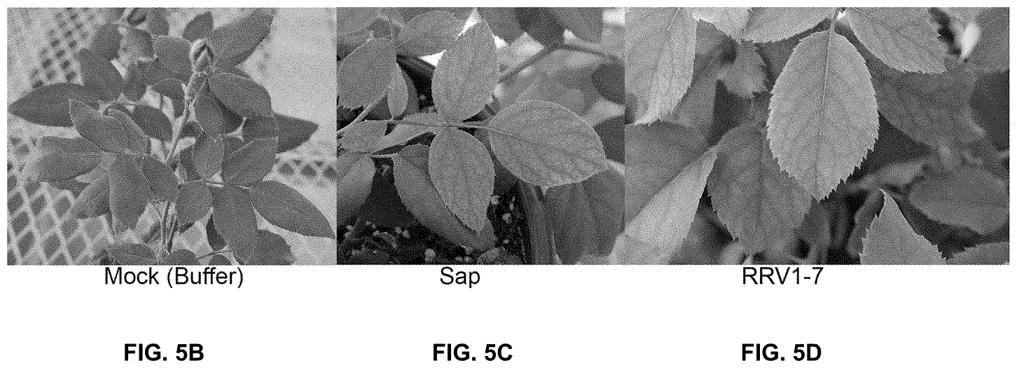

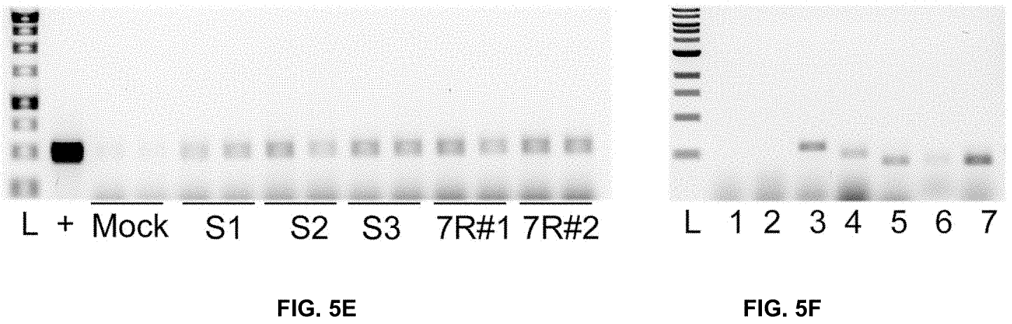

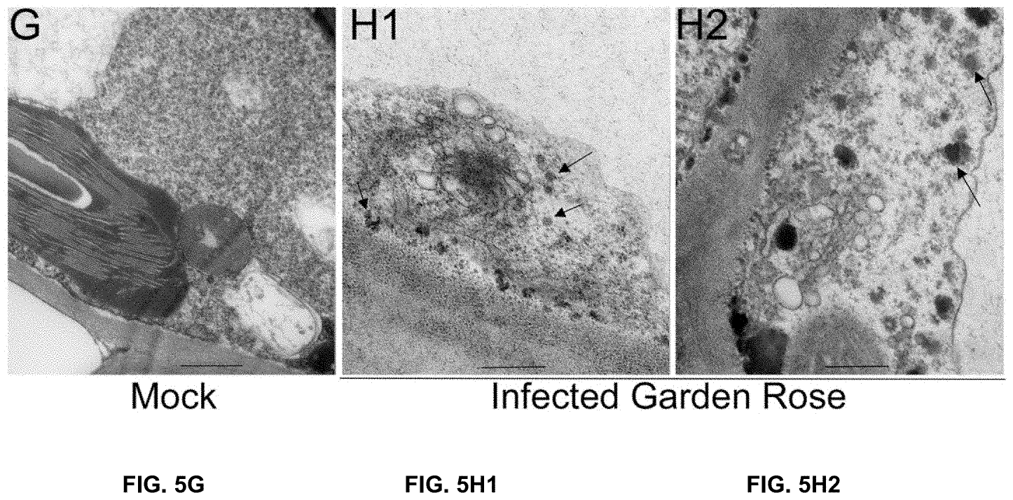

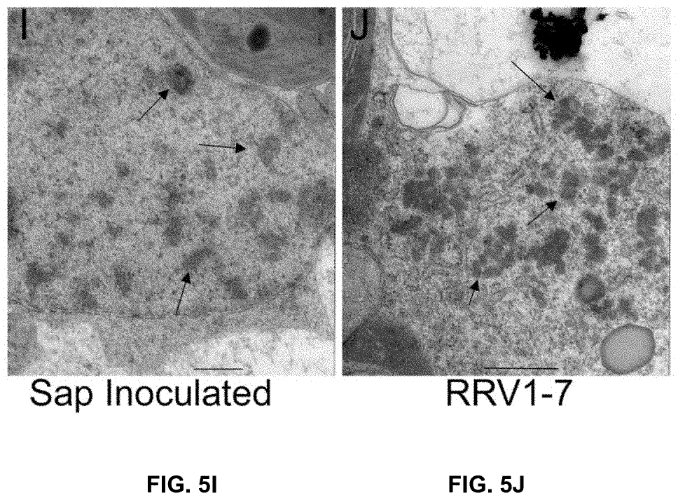

FIGS. 5A to 5J show experimental results of infectious clones in garden rose. The leaves that are outlined were selected for evidence of systemic virus movement and were analyzed by PCR. Images show infected rose leaves after inoculation and then PCR data that confirms infection.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A DNA polynucleotide encoding a Fimoviridae virus antigenomic RNA (agRNA) that is complementary to an RNA genome segment of the Fimoviridae virus.

2. The DNA polynucleotide of claim 1, wherein the Fimoviridae virus is an Emaravirus virus selected from the group consisting of a Rose Rosette Virus (RRV), Actinidia chlorotic ringspot-associated virus (AcCRaV), European mountain ash ringspot-associated virus (EMARaV), fig mosaic virus (FMV), High Plains wheat mosaic virus (HPWMoV), pigeonpea sterility mosaic virus (PPSMV), pea sterility mosaic virus 2 (PPSMV-2), raspberry leaf blotch virus (RLBV), redbud yellow ringspot-associated virus (RYRaV).

3. The DNA polynucleotide of claim 2, wherein the Fimoviridae virus is a Rose Rosette Virus (RRV).

4. The DNA polynucleotide of any one of claims 1 to 3, wherein the agRNA is agRNA1, agRNA2, agRNA3, agRNA4, agRNA5, agRNA6, agRNA7, or any combination thereof.

5. The DNA polynucleotide of any one of claims 1 to 4, wherein the agRNA is 70-100% identical to a polynucleotide that is complementary to any one of SEQ ID NOs: 4, 6, 8, 10, 12, 15, or 17.

6. The DNA polynucleotide of any one of claims 1 to 5, wherein the agRNA is operatively linked to a transcription control sequence and a self-cleaving ribozyme, wherein the agDNA is configured to produce viral transcripts with authentic 5′ and 3′ ends.

7. The DNA polynucleotide of claim 6, wherein transcription control sequence is a CaMV 35S promoter.

8. The DNA polynucleotide of claim 6 or 7, wherein the self-cleaving ribozyme is hepatitis delta virus ribozyme (HDR).

9. The DNA polynucleotide of any one of claims 1 to 8, wherein the polynucleotide is in a plasmid containing T7, SP6, RNA pol I, and RNA pol II promoters.

10. An agrobacterium cell transformed with the DNA polynucleotide of claim 9.

11. An infectious Fimoviridae virus composition comprising a plurality of the Agrobacterium of claim 10, wherein a first Agrobacterium comprises DNA polynucleotide encoding agRNA1, wherein a second Agrobacterium comprises DNA polynucleotide encoding agRNA2, wherein a third Agrobacterium comprises DNA polynucleotide encoding agRNA3, and wherein a fourth Agrobacterium comprises DNA polynucleotide encoding agRNA4.

12. The infectious Fimoviridae virus composition of claim 11, wherein a fifth Agrobacterium comprises DNA polynucleotide encoding agRNA5, wherein a sixth Agrobacterium comprises DNA polynucleotide encoding agRNA6, wherein a seventh Agrobacterium comprises DNA polynucleotide encoding agRNA7, or any combination thereof.

13. The infectious Fimoviridae virus composition of claim 12, wherein the ORF of agRNA5, agRNA6, agRNA7, or any combination thereof has been replaced with a transgene or non-coding RNA operably linked to an agRNA56, agRNA6, or agRNA7 viral promoter.

14. The infectious Fimoviridae virus composition of claim 13, wherein the transgene encodes a regulatory gene involved in transactivation of stress-responsive genes, stomatal movement, plant stress physiology, or a combination thereof.

15. The infectious Fimoviridae virus composition of claim 13, wherein the transgene provides drought tolerance, cellular protection/detoxification, transpiration control, or a combination thereof.

16. The infectious Fimoviridae virus composition of any one of claims 11 to 15, wherein the agrobacterium cells are suspended in an infiltration solution.

17. The infectious Fimoviridae virus composition of claim 16, wherein the infiltration solution comprises Silwet-77 surfactant.

18. A method for inoculating a plant, comprising administering to the plant the infectious Fimoviridae virus composition of any one of claims 11 to 17.

19. The method of claim 18, wherein the method does not comprise co-administering to the plant a viral replicase, nucleocapsid (NC) proteins, or silencing suppressor proteins.

20. The method of claim 19 or 19, wherein the agrobacterium cells are suspended in an infiltration solution, and wherein the infectious Fimoviridae virus composition is administered as a spray.


SEQUENCE LISTING (58 sequences)

SEQ ID NO: 1 - protein, 264 aa, Artificial Sequence
  Synthetic Construct
MAKMRISPELKKLIEKYRCVKDTEGMSPAKVYKLVGENENLYLKMTDSRYKGTTYDVEREKDMMLWLEGKLPVPKVLHFERHDGWSNLLMSEADGVLCSEEYEDEQSPEKIIELYAECIRLFHSIDISDCPYTNSLDSRLAELDYLLNNDLADVDCENWEEDTPFKDPRELYDFLKTEKPEEELVFSHGDLGDSNIFVKDGKVSGFIDLGRSGRADKWYDIAFCVRSIREDIGEEQYVELFFDLLGIKPDWEKIKYYILLDELF

SEQ ID NO: 2 - DNA, 4623 bp, Artificial Sequence
  Synthetic Construct
aagcttgcatgcctgcagtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaaagaatgcctctaccgacagtggtcccaaagatggaccccccacccacgaggaacatcgtggaaaaagaagacgttccaaccacgtcttcaaagcaagtggattgatgtgataacatggtggagcacgacactctcgtctactccaagaatatcaaagatacagtctcagaagaccagagggctattgagactttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagaatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggcctgacctgcaggtcgactctagaggatccccgggtcggcatggcatctccacctcctcgcggtccgacctgggcatccgaaggaggacgtcgtccactcggatggctaagggagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggaattcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacaggagatctcagtaaagcgctggctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaactcgagagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtctagtggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttt

SEQ ID NO: 3 - protein, 2276 aa, Artificial Sequence
  Synthetic Construct
MPPDVLSKFLRIAGTTLKNYTLTSKKKNIEKVYKQCTIDINFPKELVHLAEQILYMSPDPSQIDIVVTVIGLLELSRHDTLMMHVNDLLQSAGYNILGYDKPIKEVYPEINSILTPDVYFQIDDERYILELKVRNAKTDLDVFYHRYKNAIGNCGISIGVFNVSPSGFIETGDYRLTNLINIDPDRISDVTYCIELCSSLREKYGKYPEFFFFSHDRMLADNSEPFIETGYKNSVSEHADYKEISCLFGKHWPTIMDDIENFSLLTNPDVTTEKLLDANKNLYEDCNDRYDKFMEHLQYNYLNTESYGPTCLKDANLVSKLDKKNSEVYNITKKYKPAIYIPFFFSFFLDKYDYKRSLFYRSAFVDMKPAHDNYTNSVINLIDSIFNTVAIELIIKKNDEIDPAIYREVLSPEFCRHINDKTTKFKKIATISNVTSDTTILNNNAFSINHHVDKHMKDNIIGFKNKHYNEKVLKKDCISVADSEEDIITLKRYMGEIFNSEYHVGTYANELVSIDSDNMNTHICDLPEVCRTKYLDHLYSQHNIFKALISLNTVNSHKYRLVQTADPCTILITLPNSDTLKSAPLRYFTLTIISKKDLVSFEANRLLGVAHELIHGRNYNIILSKVISLDVTRLKLLNHSFGKYCLIMTYYSNMKKKIKHDIHFMIWILCQFITIASLTVTDTYKNFIKAVYSDYSNIDNLIDDKLESRPTTLGHAYIIKRCLDGIVKATQQLKVINQKKRNEEIDDMGELQNTGFNNNLSLKLPISNLSVDNPKEIIHEAFMLFYLGNKGLHGSPQELLNLYHTPYKFEKEYSEMLDKYGTILQETGNNSSMSISYNVMKKTSMVAYANLFNKKDAVRQSICSELEFDKPVLSLKQFSSTKSMVSNTKTDYVPAPSRLKDSTDIQILEKIINESIIEDDHQFMQLINSEVYRINAKRLKEEQKNPDMNVTLLPEIYIETIKGVRFIKIKRHQYTRMVNGGYIKQSNAKVFDEFYKLTEEYGLLSLKDAYKKMIHDDDLLIRIFYKDQRTADDREIYTGNAQTRLCLYPIEKTYKAICKHIPGEAITISGDQKQKKLLEQRLTLIKEKRQKIRDNKDAEIYSVSSDASKWSARDLFLKFIIVISTNPFLHPDEKWFLTFLCFRYYKRNIVLTDNIFNDMLNLVNVNSANTSYEEMTNDFKQNYFTVRSNWLQGNLNMISSFVHHCSTLYTEAMLKIFSDHNRLECNMTSMVHSDDSTYDFLICTGTKNQQKAINKGIINKTNIGKLIIALITFSNKFHCITLNEKKTYISTFYKEFLSTIIVGNELFFFYLADLLPLTSDTSYASPMQDLASYSGYINNAFSHACPLNMIKTSVILINHLTVSTYNMQYTSDKNPRFHIDSSDLPIQIYPRYKLPLNLAGMIPYYAADAFNILSDIIEKLNKFKILQTELIEDMLDVNTIEKYLEITRVNSPKVYKYIKSCILCMDYTQYERDDADPYNIIDYDLSQKSIINVVSLNKGLRLKKTYTYKKYLEHESEIRLKAAIHPEWCVKKPTDPDLIKANILQDYTNPNFRDGLIFSTPAIDYGRRIIGSNKNMYTISSHVMEKDKAKTIGTVYKDLSSKIDEVTLTANDLQRYLSLYLLSDKKILMAMQVFYSKVETVTMARPSYTKVIQPRSIYAEDFGKYSNTSLIQSLLTSRYCIINSIDPKSDKFIDICDYVLKRIGDTKIYENQEDIDDDYIAYYKFKYPNSPDIAIDVEPITDLGEVGFMAYKNKLKFMSLLVRYYNDIKRTLNNPSFNIPNYPSPSSLIMTIDSLLKKDEISTKVYLSNSKSTKYDEYLLTRYGMYAYNDFHIKYKLGYKIKIASSGKIASNLQTYRDTYEPLAFITKLIARNLNLYNRLKEHPEFMSGHYNSHELLNVLQNGKDINSSALLFMMGKIEFPRFITSLITDHRVYNHWLIPTNSEANDKNASLAYYMCQGNVMKVRTFTQNNVVVFSMMYYKYLRTDYGALESIKKKIASDYVELLRISKITSNITPTERYHVFNINEYGRYTNHFGDRVFNVCPIHYAKIIEVQPSYIEVDGNVKLILTITTNHEELAFDVRLRTYIDDEYYLNCLIDNLHIERNYILGYLCETNLFHKYPDYLKPVLIDLGPNQLISLMTGYTEFDIVTERINPQKFGNLIFFSNYFHEHGNSYMGNLCECVQLIALGQNIDVMASKDPDRFINSCNKIKLGETCADKVIKNYRANEIAPYHRLFITIMKYRHQSDPIEKIILFIILIFKFYIHDFIDMNDELEF

SEQ ID NO: 4 - DNA, 7026 bp, Artificial Sequence
  Synthetic Construct
agtagtgttctcccttaaatcattctaatctagacaaaatccaaaagaaagcaataaaggtctaaaagaaatagtgcgtgtaatttatctaaaattctagttcatcgttcatatctataaaatcatgtatataaaatttaaaaatcaagattatgaatagtataatcttctcaattgggtcactttgatgtctatatttcataattgtaatgaacaacctgtgatatggtgcaatctcattagcacgataattctttatcactttgtcagcacatgtttcacctaatttgattttgttacaactattgatgaatctatctggatctttagatgccatgacatctatattttgacctaatgctatcaattggacacactcacacagattgcccatataagaattaccatgctcatggaaataattgctaaaaaagatcaaattgccaaacttctgtgggttgattctttcagtgacaatatcaaattcagtgtaaccagtcattaatgatattagctggttaggtccaagatctattagtacaggcttcaagtaatcagggtatttatggaaaagattagtttcacatagataacccaatatataattacgttctatgtgtaaattgtctattaaacagtttaagtaatactcatcatctatataagtcctgagtcttacatcaaatgccaactcttcatgattggttgttatagttaaaatcagtttaacatttccatccacttctatataactaggttgaacttctatgattttagcatagtgtattgggcatacattaaagactcgatctccaaagtggtttgtatatcttccatattcatttatattaaacacatgatacctctcagtgggtgtgatattacttgtgattttagagattctcagcaactctacataatctgatgctattttcttttttattgattctaatgcaccgtaatcagttcgtaaatatttgtaatacatcattgaaaatacaaccacattgttttgagtaaatgttctaaccttcattacattaccttgacacatgtaatatgctagtgatgcatttttatcatttgcttcagagtttgttggtattaaccaatggttgtatactctatggtcagttattagcgaagttatgaatcttgggaattctattttacccatcatgaagagtaatgcactactattaatatctttcccattttgcaacacatttaacagctcatgggaattgtagtgaccactcataaattcagggtgttcttttaatctgttgtagaggttcagatttcttgctattaattttgttatgaatgctaatggttcgtatgtatctctataggtttgtaggttgcttgctattttaccactagatgcaatctttattttataacccaacttgtatttaatatgaaagtcattatatgcatacattccataacgtgtaagaaggtactcatcatacttagttgacttactgtttgataaatacaccttggtgcttatctcatctttctttagtaaagaatctattgtcataattaaagatgagggtgatggataattaggtatgttgaaacttgggttgtttaaagttcttttaatatcattataatatcgcactaataaagacataaactttagtttattcttgtatgccataaagccaacttctccaagatctgtgataggttcaacgtcaatggcaatgtcaggtgaattaggatatttgaatttataatatgcaatataatcatcatctatatcctcttgattttcatatatttttgtatcacctattctttttaaaacataatcacatatgtcaatgaatttatccgacttaggatcaatcgagttaattatacaatacctagaagttaataaagattgtatcaatgatgtattagaatatttaccaaagtcttcagcataaatgctccttggttgtataactttagtgtatgatggtcttgccattgtgacagtttctactttagaatagaaaacctgcatagccattagtatttttttatcacttaaaaggtataaacttaagtacctctgtaaatcgttagcagttagtgtcacttcatctatcttagaagataaatctttatatactgtacctatggttttagccttgtccttttccataacatgggaacttatagtatacatgtttttatttgaaccaataattcttctgccataatcaattgcaggagttgaaaaaattaatccatctctaaaattagggttagtataatcctgcaagatgtttgctttgatcaaatctggatctgtaggcttcttcacacaccattcaggatgaatagctgctttaagccgtatctcactttcatgctcaagatatttcttgtatgtgtatgtcttctttaatcttagacccttgtttaagctgacaacattgataatggacttttgactcaagtcatagtcaattatgttatatggatcagcatcatctctttcatattgtgtataatccatacacaggatacaagattttatatatttatacactttggggctattaactctggttatttcaagatatttctcaattgtgttaacatctaacatatcttcaattaactctgtctgtaggatcttaaacttatttagtttttctattatatcacttaaaatattaaatgcatctgctgcataatatggaatcatacctgctaaatttaatggcaacttgtatcttggatatatttgtattggtaaatcagagctatcaatgtgaaaccttgggtttttatctgatgtgtattgcatgttgtaagttgacacagtcaaatggtttattaaaataactgaagttttgatcatatttaaaggacatgcatgggaaaatgcattgttgatatatcctgaatatgatgctaaatcttgcataggtgaagcataacttgtatcagatgtcaaaggtaacagatctgccaaataaaagaaaaaaagttcatttcctacaattatagtagacaaaaattctttataaaatgtacttatataagtttttttctcatttaatgtgatgcaatggaatttattagaaaatgttattaaagcaataatcaattttccaatattagttttgtttattatacctttgtttatggctttttgttgattttttgtccctgtacagattaagaaatcatatgtggaatcatcagaatgaaccatagatgtcatgttacactctagcctattgtgatctgaaaatatcttgagcattgcttctgtatatagagtagaacaatgatgaacaaaagaagaaatcatgtttagattaccttgtagccaattacttctaactgtgaaataattttgtttaaaatcatttgtcatttcttcatatgatgtgtttgcactgttgacattaactaaattgagcatatcattgaagatgttatctgtgaggactatatttcttttataatatcgaaaacataaaaatgttaagaaccacttttcatcaggatgtaaaaatgggttggtagagatcacaatgataaattttaggaataaatctcgagctgaccattttgatgcatctgatgaaactgaatatatttcagcatctttattatccctaatcttctgtcttttttcttttatcaaggttaaccgttgttctaatagttttttttgtttttgatccccacttatagttatagcttcaccaggtatgtgtttacatatagctttgtatgttttctctattggataaagacacaatctagtttgtgcattaccagtatagatttcccgatcatcagctgttctttggtctttataaaatattcttataagtaggtcatcatcatggatcatctttttataggcatcttttaagctcaataaaccatactcctcagttaacttgtaaaactcatcaaagactttagcattactctgttttatgtaaccaccatttaccattcttgtatattgatgtctttttatctttataaatctaacaccctttattgtttctatgtatatctcaggaagaagtgtaacattcatatcagggttcttttgctcctcctttaatcgtttagcattaatcctataaacttcagaattaattagttgcatgaattggtgatcatcttcaattatagattcattaataatcttctctagtatctgtatatctgtactatcttttaaacgactaggtgctggcacataatcagttttggtgtttgataccatagattttgtgctagaaaactgcttaagtgataatactggtttatcaaattctaattcactacatatgctctgtcgaactgcatcctttttgttaaataaattggcatatgcaaccatagatgtttttttcatcacattgtatgaaatggacatagatgaattattaccagtttcttgtaatattgtgccatatttatctaacatttcactatattccttttcaaatttatatggtgtatgatataagttcaataattcttgtggagagccatgtaagcctttattacctagataaaaaagcatgaatgcttcatggattatttctttgggattatcaacactgagattagatattggtagctttagactcaagttgttgttgaaacctgtattttgtaattcacccatatcatctatttcttcatttctttttttttgattgatcactttgagctgttgtgttgccttaacaataccatctagacacctttttattatgtatgcatgtcctagtgttgttggtctagattctaatttatcatctataagattatctatattagaataatccgaatatacagccttaatgaaattcttatatgtatctgtaacagttaatgatgctattgttataaactggcatagtatccatatcataaagtgtatatcatgtttgatcttttttttcatgttggaataataagtcataattaggcaatactttccgaaagaatggttaagtagtttaagtctagtcacatccaaactgattacttttgatagaatgatgttgtagtttcttccatgtatcaactcatgtgctacacctaataggcgatttgcttcaaaacttacaaggtcttttttagatataatagttagagtaaagtaccttaaaggggctgatttaagtgtgtcagagtttggcagcgttatgagtatagtgcatggatctgctgtttgtaccaacctatacttgtgtgaattgacagtatttaacgatatcaaagctttaaagatgttatgttgactatataagtgatctaaatatttagttctgcaaacttctggcaaatcacatatgtgtgtattcatattgtctgagtctatagagactagttcattggcataagttccgacatggtattcactattgaatatttcacccatgtatctttttagagttattatatcctcttcactgtcagcaactgatatacaatctttcttcaacaccttttcattgtagtgcttatttttgaaacctataatattatctttcatatgtttgtccacatgatggttgattgagaatgcattgttgtttaagattgttgtatcactagtaacattggatattgtagcaatttttttgaattttgtagttttgtcatttatatgtcgacagaattcaggtgataatacttctctataaattgcaggatctatttcgtcattttttttaataattaactctattgcaactgtattgaaaattgaatctataagatttataacagaattagtgtaattatcatgtgcaggtttcatatctacgaaagcactcctataaaatagagatctcttgtaatcatacttatccaaaaaaaaagaaaaaaaaaaaggtatatatattgcaggtttatattttttagttatattgtaaacttcagaattcttcttatcaagtttggaaaccaaattggcatcttttaaacatgtaggtccatatgattctgtgtttaggtaattatattgtagatgctccatgaatttatcatatcgatcattgcaatcttcatatagatttttattggcatccaacagtttttctgtagtaacatcagggtttgttaacaaactgaagttctctatatcatccattattgtgggccagtgtttaccaaatagacaactaatttccttgtaatcagcatgttcagagacactattcttgtaaccagtttcaatgaatggttctgaattatctgctaacatcctatcatgtgaaaaaaaaaaaaattcaggatacttcccatatttttcacgtagtgaagaacatagttcaatacagtaagtaacatcactgatacgatctggatcaatgttgataagattagtgagccgataatcccctgtttctatgaagccagatggtgagacattaaaaacaccaatacttataccacagttacctatagcatttttatatctgtgataaaatacatccaaatcagttttggcatttctaactttaagctcaagtatatacctttcatcatcaatctggaaatatacatctggtgttaggatactgttgatctcaggatatacttctttgattggcttatcataacccaaaatgttataaccagcagattgaagtaagtcatttacatgcatcattaatgtgtcatgccttgacaattctagcaatccaatgacagtgactactatatctatctgacttggatcaggtgacatatacaatatttgctcagcaaggtggaccaattctttggggaaattaatatctattgtgcactgcttatagactttctctatattttttttctttgatgttaaagtgtagtttttcagtgtggtccctgcaattcgaagaaatttagaaagcacatcaggtggcattgcattacctgaccgaatcttagtgatcgcatcattgtaaatcaaacctttttttagcttccataattgcttttgaatttaaattgtatttaagggagttcactact

SEQ ID NO: 5 - protein, 645 aa, Artificial Sequence
  Synthetic Construct
MKMSLRNTLVAIGVLLLSCGTFVKEVKNHFHAEVDVCTCTPNIEKLAKEFVVCFPGCQIKPVNSHLFNETCSYMEQVTVTICNGDRYISTKPSIIAHKSYVWETYLNKAWKVIVSLFVWLMLVISKTPVLCIIALLNKALHKIFPKRLWKCSRCDSEYLFSHLDCPTPSFKNKTDYNFLFYILLVLSVVTTIAKADDNQYNYYLHSNVTEIQVLDKEHYQQDFDVNGYLYTITILNSHLVMEVINISDIYIPVSHKIEEVTYSCDGAVECLADLKKKTTNDNTWYLKKVHDGFSCLTTTATVCGACTSRLHYIGTKATTAKVSPYIDIQIKHGNKTEVIEIREFSKYIHLPYYVKPLAPILVESNEMLISDHKVFYGQFCDQPSYGCFGPNYKKDGQMYVLNSPMVKDPMTHDREVILVHCTDPGNTDMNSLKSTDFVYQNQTIIRPFEFGMISMGIPNIGKLIGDFCEKPADVISVNVNGCYDCQLGIEIVVKFNINTKCAQIKCDVGKVSYEYFVDSSSNTLTLHSFYDREEVFIKCNKYSGTFRLEHSKDTDYYKTSNEVHGSATYDFNLLKHIPNLLTNFKTLILSIILVIMVVYMLISISKHTIKHFLLLKKKRKLSYNKKNDISEEEVIESIIIGDPDI

SEQ ID NO: 6 - DNA, 2245 bp, Artificial Sequence
  Synthetic Construct
agtagtgttctcctcatataaacgcagaaattgacaaaagcttgaaaacaattaattgatgagaatatatctcagttcagcagatgttactccattatgaaatgaagcaacacaattggtaatttgaggtaatgttacaaatcaccattgcaggttgttgcattataactctgcagcgtgctgaacaagggtggaccattccacaaaggtataaaaaagctttggaaagaaagggtagagccaaacttgatttttgactatatatctggatcgcctattataatagactcaatcacttcttcttcacttatatcattctttttgttatatgataattttctcttctttttcaataataggaaatgcttgatggtatgtttactaatactaattaacatatataccaccattattaccaaaattatgctaaggatcaatgttttgaagttggtaagtaaattaggaatatgtttcaataggttaaaatcatatgtagcactgccatgcacttcattacttgtcttgtaatagtcagtgtccttactatgctctaatctgaatgtgccactatacttattacacttgatgaaaacttcttccctatcataaaaagagtgcaatgtaagtgtattactactagagtctacgaaatattcataacttacttttccaacatcacatttgatttgtgcacacttagtatttatattaaactttaccactatctctataccgagttgacaatcataacaaccattgacattcactgatatgacatcagcaggtttttcacagaagtcacctattagtttaccaatgtttggtatgcccatagaaatcattccaaattcaaatggtcgtattattgtttgattctgatagacaaaatcagtggacttcaagctattcatatcagtgttaccagggtcagtgcaatggactaatattacttctctatcatgtgtcataggatctttcaccataggtgaattcagtacatacatttgtccatcttttttatagttcgggccaaaacaaccatagcttggttgatcacagaattgtccataaaacactttgtggtcagaaataagcatttcattagattctactaagataggagctaaaggtttaacataatatgggagatgaatatactttgagaactctcttatttcaataacttcagtcttattaccatgcttaatttggatatcaatataaggagatactttagctgtagtagctttggtgccaatataatgtaatcttgatgtacatgcaccacagacagttgcagtagttgttaaacagctgaagccatcatgaacctttttaagataccatgtattatcatttgtggtcttctttttgagatctgctaagcattcaacggctccatcacaactatatgttacttcttctatcttatgtgacactggtatataaatatcagatatattgataacttccattaccagatgtgaatttagtatagtaatagtatatagataaccattcacatcaaaatcctgttgataatgctctttatctaagacttgtatttcagtaacattactgtgtaaataataattatactgattgtcatctgctttagcaatagttgttacaactgataagactaacaaaatgtagaataaaaagttgtaatctgttttattcttgaaacttggagttggacaatctagatgggagaacaaatattcagaatcacatctactacacttccataaccttttaggaaagatcttgtgcagagctttgttaagtaatgctatgatacacaaaacaggtgtctttgatatcactaacatcaaccaaacaaacagacttactatcactttccaggctttgttcagataagtttcccacacataagacttatgtgctatgatagaaggtttggtagatatatatcgatccccattacagatagtgactgtcacttgttccatgtagctgcaagtttcattaaacaggtgagaattaacaggtttgatttgacaacctggaaaacagacaacaaattcctttgccagcttttcaatgtttggtgtacatgtgcacacatctacttcagcatggaaatggtttttcacttcttttacaaatgtcccacagctaagtagcagaactccaatcgcaactagggtgtttctcagactcatcttcatttttttcaagagtttttcaagttgactagttttatgaggagttcactact

SEQ ID NO: 7 - protein, 316 aa, Artificial Sequence
  Synthetic Construct
MAPKNEFKKPSKIENPGTSTSNTLAADTVIIGADQKLRVLKLTNVQNKKLVKPESSELDPVYIEPTPFTSEIQSPNNFSLAPYRNFCNVGIAVSYLSRSLEIKEQLQIRNVFIRTSNKDSLTIVKDLNESDVLNVVSFNKACAILSAGVLKHVFKEEFDWTLKKYVPTTVSSRTAPDETVVNRLAGQMGMDSKNPYYWMIVPGYEFLYELYPAEVLAYTLIRLEFRKNLNIPDDMTDSDIVNSLVMKMNRIHKLETTPFDDAISLIGREEFALAYVELARDIGTTSKSKRNDEAIGKFKELIKNFAPALAADRGAQ

SEQ ID NO: 8 - DNA, 1544 bp, Artificial Sequence
  Synthetic Construct
agtagtgttctcccataatttatctaagctaacgaaaaactttttaaaaactcaatatattggtttctaaagcctaatagcgtttatttattgatttatgaaatatataaacggatagaggagttttatatttacatctatttacaacttactaactaggtggaacatctctttttattataaacaatctagtacatatattagttaagctacataaaatattagtagatatatatttagatttgtagcttattgaaacttattatagactagttactacttatcaatcattatatttaatacaatcatcttttttgtttattttttttgctttttatcgccttttcttcttttttttgttgttttccattttttattttttttgattttttttgtttttttgttttttttgttgttttttgtttttttattaatataatattcatatttaataattacttatatacaccttcagttttataaaaaactgatatttattgtgcacctctatcagcagctaaagcaggagcaaagttcttgatcagttctttgaacttgcctatagcttcatcattcctctttgatttgcttgttgtaccaatgtctctagcaagttcaacatatgcaagggcgaattcttctcttccaatgagacttatagcatcatcgaaaggagtagtctctagcttgtgaatacgattcatcttcatgactaaagaattgacaatgtcactgtcagtcatgtcatcaggaatattcaagttttttcggaattctaaacgaataagtgtataagccaacacttcagctggatagagctcatacaaaaactcataccctggtacaatcatccaataataaggattttttgagtccattcccatttgtcctgccagcctgttaacaactgtttcatctggtgcagttcttgatgaaacagtagtaggtacatacttcttaagagtccagtcaaactcttctttgaacacatgctttagaacaccagctgataaaattgcacatgctttgttaaacgaaacaacatttagaacatcagactcattcaagtctttaacaattgtcagagaatccttgtttgatgttcttataaacacatttcttatttgaagctgctccttgatttccagggacctagaaagataagaaacagctatgccaacattacagaagtttctgtaaggtgctagactaaaattgttgggactttgaatctctgaagtaaaaggtgtaggttcaatataaactgggtccaattctgaactttcaggctttactagcttcttattttgaacatttgtcaacttgaggacccgaagcttctgatcagctccgatgataacagtatcggctgcaagagtgttggatgtgcttgttccaggattctcgatcttcgaaggcttcttgaactcattctttggtgccattgtagtattctctaaaaacgctgttttaatgtgaactccaagtattagatttttaagaaaagtttatcgatcgattgataattatgggagttcactact

SEQ ID NO: 9 - protein, 361 aa, Artificial Sequence
  Synthetic Construct
MAFSSILFFLIAMAINVDSMKPTKISNFDVTTGEFEGIESITELTWNTFKKMGVKTKLTLEAKTQLLPISVYNEMYKFYMHLKQPMTRIASVALFWTPTSKRFNEMATLILMDERFRDDGIKQGKKELLAQGKLNLDLMGTVITAVPFDPNFQQFVTGSLDFATDTKDINKIKFYISFPDTRMGETQSTAGFIDLSWKTLPDENGVYENIQWNVFTFPLSLPPEVSMMSGKSNFLKVKNYLDRKYNERKEALLQLNEISNQLIWGSDQGLNKVRAKLKEDTNTSTQLLIDSEKREEAIKLRLIQDEISEAKAELSSALSGIDLNAINAAKTKLAAVLRMHNLAENIPQEMDYGYIKIGEAN

SEQ ID NO: 10 - DNA, 1541 bp, Artificial Sequence
  Synthetic Construct
agtagtgttctccttacatatcaaatcgatctacacaaaatttcttaaacaaacaataagttaagtgatggaatctagactagcatttcccaaatttatttccatttgtatatgtcggtactctatttgggttgcaagcaacctctttttttggttttttgattttatgttttttttgtatttttttactttttttggaagcttaaacaacttaaagataactatattgcttaaaacactttatttacatacatagctatatagatttaaattttaaaactagttttaaaatatgctcttgacactgcaacttttttacaaacttagtaggcttcttaattttactaacggtgcttaataagcttgtacatcttagttggcttcaccaattttaatgtagccgtagtccatctcttgagggatattttcagctaaattgtgcattcttaatacagctgccagtttagtttttgcagcatttatagcatttagatcaattcctgacagtgcagagcttaattctgcttttgcttcagagatttcgtcctgaatcaatctaagcttaatagcttcttctcttttctcactatctatcaacagctgtgttgatgtgtttgtatcttctttgagcttggccctaaccttgttaagaccttgatcacttccccaaataagttggtttgagatctcatttagctgcagcaaagcctctttcctttcattgtattttctgtcaagataattttttactttcaaaaaattactctttcctgacatcattgaaacctctggtggaagacttagagggaaagtgaaaacattccattgaatattctcatagacaccattctcatctgggagagtcttccaagacaagtctatgaatccagctgtagattgagtttcacccatcctagtatcagggaatgatatataaaacttgattttattgatatcttttgtatcagttgcaaaatcaagtgagcctgtcacgaattgttggaaatttggatcaaatggtacagcagttataacagttcccataagatcaagattaagcttcccctgagccaacaattctttttttccttgtttgataccatcatccctaaatctttcatccatcaatatgagtgttgccatctcattaaatcttttagaagttggagtccaaaataaagctacacttgcaatccttgtcatcggttgttttagatgcatatagaatttatacatctcattatacacagatataggtaatagctgtgttttagcttccaatgttaactttgttttaactcccatcttcttaaaagtattccaggttagctcagtgatggattcaatcccctcaaattccccagttgtcacatcaaagttgctgatcttcgtaggtttcatgctatcaacattaatagccattgcaatcagaaaaaacagtattgaagaaaaagccatcgtacttaggtgttctcactcacagattttagtttttttaagagtttttgaaaacagctttaatttgtaaggagttcactact

SEQ ID NO: 11 - protein, 467 aa, Artificial Sequence
  Synthetic Construct
MEKIIPYVRPDPERVDFSNHEKLPKLPCLGDFYGPIYKLIRENFNSCNCVEKKLFYGEYTRIPCNSSIGYIEINRGEMPTVIATVLNRGTVRLEVLPEDVVNALKSIVYNYVIENYENLEMVTAVIKNAAAVKNYFIMVKIIDKQWIINETKKNWANIIGSTYLAQKELIISHYLRMKSFYYRIVCNSRYEKSVYSFIDENKHLSNEIIPSLDIMLRTYNIHVFKYVKEHIQYCKKMLDYDIPFDCNKIKESDFMYGEFFPLDVKTAIYEDVTIRLREVRIENSEVDVLKKPIIEYIDKEILLNDIALNKAVDEKIKYLPGDNEIEDMFYYGLPGSIKTVVNNINSSMKKNISDDTKIDDIDYIPNYPFKSSYKKEVYNLYKLKNSDLKMSHLTLHYICLYLYMAKVSAIRTGYSENIGTVEVLERAREFAKVNVSHLRNRLLFLHQNRISLKDGCMQIFLKVNWTQ

SEQ ID NO: 12 - DNA, 1665 bp, Artificial Sequence
  Synthetic Construct
agtagtgttctcccacaaaaatatcaaattcaatgaaaactttcttaagctaaccaagtggcctaaatcaacaacaagacgtgcaagttaattaaataaccaagctaatcatacaattgtataacaagttccaaattgctacctatttcattgtttaagatgaaatatattgccatcattggaacaaatatattgtaactctattgagtccaatttactttcaaaaagatttgcatgcaaccatccttaagagatattcgattctgatgtaagaaaaggagtctgtttcttaagtgactgacattaacttttgcgaattcccttgctctctctagtacttcaactgtccctatattttcactgtaacctgttctaatagctgaaactttggccatgtataaatataaacagatataatgtaatgttaggtgagacatctttaaatcactgtttttcagtttatataagttgtaaacctcttttttgtaactagatttgaatgggtaattaggtatgtaatctatgtcatcaatttttgtatcatctgatatattctttttcatggagctattgatattgttgacaactgttttaatactaccaggtaagccataataaaacatatcttcaatctcattatcaccaggcaaatacttgatcttttcatcaacagctttattcaacgctatatcattcaaaagaatttctttatcaatatattcgataattggttttttcaatacatctacttctgagttctctatcctcacttctcttagtcttattgtaacatcctcataaattgctgttttaacatctaaagggaagaactcaccatacataaaatctgattctttgattttgttgcaatcaaatggtatatcataatctaacatctttttacagtattggatatgttctttgacatatttaaatacatgtatgttataagtcctaagcatgatatcaagacttggaatgatctcattagataagtgtttgttttcatctataaaagaataaactgatttctcatatcttgaattacatacaatcctataataaaatgacttcattctgaggtagtgacttatgatcaattctttttgtgcgagatatgtagagcctattatattagcccaatttttctttgtttcatttattatccattgtttatcaatgattttaaccattataaagtaatttttgactgctgctgcattttttatcactgctgttaccatttctaagttttcataattttctataacatagttatacacaatgctttttaatgcattaacaacatcctcaggtagtacttcaagcctcacagtccctctgtttagaactgtagcaataacagttggcatttcacctctattaatctctatgtagccgatagatgaattacatggtatgcgagtgtactcaccataaaacaatttcttctcaacacagttgcagctgttaaaattttctcgaatcagcttgtatataggtccatagaagtcaccgagacatggcagcttgggtagtttctcatggttactaaagtcaactctttcagggtctggacgaacgtaagggatgattttttccatcactggtgcaagctttaaaagagttttttgttaatcgaaattattgtgggagttcactact

SEQ ID NO: 13 - protein, 44 aa, Artificial Sequence
  Synthetic Construct
MLVLHQSWLLMINMILSLKKLCIIIIKTKTLIRCNKYWYCSISL

SEQ ID NO: 14 - protein, 233 aa, Artificial Sequence
  Synthetic Construct
MEQKTFMKTYKALVNNKFSDHFCELETAIFNSMIEILDNDEEYSEVIVLHDAYLKNQLEFSEINYQTQQIEISGMKIQKKSLRKFLRYLQITCSLFGLTSVLPESIHHDFHIFGTLVRGERIFPYIPDHSQYLLMSSNIVKAIVKQVVLGYDPEDTIIKAQSQQQEDVNYLESTVAFLFHVHAIAYTTGRLNNKLPLETHQINNTINNASASSVMVIDDKYDTKLEETVYHNH

SEQ ID NO: 15 - DNA, 1402 bp, Artificial Sequence
  Synthetic Construct
agtagtgttctccctataaacttcagcagctttcaaaaaactttcttaaactataaaaatttggtgatttggttctaaaaacgttcgtattgccatttataaaaattgtagcaatatatctttgttcaaattccaaatttaatatcggttattggtcatattatactttttcaagcataacataacgttttttttgttagatatttactgtttcaaaaatattttacaggcagcaaatatctgattttttgtttttttgttttttttgttttttttgttgagaaatgtttcctctttttatgcttttttttggtcttttgtgtttttatctggactattatattcagaatattgttatctatgccatttgaagaagattttttgaagaaataataaagcttgtggtaactattaatatatctattatttattggttactatattataatatccatctgatctttgttttgattttttttagtttatagactcttttgtgctttttttcgtcatttttctttttttatatataaaacataataaaacttacaagtgcctgtaggctattgtaattaagtgacaagcttattgaacaataccaatacttattgcatcttatcaatgtcttggttttaatgattatgatacacagtttcttcaagcttagtatcatatttatcatcaataaccatgactgatgaagcactagcattatttatagtattattaatctgatgtgtttccaatggaagtttattgttcaatctgccagtggtataagcaatagcatgaacatgaaacaaaaaagcaacagttgattctaaataattcacatcttcttgttgttgagattgtgccttaattattgtgtcttctggatcataacctaggaccacttgttttacaattgctttcacgatatttgaagacatcaataagtactgtgaatggtctggtatatatggaaatattctttcacctctcacaagagtaccaaatatgtggaaatcatgatggatgctttcaggcaatacacttgtcaatccaaacaaagaacatgtgatttgtaaatacctcaaaaactttctaagtgatttcttttgtatcttcatcccgcttatttcaatttgctgtgtttgatagttgatctccgaaaattcaagctgattttttagatatgcatcatgtagaacaatcacctcactatactcttcatcgttgtctaggatctcaatcattgagttgaaaatagcagtctccaattcacagaaatgatcagagaatttgttattgacaagagctttgtatgttttcatgaacgtcttttgctccatggttgacctaaatatgatttgctttaagagtttttcaagtcgcttggttttatagggagttcactact

SEQ ID NO: 16 - protein, 465 aa, Artificial Sequence
  Synthetic Construct
MDSIIPYTRSDSEVIDFSNYEKLGKLNNLGAFYGTIYSNIKTMFNADVSTEKKFFYGSHNKIPKHSSICYLEINHKKIPNTVASVFNKTQVEVETLPEDVCLALQSILYNYVIENFNNLDTLSAVIMHHKLVRRYFIMIKIVDKQWMIDEVKRNWANILSSTYLSQREVILSHYTSMTAFYYKIVKNSDTEKSVYSFLDDNKHLLNHVLPSLDVDFKLYNAQIIKYVKSHLQICKDKSDYNLAFDYTTISESDSIYGEKFPADVLAAIHIDVTAKLREVKVNRSEEDVLKKPIIDCVDDVVVLNKVTFWKHTDETVSYKPGDDEIEDTFYYGIPDTIETVIDHLEDALEKNIHDVDDINNIDYIPQFPFKNSFKREIFTALKVRNDTVKVSHLTLHYICLFLYISKVASLKIDTGCYTAHSAVLERARLFAKTNVSYLRNRLLFLNQNRKQFKNECVQISLKFNM

SEQ ID NO: 17 - DNA, 1649 bp, Artificial Sequence
  Synthetic Construct
agtagtgttctcccacaaattaatcaaaaaactgataaaagcttgaaaactctaatataagtggaattaacaattcatagatgactaaattttattctaattcgctaattatttcactttttaaggtgaaatacttcgccataattagaacttaatggaaataattattatttaaaagatagaataccaattacatattgaattttaagctaatttgtacacattcatttttgaattgcttcctattttgatttagaaaaagtagcctattccttaggtagctcacattagttttagcgaataatctggctctctctaaaacagctgaatgtgctgtataacaaccagtatcaatctttagactagcaactttagatatgtataaaaatagacaaatataatgaagagtcagatgtgagactttaacagtatcattcctaacctttaatgcagtgaatatttcacgtttgaagctgttcttgaatggaaattgaggtatatagtctatattgtttatatcatctacatcatgaatatttttttccaaagcatcttctagatgatcaatcacagtttctattgtatctggaataccatagtaaaaggtatcctcgatttcatcatcacctggtttatatgagactgtctcatcagtatgtttccaaaaagtaactttgtttagaacaactacatcatctacacaatcaattattggtttcttcaatacatcttcttcacttctattgacttttacttctcttagtttagctgtcacatcaatatgaatagctgctaagacatcagctggaaatttttcaccatatatagagtctgattcagaaattgtagtgtaatcgaaggcaagattgtaatctgatttgtctttacatatctgtaagtgacttttgacatattttattatctgtgcattatacagcttaaaatcaacatctaagcttggaagaacatgattcagaaggtgtttattatcatccaagaaactgtaaactgatttctcagtatcagaatttttcactatcttgtaatagaatgctgtcatgcttgtgtaatgacttaaaattacctctctctgtgaaagatatgttgagcttagaatgttagcccaattcctcttcacttcatctatcatccattgtttgtcaactatttttatcattataaagtatcgtctgaccaacttatgatgcattataacagctgacagagtatctaagttgttaaaattctctataacatagttatatagaatgctctgaagtgctaaacagacatcttcaggtaatgtttcaacttcaacttgagttttgttaaacactgatgctacagtgtttggtatttttttatgattaatctccaagtaacagatagatgaatgtttcggaatcttattgtgtgaaccataaaagaattttttctcagtactgacatcagcattgaacatagtcttgatattgctatatatagttccatagaaagcaccaagattgttaagcttacctagcttttcataatttgaaaagtcaatgacttcagaatctgaacgcgtgtaaggtatgattgaatccatcacttgttgagtttttttaagctttttttcaaaatctatttaattgtgggagttcactact

SEQ ID NO: 18 - DNA, 2270 bp, Artificial Sequence
  Synthetic Construct
agtagtgaactccttacaaattaaagctgttttcaaaaactcttaaaaaaactaaaatctgtgagtgagaacacctaagtacgatggctttttcttcaatactgttttttctgattgcaatggctattaatgttgatagcatgaaacctacgaagatcagcaactttgatgtgacaactggggaatttgaggggattgaatccatcactgagctaacctggaatacttttaagaagatgggagttaaaacaaagttaacattggaagctaaaacacagctattacctatatctgtgtataatgagatgtataaattctatatgcatctaaaacaaccgatgacaaggattgcaagtgtagctttattttggactccaacttctaaaagatttaatgagatggcaacactcatattgatggatgaaagatttagggatgatggtatcaaacaaggaaaaaaagaattgttggctcaggggaagcttaatcttgatcttatgggaactgttataactgctgtaccatttgatccaaatttccaacaattcgtgacaggctcacttgattttgcaactgatacaaaagatatcaataaaatcaagttttatatatcattccctgatactaggatgggtgaaactcaatctacagctggattcatagacttgtcttggaagactctcccagatgagaatggtgtctatgagaatattcaatggaatgttttcactttccctctaagtcttccaccagaggtttcaatgatgtcaggaaagagtaattttttgaaagtaaaaaattatcttgacagaaaatacaatgaaaggaaagaggctttgctgcagctaaatgagatctcaaaccaacttatttggggaagtgatcaaggtcttaacaaggttagggccaagctcaaagaagatacaaacacatcaacacagctgttgatagatagtgagaaaagagaagaagctattaagcttagattgattcaggacgaaatctctgaagcaaaagcagaattaagctctgcactgtcaggaattgatctaaatgctataaatgctgcaaaaactaaactggcagctgtattaagaatgcacaatttagctgaaaatatccctcaagagatggactacggctacattaaaattggtgaagccaacaggcctatggtttctaagggtgaggaactcttcaccggtgttgttcctatcctcgtggaactcgatggtgatgttaacggacacaagttctctgtgtctggtgaaggtgagggtgatgcaacttacggaaagctcaccctcaagttcatctgtaccactggaaagctccctgtgccttggcctactcttgttactactctcacttacggtgtgcagtgcttctcaagataccctgatcacatgaagcagcacgatttcttcaagtctgctatgcctgagggatacgtgcaagagaggaccatcttcttcaaggatgatggaaactacaagaccagggctgaggtgaagttcgaaggtgatactctcgtgaacaggatcgagcttaagggaatcgatttcaaagaggatggtaacatccttggacacaagctcgagtacaactacaactcacacaacgtgtacatcatggcagataagcagaagaacggaatcaaggttaacttcaagatcaggcacaacatcgaggatggttctgtgcagctcgctgatcattaccagcagaacactcctatcggagatggacctgttctcctccctgataaccactacctttctacccagtctaagctctctaaagatcctaacgagaagagggatcacatggtgctcctcgagtttgttacagccgctggaatcaccctcggaatggatgagctttacaagtgacccggggatgtacaagcttattaagcaccgttagtaaaattaagaagcctactaagtttgtaaaaaagttgcagtgtcaagagcatattttaaaactagttttaaaatttaaatctatatagctatgtatgtaaataaagtgttttaagcaatatagttatctttaagttgtttaagcttccaaaaaaagtaaaaaaatacaaaaaaaacataaaatcaaaaaaccaaaaaaagaggttgcttgcaacccaaatagagtaccgacatatacaaatggaaataaatttgggaaatgctagtctagattccatcacttaacttattgtttgtttaagaaattttgtgtagatcgatttgatatgtaaggagaacactact

SEQ ID NO: 19 - DNA, 2330 bp, Artificial Sequence
  Synthetic Construct
agtagtgaactccttacaaattaaagctgttttcaaaaactcttaaaaaaactaaaatctgtgagtgagaacacctaagtacgatggctttttcttcaatactgttttttctgattgcaatggctattaatgttgatagcatgaaacctacgaagatcagcaactttgatgtgacaactggggaatttgaggggattgaatccatcactgagctaacctggaatacttttaagaagatgggagttaaaacaaagttaacattggaagctaaaacacagctattacctatatctgtgtataatgagatgtataaattctatatgcatctaaaacaaccgatgacaaggattgcaagtgtagctttattttggactccaacttctaaaagatttaatgagatggcaacactcatattgatggatgaaagatttagggatgatggtatcaaacaaggaaaaaaagaattgttggctcaggggaagcttaatcttgatcttatgggaactgttataactgctgtaccatttgatccaaatttccaacaattcgtgacaggctcacttgattttgcaactgatacaaaagatatcaataaaatcaagttttatatatcattccctgatactaggatgggtgaaactcaatctacagctggattcatagacttgtcttggaagactctcccagatgagaatggtgtctatgagaatattcaatggaatgttttcactttccctctaagtcttccaccagaggtttcaatgatgtcaggaaagagtaattttttgaaagtaaaaaattatcttgacagaaaatacaatgaaaggaaagaggctttgctgcagctaaatgagatctcaaaccaacttatttggggaagtgatcaaggtcttaacaaggttagggccaagctcaaagaagatacaaacacatcaacacagctgttgatagatagtgagaaaagagaagaagctattaagcttagattgattcaggacgaaatctctgaagcaaaagcagaattaagctctgcactgtcaggaattgatctaaatgctataaatgctgcaaaaactaaactggcagctgtattaagaatgcacaatttagctgaaaatatccctcaagagatggactacggctacattaaaattggtgaagccaacaggcctcagcttctgaactttgatctgctcaagctggcgggcgatgtggaatccaacccaggcccaatggtttctaagggtgaggaactcttcaccggtgttgttcctatcctcgtggaactcgatggtgatgttaacggacacaagttctctgtgtctggtgaaggtgagggtgatgcaacttacggaaagctcaccctcaagttcatctgtaccactggaaagctccctgtgccttggcctactcttgttactactctcacttacggtgtgcagtgcttctcaagataccctgatcacatgaagcagcacgatttcttcaagtctgctatgcctgagggatacgtgcaagagaggaccatcttcttcaaggatgatggaaactacaagaccagggctgaggtgaagttcgaaggtgatactctcgtgaacaggatcgagcttaagggaatcgatttcaaagaggatggtaacatccttggacacaagctcgagtacaactacaactcacacaacgtgtacatcatggcagataagcagaagaacggaatcaaggttaacttcaagatcaggcacaacatcgaggatggttctgtgcagctcgctgatcattaccagcagaacactcctatcggagatggacctgttctcctccctgataaccactacctttctacccagtctaagctctctaaagatcctaacgagaagagggatcacatggtgctcctcgagtttgttacagccgctggaatcaccctcggaatggatgagctttacaagtgacccggggatgtacaagcttattaagcaccgttagtaaaattaagaagcctactaagtttgtaaaaaagttgcagtgtcaagagcatattttaaaactagttttaaaatttaaatctatatagctatgtatgtaaataaagtgttttaagcaatatagttatctttaagttgtttaagcttccaaaaaaagtaaaaaaatacaaaaaaaacataaaatcaaaaaaccaaaaaaagaggttgcttgcaacccaaatagagtaccgacatatacaaatggaaataaatttgggaaatgctagtctagattccatcacttaacttattgtttgtttaagaaattttgtgtagatcgatttgatatgtaaggagaacactact

SEQ ID NO: 20 - DNA, 2584 bp, Artificial Sequence
  Synthetic Construct
agtagtgaactcctcataaaactagtcaacttgaaaaactcttgaaaaaaatgaagatgagtctgagaaacaccctagttgcgattggagttctgctacttagctgtgggacatttgtaaaagaagtgaaaaaccatttccatgctgaagtagatgtgtgcacatgtacaccaaacattgaaaagctggcaaaggaatttgttgtctgttttccaggttgtcaaatcaaacctgttaattctcacctgtttaatgaaacttgcagctacatggaacaagtgacagtcactatctgtaatggggatcgatatatatctaccaaaccttctatcatagcacataagtcttatgtgtgggaaacttatctgaacaaagcctggaaagtgatagtaagtctgtttgtttggttgatgttagtgatatcaaagacacctgttttgtgtatcatagcattacttaacaaagctctgcacaagatctttcctaaaaggttatggaagtgtagtagatgtgattctgaatatttgttctcccatctagattgtccaactccaagtttcaagaataaaacagattacaactttttattctacattttgttagtcttatcagttgtaacaactattgctaaagcagatgacaatcagtataattattatttacacagtaatgttactgaaatacaagtcttagataaagagcattatcaacaggattttgatgtgaatggttatctatatactattactatactaaattcacatctggtaatggaagttatcaatatatctgatatttatataccagtgtcacataagatagaagaagtaacatatagttgtgatggagccgttgaatgcttagcagatctcaaaaagaagaccacaaatgataatacatggtatcttaaaaaggttcatgatggcttcagctgtttaacaactactgcaactgtctgtggtgcatgtacatcaagattacattatattggcaccaaagctactacagctaaagtatctccttatattgatatccaaattaagcatggtaataagactgaagttattgaaataagagagttctcaaagtatattcatctcccatattatgttaaacctttagctcctatcttagtagaatctaatgaaatgcttatttctgaccacaaagtgttttatggacaattctgtgatcaaccaagctatggttgttttggcccgaactataaaaaagatggacaaatgtatgtactgaattcacctatggtgaaagatcctatgacacatgatagagaagtaatattagtccattgcactgaccctggtaacactgatatgaatagcttgaagtccactgattttgtctatcagaatcaaacaataatacgaccatttgaatttggaatgatttctatgggcataccaaacattggtaaactaataggtgacttctgtgaaaaacctgctgatgtcatatcagtgaatgtcaatggttgttatgattgtcaactcggtatagagatagtggtaaagtttaatataaatactaagtgtgcacaaatcaaatgtgatgttggaaaagtaagttatgaatatttcgtagactctagtagtaatacacttacattgcactctttttatgatagggaagaagttttcatcaagtgtaataagtatagtggcacattcagattagagcatagtaaggacactgactattacaagacaagtaatgaagtgcatggcagtgctacatatgattttaacctattgaaacatattcctaatttacttaccaacttcaaaacattgatccttagcataattttggtaataatggtggtatatatgttaattagtattagtaaacataccatcaagcatttcctattattgaaaaagaagagaaaattatcatataacaaaaagaatgatataagtgaagaagaagtgattgagtctattataataggcgatccagatataatggctagcatagagaagaatttcgtcatcactgatcctaggcttcccgataatcccattatctttgcatcagacggctttcttgaattgacagagtattcgcgcgaggaaatattggggagaaatgcccggtttcttcaggggccagagacagatcaagcgactgtccagaagataagagacgcaattagagatcagagggagactactgtgcagttgataaactacactaaaagcggaaagaaattctggaacttactccacctgcaacctgtgcgtgatcagaagggagagcttcaatacttcatcggtgtgcagctcgatggaagtgatcatgtatagtcaaaaatcaagtttggctctaccctttctttccaaagcttttttatacctttgtggaatggtccacccttgttcagcacgctgcagagttataatgcaacaacctgcaatggtgatttgtaacattacctcaaattaccaattgtgttgcttcatttcataatggagtaacatctgctgaactgagatatattctcatcaattaattgttttcaagcttttgtcaatttctgcgtttatatgaggagaacactact

SEQ ID NO: 21 - DNA, 44 bp, Artificial Sequence
  Synthetic Construct
ggggacaagtttgtacaaaaaagcaggcttaagtagtgaactcc

SEQ ID NO: 22 - DNA, 43 bp, Artificial Sequence
  Synthetic Construct
ggggaccactttgtacaagaaagctgggttagtagtgttctcc

SEQ ID NO: 23 - DNA, 48 bp, Artificial Sequence
  Synthetic Construct
tttcatttggagaggagtagtgaactcccacaataatttcgattaaca

SEQ ID NO: 24 - DNA, 50 bp, Artificial Sequence
  Synthetic Construct
atgccatgccgacccagtagtgttctcccacaaaaatatcaaattcaatg

SEQ ID NO: 25 - DNA, 43 bp, Artificial Sequence
  Synthetic Construct
tttcatttggagaggagtagtgaactccctataaaaccaagcg

SEQ ID NO: 26 - DNA, 44 bp, Artificial Sequence
  Synthetic Construct
atgccatgccgacccagtagtgttctccctataaacttcagcag

SEQ ID NO: 27 - DNA, 54 bp, Artificial Sequence
  Synthetic Construct
ttcatttggagaggagtagtgaactcccacaattaaatagattttgaaaaaaag

SEQ ID NO: 28 - DNA, 49 bp, Artificial Sequence
  Synthetic Construct
atgccatgccgacccagtagtgttctcccacaaattaatcaaaaaactg

SEQ ID NO: 29 - DNA, 43 bp, Artificial Sequence
  Synthetic Construct
aagcttgcaccagtgatggctagcatagagaagaatttcgtca

SEQ ID NO: 30 - DNA, 42 bp, Artificial Sequence
  Synthetic Construct
aaatatattgtaactctatacatgatcacttccatcgagctg

SEQ ID NO: 31 - DNA, 41 bp, Artificial Sequence
  Synthetic Construct
agttacaatatatttgttccaatgatggcaatatatttcat

SEQ ID NO: 32 - DNA, 26 bp, Artificial Sequence
  Synthetic Construct
cactggtgcaagctttaaaagagttt

SEQ ID NO: 33 - DNA, 41 bp, Artificial Sequence
  Synthetic Construct
atcgtctagactgtgataaatgtccatatatggtttgacag

SEQ ID NO: 34 - DNA, 43 bp, Artificial Sequence
  Synthetic Construct
atcgtctagagggttttgacaacatgatacttcaatatttttg

SEQ ID NO: 35 - DNA, 34 bp, Artificial Sequence
  Synthetic Construct
atcgtctagaatttcttcaggagaaacacggttc

SEQ ID NO: 36 - DNA, 38 bp, Artificial Sequence
  Synthetic Construct
atcgtctagaccaactagtttgtccaatttcttgaaat

SEQ ID NO: 37 - DNA, 30 bp, Artificial Sequence
  Synthetic Construct
atcgaggcctatggtttctaagggtgagga

SEQ ID NO: 38 - DNA, 26 bp, Artificial Sequence
  Synthetic Construct
cgatcccgggtcacttgtaaagctca

SEQ ID NO: 39 - DNA, 31 bp, Artificial Sequence
  Synthetic Construct
atcgaggcctcagcttctgaactttgatctg

SEQ ID NO: 40 - DNA, 26 bp, Artificial Sequence
  Synthetic Construct
cgatcccgggtcacttgtaaagctca

SEQ ID NO: 41 - DNA, 43 bp, Artificial Sequence
  Synthetic Construct
gattacgccaagcttagttggcatttgatgtaagactcaggac

SEQ ID NO: 42 - DNA, 42 bp, Artificial Sequence
  Synthetic Construct
gattacgccaagcttttgtcactgaaagaatcaacccacaga

SEQ ID NO: 43 - DNA, 43 bp, Artificial Sequence
  Synthetic Construct
gattacgccaagcttcatccattattgtgggccagtgtttacc

SEQ ID NO: 44 - DNA, 42 bp, Artificial Sequence
  Synthetic Construct
gattacgccaagcttcctgtttctatgaagccagatggtgag

SEQ ID NO: 45 - DNA, 42 bp, Artificial Sequence
  Synthetic Construct
gattacgccaagcttatattagtccattgcactgaccctggt

SEQ ID NO: 46 - DNA, 44 bp, Artificial Sequence
  Synthetic Construct
gattacgccaagcttgtggcacattcagattagagcatagtaag

SEQ ID NO: 47 - DNA, 40 bp, Artificial Sequence
  Synthetic Construct
gattacgccaagcttatctgctaagcattcaacggctcca

SEQ ID NO: 48 - DNA, 43 bp, Artificial Sequence
  Synthetic Construct
gattacgccaagctttggagttggacaatctagatgggagaac

SEQ ID NO: 49 - DNA, 41 bp, Artificial Sequence
  Synthetic Construct
gattacgccaagcttatctgctaagcattcaacggctccat

SEQ ID NO: 50 - DNA, 43 bp, Artificial Sequence; identical to SEQ ID NO: 48
  Synthetic Construct

SEQ ID NO: 51 - DNA, 43 bp, Artificial Sequence
  Synthetic Construct
gattacgccaagctttgagctctatccagctgaagtgttggct

SEQ ID NO: 52 - DNA, 43 bp, Artificial Sequence
  Synthetic Construct
gattacgccaagcttgctagagactactcctttcgatgatgct

SEQ ID NO: 53 - DNA, 41 bp, Artificial Sequence
  Synthetic Construct
gattacgccaagcttgagtccattcccatttgtcctgccag

SEQ ID NO: 54 - DNA, 44 bp, Artificial Sequence
  Synthetic Construct
gattacgccaagctttccagggacctagaaagataagaaacagc

SEQ ID NO: 55 - DNA, 43 bp, Artificial Sequence
  Synthetic Construct
gattacgccaagcttgtcttaacaaggttagggccaagctcaa

SEQ ID NO: 56 - DNA, 43 bp, Artificial Sequence
  Synthetic Construct
gattacgccaagcttccctcaagagatggactacggctacatt

SEQ ID NO: 57 - DNA, 43 bp, Artificial Sequence
  Synthetic Construct
gattacgccaagcttgaatccagctgtagattgagtttcaccc

SEQ ID NO: 58 - DNA, 42 bp, Artificial Sequence
  Synthetic Construct
gattacgccaagcttcaagattaagcttcccctgagccaaca